(19) United States
(12) Patent Application Publication
(10) Pub. No.: US 2024/0239886 A1
(43) Pub. Date: Jul. 18, 2024

(54) ANTIBODIES FOR TREATING CANCER

(71) Applicant: Yeda Research and Development Co. Ltd., Rehovot (IL)

(72) Inventors: Ido AMIT, Rehovot (IL); Chamutal BORNSTEIN-OVITS, Rehovot (IL); Adam YALIN, Rehovot (IL); Adi MOSHE, Rehovot (IL); Oren BARBOY, Rehovot (IL); Assaf WEINER, Rehovot (IL); Yonatan KATZENELENBOGEN, Rehovot (IL); Diego JAITIN, Rehovot (IL); Fadi SHEBAN, Rehovot (IL)

(73) Assignee: Yeda Research and Development Co. Ltd., Rehovot (IL)

(21) Appl. No.: 18/432,214

(22) Filed: Feb. 5, 2024

Related U.S. Application Data
(63) Continuation of application No. PCT/IL2022/050849, filed on Aug. 4, 2022.

(30) Foreign Application Priority Data
Aug. 5, 2021 (IL) 285416

(51) Int. Cl. C07K 16/28; A61K 35/17; A61K 39/00; A61K 45/06; A61K 47/64; A61P 35/00; A61P 37/04

(52) U.S. Cl. CPC C07K 16/2803; A61K 35/17; A61K 39/4611; A61K 39/4631; A61K 39/464411; A61K 45/06; A61K 47/642; A61P 35/00; A61P 37/04; A61K 2039/505; C07K 2317/565; C07K 2317/71

(57) ABSTRACT

Provided are antibodies to TREM2 and Gpnmb. Also provided are compositions comprising the antibodies and methods of using same.

### RELATED APPLICATIONS

This application is a Continuation of PCT Patent Application No. PCT/IL2022/050849 having International filing date of Aug. 4, 2022, which claims the benefit of priority of Israeli Patent Application No. 285416 filed on Aug. 5, 2021. The contents of the above applications are all incorporated by reference as if fully set forth herein in their entirety.

### SEQUENCE LISTING STATEMENT

The XML file entitled 98938SequenceListing.xml, created on Jan. 4, 2024, comprising 649,124 bytes, submitted concurrently with the filing of this application is incorporated herein by reference.

### FIELD AND BACKGROUND OF THE INVENTION

The present invention, in some embodiments thereof, relates to a method of treating cancer by reducing the immune suppressor activity of myeloid cells and, more particularly, but not exclusively, to solid cancers.

Many essential determinants of immune function cannot be precisely characterized by traditional surface markers, and it is unclear how the internal processing and integration of these signals translate toward immune activation, suppression and inflammation. Myeloid derived suppressor cells (MDSCs) are known to promote a suppressive environment for effector T cells within the tumor microenvironment (TME) and support tumor growth and immune dysfunction. Despite MDSC critical impact on treatment outcome in a broad spectrum of human disease and cancer types, their precise functional roles and molecular identity have been elusive and ill defined. MDSC do not conform to conventional surface-marker based classification schemes, and are classified using broad myeloid surface markers, various cellular assays and metabolic properties, including expression of an immune suppressive metabolic pathway expressing arginase 1 (Arg1). A thorough molecular understanding of this important and heterogeneous group of myeloid cells, based on their suppressive metabolic potential, may lead to identification of their molecular markers, pathways and activity—ultimately leading to more effective biomarkers and targeted immunotherapy.

Background art includes:
Kim et al., Cancers (Basel). 2019 Sep.; 11(9): 1315;
WO 2017/058866;
US Application No. 20180043014; and
Katzenelenbogen et al., Aug. 20;182(4):872-885.e19. doi: 10.1016/j.cell.2020.06.032. Epub 2020 Aug. 11.

### SUMMARY OF THE INVENTION

According to an aspect of some embodiments of the present invention there is provided an antibody or a fragment thereof comprising an antigen recognition domain capable of binding Triggering Receptor Expressed On Myeloid Cells 2 (TREM2), wherein the antigen recognition domain comprises the complementarity determining regions (CDRs) CDRH1, CDRH2, CDRH3, CDRL1, CDRL2 and CDRL3 or the heavy chain and light chain of an antibody selected from the group consisting of:

23A10A10
32F9E8
38C11H11
49A12D7
58B2A7
60A4F5
60H4A3
61B11C9
80E3H11
83E10B12
54H2C1
54H2C1B
23A10B10
23A10B11
38C11C10
60A4E10
60H4G2
80E3C7

According to some embodiments of the invention, the TREM2 is human TREM2.

According to some embodiments of the invention, the antigen recognition domain comprises the complementarity determining regions (CDRs) CDRH1, CDRH2, CDRH3, CDRL1, CDRL2 and CDRL3 or the heavy chain and light chain of the antibody 54H2C.

According to some embodiments of the invention, the antigen recognition domain comprises the complementarity determining regions (CDRs) CDRH1, CDRH2, CDRH3, CDRL1, CDRL2 and CDRL3 or the heavy chain and light chain of the antibody 80E3C7.

According to some embodiments of the invention, the antibody or fragment thereof is capable of inhibiting TREM2 in bone marrow derived macrophages to result in activated macrophages in vitro.

According to an aspect of some embodiments of the present invention there is provided an antibody or a fragment thereof comprising an antigen recognition domain capable of binding Transmembrane glycoprotein NMB (Gpnmb), wherein the antigen recognition domain comprises the complementarity determining regions (CDRs) CDRH1, CDRH2, CDRH3, CDRL1, CDRL2 and CDRL3 or the heavy chain and light chain of an antibody selected from the group consisting of:

g1-g2
g2-b6
g3-g2
g4-b4
g5-g2
g8-g2
g9-b4
b1-b2
b8-b8
b10-b9
b11-g2
b12-y8
b13-b7
b15-b7
b17-b17
b18-b19
b2-b2
b20-b21
b21-y8
b22-b23
b24-b26
b25-b26
y3-y22
y4-y3
y5-y5
y9-y6
y12-b4
y20-y19
y23-y20
y25-y21
y27-y22

According to some embodiments of the invention, the Gpnmb is human Gpnmb.

According to some embodiments of the invention, the antibody or fragment thereof is capable of activating CD4 T cells.

According to some embodiments of the invention, the Gpnmb is human Gpnmb.

According to some embodiments of the invention, is capable of activating CD4 T cells. According to an aspect of some embodiments of the present invention there is provided a bispecific antibody comprising in at least one arm thereof the antigen recognition domain as described herein.

According to some embodiments of the invention, the antobody or fragment thereof comprises in one arm the antibody of TREM2 and in another arm the antibody Gpnmb.

According to some embodiments of the invention, has a null or no effector function.

According to some embodiments of the invention, is IgG1.

According to some embodiments of the invention, is formulated as an antibody drug conjugate (ADC).

According to some embodiments of the invention, is formulated with a pro-inflammatory cytokine.

According to some embodiments of the invention, is conjugated to the pro-inflammatory cytokine to form a conjugate.

According to some embodiments of the invention, the pro-inflammatory cytokine is selected from the group consisting of IL-2, IL-12, IL-15, IL-21 and GM-CSF.

According to some embodiments of the invention, the conjugate comprises IL-2.

According to some embodiments of the invention, the conjugate is as set forth in SEQ ID NO: 496 and 498 or 497 and 499.

According to some embodiments of the invention, forms a chimeric antigen receptor (CAR).

According to some embodiments of the invention, as set forth in SEQ ID NO: 500.

According to an aspect of some embodiments of the present invention there is provided a cell expressing the fragment of the antibody as described herein.

According to an aspect of some embodiments of the present invention there is provided an article of manufacture comprising the antibody or antibody fragment as described herein.

According to an aspect of some embodiments of the present invention there is provided a pharmaceutical composition comprising the antibody or antibody fragment or bispecific antibody or cell as described herein and a pharmaceutically acceptable carrier or diluent.

According to an aspect of some embodiments of the present invention there is provided a method of reducing the immune suppressor activity of myeloid cells, the method comprising contacting myeloid cells with an effective amount of the antibody or antibody fragment or bispecific antibody or cell as described herein, thereby reducing the immune suppressor activity of myeloid cells.

According to an aspect of some embodiments of the present invention there is provided a method of activating CD4 T cells, the method comprising contacting CD4 T cells with an effective amount of the antibody or fragment thereof of claim 8, thereby activating the CD4 T cells.

According to an aspect of some embodiments of the present invention there is provided a method of killing myeloid cells expressing TREM2, the method comprising contacting a population of cells comprising contacting TREM2 expressing myeloid cells with an effective amount of cells as described herein, thereby killing the myeloid cells expressing TREM2.

According to some embodiments of the invention, the contacting is effected in vivo.

According to some embodiments of the invention, the contacting is effected ex vivo.

According to an aspect of some embodiments of the present invention there is provided a method of treating cancer in a subject in need thereof, the method comprising administering to the subject an effective amount of the antibody, antibody fragment, combination thereof or bispecific antibody or cell as described herein, thereby treating the cancer.

According to an aspect of some embodiments of the present invention there is provided a method of treating cancer in a subject in need thereof, the method comprising:
(a) reducing the immune suppressor activity of myeloid cells according to the method as described herein, wherein the myeloid cells are derived from the subject; and subsequently
(b) transplanting the myeloid cells to the subject, thereby treating the cancer.

According to an aspect of some embodiments of the present invention there is provided the antibody, fragment thereof, bispecific antibody or cell as described herein, for use in treating cancer.

According to some embodiments of the invention, the cancer is a solid cancer.

According to an aspect of some embodiments of the present invention there is provided the solid cancer is selected from the group consisting of lung cancer, liver cancer, ovarian cancer, gastric cancer and breast cancer.

According to some embodiments of the invention, the lung cancer is non-small cell lung cancer.

According to some embodiments of the invention, the lung cancer is small cell lung cancer. According to some embodiments of the invention, the liver cancer is Hepatocellular carcinoma.

According to some embodiments of the invention, the method or use further comprises a therapeutically effective amount of a checkpoint inhibitor.

According to some embodiments of the invention, further comprises a therapeutically effective amount of a Brutons tyrosine kinase (Btk) inhibitor.

According to some embodiments of the invention, the Brutons tyrosine kinase (Btk) inhibitor is selected from the group consisting of ibrutinib, acalabrutinib and Spebrutinib.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows SDS-PAGE analyses of hybridoma derived monoclonal antibodies against human TREM2. Secondary antibody: Peroxidase-AffiniPure Goat anti Mouse IgG, Fcg fragment specific (min X Hu, Bov, Hrs, Sr Prot).

FIG. 2 shows OD values of HEK293 sup ELISA in a binding sensitivity test of 7 anti hTREM2 antibodies.

FIG. 3 shows flow cytometer analysis of WT 293HEK cells (WT) and hTREM2 expressing HEK293 cells (hTREM2) stained with biotin conjugated anti hTREM2 antibodies following by APC-streptavidin incubation.

FIGS. 4A-B show identification of lead antibodies. Mouse bone marrow cells of TREM2 knockout (KO) and hTREM2 transgenic (hTREM2) mice were cultured 7 days in the presence of 30 ng/mL hM-CSF cytokine (Peprotech, 300-25) to generate bone marrow derived macrophage cells (BMDM). BMDM were stained with biotin conjugated anti hTREM2 leader antibodies (83E10B12, 54H2C1, 80E3C7 or IgG control) followed by APC-streptavidin incubation.

A. Representative histograms are showing staining of 83E10B12, 54H2C1, 80E3C7 versus IgG control.

B. Flow cytometry intensity of IgG control, 83E10B12, 54H2C1 and 80E3C7 of TREM2 KO or hTREM2 BMDM.

FIG. 5 shows an SPR analysis of 83E10B12, 54H2C1, 80E3C7 anti hTREM2 protein.

FIGS. 6A-B show Western blot analyses for WT and hTREM2 over-expressing (OE) TREM2 293HEK cells (A), and hTREM2 or KO BMDM (B) of 83E10B12, 54H2C1, 80E3C7 anti hTREM2 antibodies. Secondary antibody: Peroxidase-AffiniPure Goat Anti-Mouse IgG, Fcγ Fragment Specific (min X Hu,Bov,Hrs Sr Prot), (Jackson ImmunoResearch; 115-035-071)

FIG. 7 shows an immunohistochemistry analysis of TREM2 KO and hTREM2 BMDM with 83E10B12, 54H2C1, 80E3C7 anti hTREM2 antibodies.

Cells were fixed with cold methanol, washed with PBS and stained with anti hTREM2 antibody. Secondary antibody: Alexa Fluor 647-AffiniPure F(ab′)2 Fragment Donkey Anti-Mouse IgG (H+L) (Jackson ImmunoResearch; 715-606).

FIGS. 8A-C show that mAb 54H2C1 and 80E3C7 block hTREM2 activity in BMDM culture.

(A) Single cell map of BMDM culture for TREM2KO and hTREM2 bone marrow cells. (B) density plots highlighting cells from hTREM2 (cyan) or TREM2-KO (red) mice on the single cell map at days 2,3,4,5,6,7 during BMDM differentiation. (c)

Quantification of TREM2+GPNMB+ and TREM2- macrophages at day 7 of BMDM cultures from WT or TREM2-KO cells and for WT cells treated with mAb 54H2C1, 80E3C7 or IgG control at day 2.

FIG. 9 shows an ELISA analysis of biotin conjugated hTREM2 antibody penetration in humanized TREM2 mice harboring MCA-205 induced tumor.

FIGS. 10A-B show that hGPNMB protein suppresses CD4 T cell activation. CFSE stained human CD4 T cells were incubated in pre-coated anti CD3, CD2 and CD28 antibodies for activation and proliferation for 3 days. Number of replication was calculated by CFSE intensity measurement by flow cytometry, IFNg secretion measurement done by ELISA (Biolegend, BLG-430104).

FIG. 11 is a curve of mean fluorescence intensity (MFI) E3C7 antibody (yellow) and IgG control (gray) staining human M2 macrophage, at indicated antibody concentrations. Biotinylated antibodies were used, following by PE-streptavidin binding.

FIGS. 12A-B show the effect if E3C7 on macrophage polarization. Human CD14+ monocytes were purified from peripheral blood of 3 healthy donors and differentiated into macrophages by hM-CSF administration for five days, followed by polarization to “M2” macrophage by IL-4 administration. E3C7 Ab anti-TREM2 antibody was added to culture on days 3 and 5 of the assay. (A) qPCR and (B) ELISA were performed 24 h after the treatment with IL4.

FIGS. 13A-C show that mAb E3C7 blocks hTREM2 activity in human macrophage differentiation in culture. (A) Single cell map of human in vitro differentiated macrophage with or without the presence of IL-4 cytokine and with or without anti TREM2/IgG control antibody. (B) Density plots highlighting cells from M-CSF (pink) or M-CSF+IL-4 (blue) samples on the single cell map. (C) Density plots highlighting cells from anti TREM2 (pink), IgG control (green), or no antibody (blue) samples on the single cell map.

FIG. 14 shows that mAb E3C7 blocks hTREM2 activity in human macrophage differentiation in culture. Dot plots visualization shows expression of differentially expressed genes in two differentiation conditions: M-SCF differentiated macrophages, M-CSF+IL4 differentiated macrophages, reated with either anti-TREM2 mAb, IgG control or no antibody treatment.

FIGS. 15A-B shows that antibody E3C7 remodels mouse tumor microenvironment (TME). (A) Percentage of TAM with high Type-I interferon signaling in E3C7 treated mice compared to IgG control. (B) Percentage of dysfunctional CD8 T-cells (PD1+LAG3+) in E3C7 treated mice compared to IgG control.

FIG. 16 is a Volcano plot showing differential gene expression in tumor associated macrophages in E3C7 treated mice. The plot shows differentially expressed genes in TME macrophages treated with E3C7 vs. IgG control.

FIGS. 17A-B show that anti TREM2 antibody E3C7 and inflammatory cytokines (IL2, IL-15) synergize to elevate pro-inflammatory phenotype of macrophage. (A) Gene expression analysis following treatment of anti TREM2, GM-CSF, IL12, IL15, IL2 or a combination of anti TREM2 with one of the cytokines. (B) M2 macrophage cytokines secretion analysis following anti TREM2 and IL-2/IL-15 cytokine treatments.

FIGS. 18A-B show that the anti TREM2 antibody E3C7 and inflammatory cytokines (IL2, IL-15) synergize to elevate pro-inflammatory phenotype of M2 macrophage and consequently release the CD8 T cells activation suppression by M2 macrophage. (A) the cell trace dye CFSE stained human CD8 T cells were co-culture with M2 macrophage, treated with anti TREM2 antibody and IL2/IL-15 cytokine, in pre-coated anti CD3 and CD28 antibodies for activation and proliferation for 3 days. Percentage of proliferative cells calculated by CFSE intensity measurement by flow cytometry. (B) CFSE stained human CD8 T cells were co-culture with M2 macrophage in pre-coated anti CD3 and CD28 antibodies, in the presence of IL-2/IL-15 cytokine, for activation and proliferation for 3 days. Percentage of proliferative cells calculated by CFSE intensity measurement by flow cytometry.

FIGS. 19A-D are graphs showing binding of anti-hTREM2-cytokine fusions. Plates were coated with recombinant hTREM2 (2 μg/ml) and were used for direct ELISA assay with the recombinant antibodies at different concentrations. Alternatively, hM2 cells supernatant was used for soluble TREM2 protein binding assay (Sandwich Elisa). Different secondary antibodies were used: A,C. anti-human IgG; B,D; Anti-hIL2 (BLG-500302).

FIGS. 20A-B show that anti-hTrem-IL2 Ab according to an embodiment of the invention and fusions thereof with IL-2 activate hCD8+/CD4+ cell in culture. hCD8/CD4+ cells were cultured with or without activation with the antibodies (10 μg/ml) or with recombinant proteins (100 ng/ml). A. Percentage of proliferating CD8+ cell at 4 days. B. Secretion of IFNg at 24 hrs and 4 days (μg/ml).

FIG. 21 shows IFNg release under different co-culture conditions, as determined by IFNg concentration in the medium. Statistical significance testing was done using Holm-Šídák's multiple comparisons test (16 replicates in each condition).

FIGS. 22A-F are graphs showing the activity of TREM2 CAR-T cells. (A) IFN-γ ELISA assay of TREM2-CAR T cells co-cultured with TREM2+HEK293. (B) Flow cytometry analysis for activation and killing of TREM2+HEK293 by TREM2-CAR T cells co-cultured therewith. (C) Flow cytometry FSC-SSC gating of human TAM-like cells after 24h co-culture with TREM2-CAR T cells and Mock CAR T cells. (D) IFN-γ ELISA assay of TREM2-CAR T cells co-cultured with human TAM-like cells (E) Flow cytometry analysis for activation and killing of TREM2-CAR T cells of TREM2-CAR T cells co-cultured with human TAM-like cells. (F) IFN-γ ELISA assay of TREM2-CAR T cells co-cultured with BMDM and BMDC from humanized TREM2 mice, TREM2ko mice and wt mice.

FIG. 23 is a schematic illustration of a TREM2 chimeric antigen receptor according to some embodiments of the invention.

FIG. 24 shows a schametic illustration and sequences of anti TREM2-IL2 fusions of some embodiments of the invention.

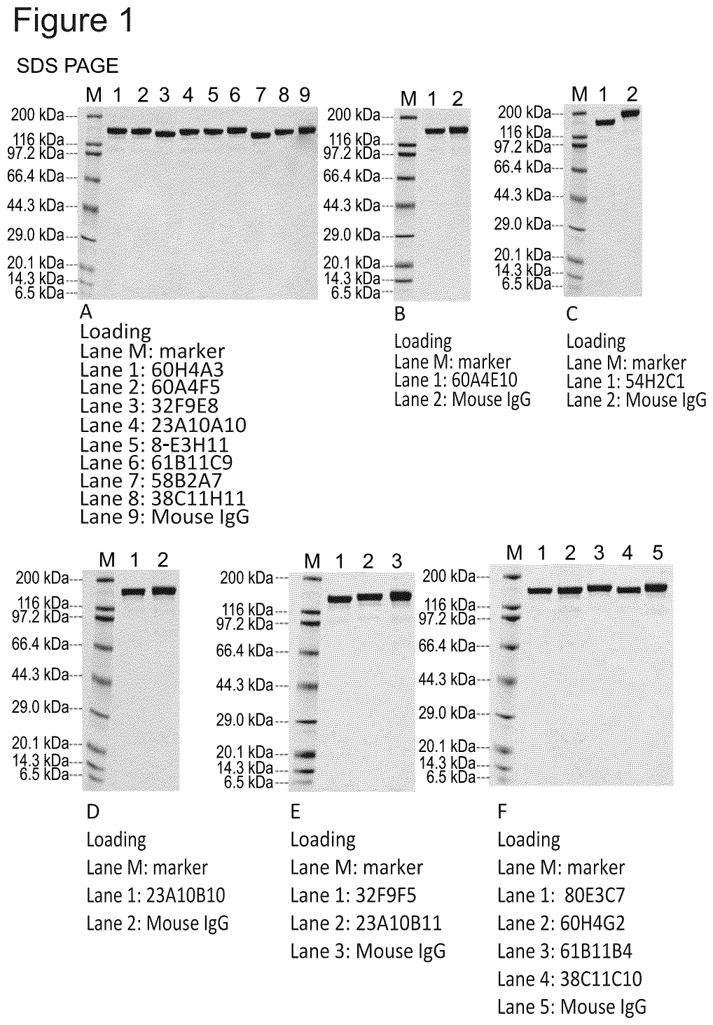

### DESCRIPTION OF SPECIFIC EMBODIMENTS OF THE INVENTION

The present invention, in some embodiments thereof, relates to a method of treating cancer by reducing the immune suppressor activity of myeloid cells and, more particularly, but not exclusively, to solid cancers.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors analyzed suppressive metabolic circuits within the tumor microenvironment using the direct targeting of Arg1⁺ myeloid cells. They identified two distinct populations of Arg1⁺ TREM2⁺ cells in the tumor, a tumor associated macrophage population and a unique population of Mreg, characterized by defined surface markers (e.g. Gpnmb), and signaling, including hypoxia. They demonstrated the suppressive activity of the Arg1+TAM and Mreg populations over CD8 T cells. The present findings identified TREM2 as a marker and potential regulator of suppressive myeloid cells. Genetic ablation of TREM2 in mice, led to dramatic decrease in the Mreg population with increase in immune reactivity towards the tumor, including decrease in dysfunctional CD8+ T cells and increase in NK and cytotoxic T cells. The results suggest that specific targeting of the Mreg population will be more beneficial than targeting the tumor associated macrophage population for the treatment of cancer.

Hence the present inventors have previously suggested a regulating regulatory myeloid cell population (Mreg) by co-targeting Triggering Receptor Expressed On Myeloid Cells 2 (TREM2) and Transmembrane glycoprotein NMB (Gpnmb) for the treatment of cancer. The present inventors have now identified antibodies for TREM2 and Gpnmb that can be used in such co-targeting.

Anti TREM2 antibodies were screened by employing a unique screening assay whereby bone marrow derived macrophages are activated to acquire an M1 profile in the presence of the screened antibodies. This activation is a direct result of TREM2 blocking (loss-of-function) and it mimics a TREM2 knock out phenotype as disclosed in the Examples section which follows. Whereas, anti Gpnmb binders are selected based on their ability to inhibit Mreg suppressing CD4 T cell activation, and by that activating CD4 T cells.

The ability to activate CD4 T cells and macrophages renders the present antibodies beneficial for usein the clinic and especially in the treatment of cancer.

Functional characterization of anti TREM2 antibodies of some embodiments of the invention showed high affinity recognition (low nanomolar to picomolar range), inhibition of TREM2 function in myeloid cells as evidenced by their ability to reprogram immunosuppressive macrophages toward pro-inflammatory monocyte-like cells. Comparative single-cell RNAseq analysis revealed near-identical transcriptome patterns of antibody-treated, reprogrammed BMDM and those derived from TREM2^{-/-} mice. Anti-TREM2 antibodies of some embodiments of the invention attenuated tumor growth and reprograms tumor macrophages in humanized mice bearing tumors, as evidenced by differential increase in Type-I IFN genes. A combination of anti-TREM2 antibody with pro-inflammatory cytokines or conjugation of same showed synergic enhancement in myeloid reprogramming and T-cell activation. In addition, TREM2-CAR T cells generated using antibodies of some embodiments of the invention are able to initiate an effective cytotoxic response and deplete human TREM2 expressing myeloid cells and immunosuppressive macrophages. The observed effect of TREM2-CAR T cells is an important milestone towards development of myeloid-centric immunotherapy of cancer.

Thus, according to an aspect of the invention, there is provided an antibody or a fragment thereof comprising an antigen recognition domain capable of binding Triggering Receptor Expressed On Myeloid Cells 2 (TREM2), wherein said antigen recognition domain comprises the complementarity determining regions (CDRs) CDRH1, CDRH2, CDRH3, CDRL1, CDRL2 and CDRL3 or the heavy chain and light chain of an antibody selected from the group consisting of:

23A10A10
32F9E8
38C11H11
49A12D7
58B2A7
60A4F5
60H4A3
61B11C9
80E3H11
83E10B12
54H2C1
54H2C1B
23A10B10
23A10B11
38C11C10
60A4E10
60H4G2
80E3C7

TREM2 is an immunoglobulin-like receptor primarily expressed on myeloid lineage cells, including without limitation, macrophages, dendritic cells, osteoclasts, microglia, monocytes, Langerhans cells of skin, and Kupffer cells. In some embodiments, TREM2 forms a receptor-signaling complex with DAP12. In some embodiments, TREM2 phosphorylates and signals through DAP12 (an ITAM domain adaptor protein). In some embodiments TREM2 signaling results in the downstream activation of PI3K. In some embodiments TREM2 signaling results in the downstream phosphorylation of spleen tyrosine kinase (stk).

TREM2 proteins of the present disclosure include, without limitation, a mammalian TREM2 protein including but not limited to human TREM2 protein (Uniprot Accession No. Q9NZC2), mouse TREM2 protein (Uniprot Accession No. Q99NH8), rat TREM2 protein (Uniprot Accession No. D3ZZ89), Rhesus monkey TREM2 protein (Uniprot Accession No. F6QVF2), bovine TREM2 protein (Uniprot Accession No. Q05B59), equine TREM2 protein (Uniprot Accession No. F7D6L0), pig TREM2 protein (Uniprot Accession No. H2EZZ3), and dog TREM2 protein (Uniprot Accession No. E2RP46).

An exemplary human TREM2 amino acid sequence is set forth below as SEQ ID NO: 1.

In some embodiments, the human TREM2 is a preprotein that includes a signal peptide. In some embodiments, the human TREM2 is a mature protein. In some embodiments, the mature TREM2 protein does not include a signal peptide. In some embodiments, the mature TREM2 protein is expressed on a cell. In some embodiments, TREM2 contains a signal peptide located at amino acid residues 1-18 of human TREM2 (SEQ ID NO: 1); an extracellular immunoglobulin-like variable-type (IgV) domain located at amino acid residues 29-112 of human TREM2 (SEQ ID NO: 1); additional extracellular sequences located at amino acid residues 113-174 of human TREM2 (SEQ ID NO: 1); a transmembrane domain located at amino acid residues 175-195 of human TREM2 (SEQ ID NO: 1); and an intracellular domain located at amino acid residues 196-230 of human TREM2 (SEQ ID NO: 1). According to a specific embodiment the TREM2 is human TREM2.

According to a specific embodiment, the antigen recognition domain comprises the complementarity determining regions (CDRs) CDRH1, CDRH2, CDRH3, CDRL1, CDRL2 and CDRL3 or the heavy chain and light chain of the antibody 54H2C.

According to a specific embodiment, the antigen recognition domain comprises the complementarity determining regions (CDRs) CDRH1, CDRH2, CDRH3, CDRL1, CDRL2 and CDRL3 or the heavy chain and light chain of the antibody 80E3C7 (also referred to as “E3C7”).

According to a specific embodiment, the antibody or antibody fragment is capable of inhibiting TREM2 in bone marrow derived macrophages to result in activated macrophages in vitro, which is typical of TREM2 knock-out.

In other terms, the antibody or antibody fragment or bispecific antibody, is an inhibitory antibody for TREM2. This can be explained by the resemblance of the phenotype following incubation, to TREM2 knock-out cells (see Examples section).

Specifically, the present inventors found that bone marrow derived macrophages (BMDM) express high amount of TREM2. The antibodies of some embodiments of the invention bind TREM2-BMDM, as well as

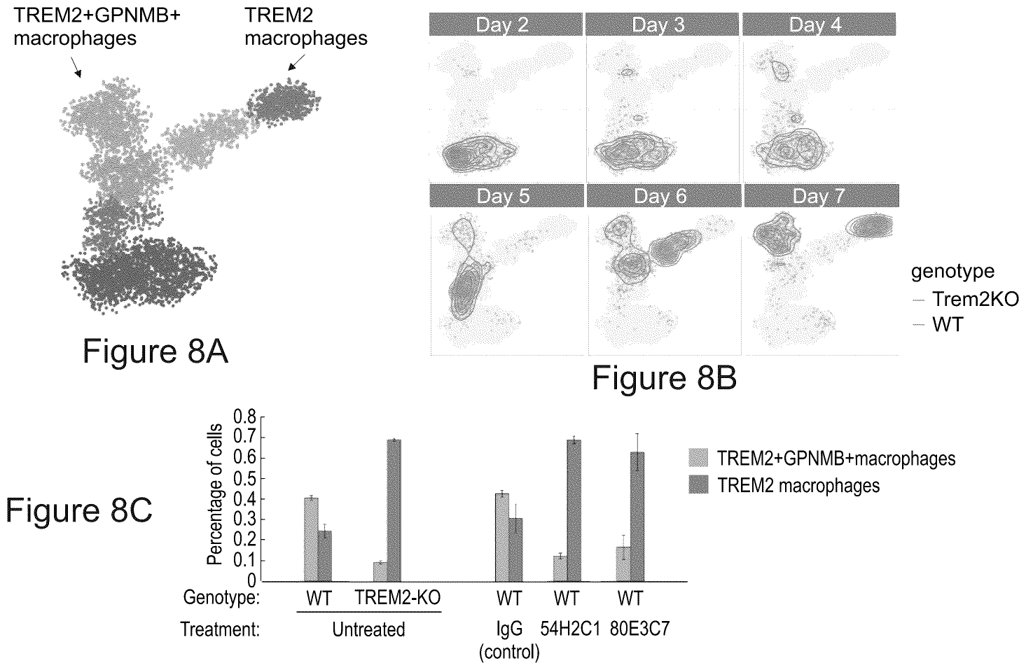

BMDM are known to produce high levels of suppressive cytokines such as IL-10 and TGF-β). The effect of the antibodies of BMDM on temporal maturation trajectory can be determined using single cell RNA-seq. An effect can be seen 2-7 days following activation. The effect is typically the acquirement of an M1 phenotype. Thus, as can be seen in the Examples section which follows, the WT hTREM2 BM cells showed an M2-phenotype at day 7 with high expression of Gpnmb, Lpl, Anxa1, Mmp12, Adam8, Lgals1, Lgals3, Spp1 and Lilrb4a while TREM2-KO BM genotype displayed an activated M1 phenotype, including Selenop, Ms4a4a, Fcgr2b, Ms4a7 and Lyz2 (FIGS. 8A-B). To screen for antibodies with antagonistic activity for TREM2 hTREM2 mouse bone marrow cells are cultured with M-CSF and anti hTREM2 antibodies or IgG isotype to the medium at day 2 and 5 of culturing. Using single cell RNA-seq the cells at day 7 are characterized and quantified for the distribution of cells between M2-phenotype (TREM2+Gpnmb+) and M1-phenotype (TREM2-) at each condition.

As can be seen in FIG. 8C, more than 70% of TREM2-KO cells reached an M1-phenotype with less than 10% showing M2 phenotype, in contrast hTREM2 cells showed 40% M2- phenotype and only 24% M1- phenotype. Adding IgG isotype mAb to the culture did not change significantly the M1/M2 ratio and showed a similar outcome as the untreated culture, while adding anti-hTREM2 mAb 54H2C1 or 80E3C7 dramatically reduced the percentage of the M2 phenotype to 12% and 16% respectively with an increase in M1 phenotype to 69% and 63% (FIG. 8C), showing very similar maturation trajectory to TREM2-KO cells.

As used herein “M1 macrophages” are macrophages that express Selenop, Ms4a4a, Fcgr2b, Ms4a7 and Lyz2.

As used herein “M2 macrophages” are macrophages that express Gpnmb, Lpl, Anxa1, Mmp12, Adam8, Lgals1, Lgals3, Spp1 and Lilrb4a.

According to an additional or an alternative aspect, there is provided an antibody or a fragment thereof comprising an antigen recognition domain capable of binding Transmembrane glycoprotein NMB (Gpnmb), wherein said antigen recognition domain comprises the complementarity determining regions (CDRs) CDRH1, CDRH2, CDRH3, CDRL1, CDRL2 and CDRL3 or the heavy chain and light chain of an antibody selected from the group consisting of:

g1-g2
g2-b6
g3-g2
g4-b4
g5-g2
g8-g2
g9-b4
b1-b2
b8-b8
b10-b9
b11-g2
b12-y8
b13-b7
b15-b7
b17-b17
b18-b19
b2-b2
b20-b21
b21-y8
b22-b23
b24-b26
b25-b26
y3-y22
y4-y3
y5-y5
y9-y6
y12-b4
y20-y19
y23-y20
y25-y21
y27-y22

Tables A and B below list the SEQ ID NOs of the sequences of each antibody. Each antibody should be considered as an individual embodiment.

TABLE A
Anti TREM2 Antibodies

| Antibody # | SEQ ID NO: | |
|---|---|---|
| | heavy Chain DNA | |
| 23A10A10 | 6 | |
| 32F9E8 | 7 | |
| 38C11H11 | 8 | |
| 49A12D7 | 9 | |
| 58B2A7 | 10 | |
| 60A4F5 | 11 | |
| 60H4A3 | 12 | |
| 61B11C9 | 13 | |
| 80E3H11 | 14 | |
| 83E10B12 | 15 | |
| 54H2C1 | 16 | |
| 54H2C1B | 17 | |
| 23A10B10 | 18 | |
| 23A10B11 | 19 | |
| 38C11C10 | 20 | |
| 60A4E10 | 21 | |
| 60H4G2 | 22 | |
| 80E3C7 | 23 | |
| | Heavy Chain protein | |
| 23A10A10 | 24 | |
| 32F9E8 | 25 | |
| 38C11H11 | 26 | |
| 49A12D7 | 27 | |
| 58B2A7 | 28 | |
| 60A4F5 | 29 | |
| 60H4A3 | 30 | |
| 61B11C9 | 31 | |
| 80E3H11 | 32 | |
| 83E10B12 | 33 | |
| 54H2C1 | 34 | |
| 54H2C1B | 35 | |
| 23A10B10 | 36 | |
| 23A10B11 | 37 | |
| 38C11C10 | 38 | |
| 60A4E10 | 39 | |
| 60H4G2 | 40 | |
| 80E3C7 | 41 | |
| | Light Chain DNA | |
| 23A10A10 | 42 | |
| 32F9E8 | 43 | |
| 38C11H11 | 44 | |
| 49A12D7 | 45 | |
| 58B2A7 | 46 | |
| 60A4F5 | 47 | |
| 60H4A3 | 48 | |
| 61B11C9 | 49 | |
| 80E3H11 | 50 | |
| 83E10B12 | 51 | |
| 54H2C1 | 52 | |
| 54H2C1B | 53 | |
| 23A10B10 | 54 | |
| 23A10B11 | 55 | |
| 38C11C10 | 56 | |
| 60A4E10 | 57 | |
| 60H4G2 | 58 | |
| 80E3C7 | 59 | |
| | Light chain protein | |
| 23A10A10 | 60 | |
| 32F9E8 | 61 | |
| 38C11H11 | 62 | |
| 49A12D7 | 63 | |
| 58B2A7 | 64 | |
| 60A4F5 | 65 | |
| 60H4A3 | 66 | |
| 61B11C9 | 67 | |
| 80E3H11 | 68 | |
| 83E10B12 | 69 | |
| 54H2C1 | 70 | |
| 54H2C1B | 71 | |
| 23A10B10 | 72 | |
| 23A10B11 | 73 | |
| 38C11C10 | 74 | |
| 60A4E10 | 75 | |
| 60H4G2 | 76 | |
| 80E3C7 | 77 | |
| | CDRs | |
| | 23A10A10 | |
| Heavy | 78 | |
| Heavy | 79 | |
| Heavy | 80 | |
| Light | 81 | |
| Light | | Ala Ala Ser |
| Light | 83 | |
| | 32F9E8 | |
| Heavy | 84 | |
| Heavy | 85 | |
| Heavy | 86 | |
| Light | 87 | |
| Light | | Leu Val Ser |
| Light | 89 | |
| | 38C11H11 | |
| Heavy | 90 | |
| Heavy | 91 | |
| Heavy | 92 | |
| Light | 93 | |
| Light | | Leu Val Ser |
| Light | 95 | |
| | 49A12D7 | |
| Heavy | 96 | |
| Heavy | 97 | |
| Heavy | 98 | |
| Light | 99 | |
| Light | | Leu Val Ser |
| Light | 101 | |
| | 58B2A7 | |
| Heavy | 102 | |
| Heavy | 103 | |
| Heavy | 104 | |
| Light | 105 | |
| Light | | Leu Val Ser |
| Light | 107 | |
| | 60A4F5 | |
| Heavy | 108 | |
| Heavy | 109 | |
| Heavy | 110 | |
| Light | 111 | |
| Light | | Lys Val Ser |
| Light | 113 | |
| | 60H4A3 | |
| Heavy | 114 | |
| Heavy | 115 | |
| Heavy | 116 | |
| Light | 117 | |
| Light | | Tyr Ala Ser |
| Light | 119 | |
| | 61B11C9 | |
| Heavy | 120 | |
| Heavy | 121 | |
| Heavy | 122 | |
| Light | 123 | |
| Light | | Trp Ala Ser |
| Light | 125 | |
| | 80E3H11 | |
| Heavy | 126 | |
| Heavy | 127 | |
| Heavy | 128 | |
| Light | 129 | |
| Light | | Leu Val Ser |
| Light | 131 | |
| | 83E10B12 | |
| Heavy | 132 | |
| Heavy | 133 | |
| Heavy | 134 | |
| Light | 135 | |
| Light | | Tyr Thr Ser |
| Light | 137 | |
| | 54H2C1 | |
| Heavy | 138 | |
| Heavy | 139 | |
| Heavy | 140 | |
| Light | 141 | |
| Light | | Lys Val Ser |
| Light | 143 | |
| | 54H2C1B | |
| Heavy | 144 | |
| Heavy | 145 | |
| Heavy | 146 | |
| Light | 147 | |
| Light | | Leu Val Ser |
| Light | 149 | |
| | 23A10B10 | |
| Heavy | 150 | |
| Heavy | 151 | |
| Heavy | 152 | |
| Light | 153 | |
| Light | | Ala Ala Ser |
| Light | 155 | |
| | 23A10B11 | |
| Heavy | 156 | |
| Heavy | 157 | |
| Heavy | 158 | |
| Light | 159 | |
| Light | | Ala Ala Ser |
| Light | 161 | |
| | 38C11C10 | |
| Heavy | 162 | |
| Heavy | 163 | |
| Heavy | 164 | |
| Light | 165 | |
| Light | | Leu Val Ser |
| Light | 167 | |
| | 60A4E10 | |
| Heavy | 168 | |
| Heavy | 169 | |
| Heavy | 170 | |
| Light | 171 | |
| Light | | Lys Val Cys |
| Light | 173 | |
| | 60H4G2 | |
| Heavy | 174 | |
| Heavy | 175 | |
| Heavy | 176 | |
| Light | 177 | |
| Light | | Trp Ala Ser |
| Light | 179 | |
| | 80E3C7 | |
| Heavy | 180 | |
| Heavy | 181 | |
| Heavy | 182 | |
| Light | 183 | |
| Light | | Lys Val Ser |
| Light | 185 | |

TABLE B
Anti Gpnmb Antibodies

| Antibody # | SEQ ID NO: | |
|---|---|---|
| | heavy Chain DNA | |
| g1-g2 | 186 | |
| g2-b6 | 187 | |
| g3-g2 | 188 | |
| g4-b4 | 189 | |
| g5-g2 | 190 | |
| g8-g2 | 191 | |
| g9-b4 | 192 | |
| b1-b2 | 193 | |
| b8-b8 | 194 | |
| b10-b9 | 195 | |
| b11-g2 | 196 | |
| b12-y8 | 197 | |
| b13-b7 | 198 | |
| b15-b7 | 199 | |
| b17-b17 | 200 | |
| b18-b19 | 201 | |
| b2-b2 | 202 | |
| b20-b21 | 203 | |
| b21-y8 | 204 | |
| b22-b23 | 205 | |
| b24-b26 | 206 | |
| b25-b26 | 207 | |
| y3-y22 | 208 | |
| y4-y3 | 209 | |
| y5-y5 | 210 | |
| y9-y6 | 211 | |
| y12-b4 | 212 | |
| y20-y19 | 213 | |
| y23-y20 | 214 | |
| y25-y21 | 215 | |
| y27-y22 | 216 | |
| | Heavy Chain protein | |
| g1-g2 | 217 | |
| g2-b6 | 218 | |
| g3-g2 | 219 | |
| g4-b4 | 220 | |
| g5-g2 | 221 | |
| g8-g2 | 222 | |
| g9-b4 | 223 | |
| b1-b2 | 224 | |
| b8-b8 | 225 | |
| b10-b9 | 226 | |
| b11-g2 | 227 | |
| b12-y8 | 228 | |
| b13-b7 | 229 | |
| b15-b7 | 230 | |
| b17-b17 | 231 | |
| b18-b19 | 232 | |
| b2-b2 | 233 | |
| b20-b21 | 234 | |
| b21-y8 | 235 | |
| b22-b23 | 236 | |
| b24-b26 | 237 | |
| b25-b26 | 238 | |
| y3-y22 | 239 | |
| y4-y3 | 240 | |
| y5-y5 | 241 | |
| y9-y6 | 242 | |
| y12-b4 | 243 | |
| y20-y19 | 244 | |
| y23-y20 | 245 | |
| y25-y21 | 246 | |
| y27-y22 | 247 | |
| | Light Chain DNA | |
| g1-g2 | 248 | |
| g2-b6 | 249 | |
| g3-g2 | 250 | |
| g4-b4 | 251 | |
| g5-g2 | 252 | |
| g8-g2 | 253 | |
| g9-b4 | 254 | |
| b1-b2 | 255 | |
| b8-b8 | 256 | |
| b10-b9 | 257 | |
| b11-g2 | 258 | |
| b12-y8 | 259 | |
| b13-b7 | 260 | |
| b15-b7 | 261 | |
| b17-b17 | 262 | |
| b18-b19 | 263 | |
| b2-b2 | 264 | |
| b20-b21 | 265 | |
| b21-y8 | 266 | |
| b22-b23 | 267 | |
| b24-b26 | 268 | |
| b25-b26 | 269 | |
| y3-y22 | 270 | |
| y4-y3 | 271 | |
| y5-y5 | 272 | |
| y9-y6 | 273 | |
| y12-b4 | 274 | |
| y20-y19 | 275 | |
| y23-y20 | 276 | |
| y25-y21 | 277 | |
| y27-y22 | 278 | |
| | Light chain protein | |
| g1-g2 | 279 | |
| g2-b6 | 280 | |
| g3-g2 | 281 | |
| g4-b4 | 282 | |
| g5-g2 | 283 | |
| g8-g2 | 284 | |
| g9-b4 | 285 | |
| b1-b2 | 286 | |
| b8-b8 | 287 | |
| b10-b9 | 288 | |
| b11-g2 | 289 | |
| b12-y8 | 290 | |
| b13-b7 | 291 | |
| b15-b7 | 292 | |
| b17-b17 | 293 | |
| b18-b19 | 294 | |
| b2-b2 | 295 | |
| b20-b21 | 296 | |
| b21-y8 | 297 | |
| b22-b23 | 298 | |
| b24-b26 | 299 | |
| b25-b26 | 300 | |
| y3-y22 | 301 | |
| y4-y3 | 302 | |
| y5-y5 | 303 | |
| y9-y6 | 304 | |
| y12-b4 | 305 | |
| y20-y19 | 306 | |
| y23-y20 | 307 | |
| y25-y21 | 308 | |
| y27-y22 | 309 | |
| | CDRs | |
| | g1-g2 | |
| Heavy | 310 | |
| Heavy | 311 | |
| Heavy | 312 | |
| Light | 313 | |
| Light | | Leu Val Ser |
| Light | 315 | |
| | g2-b6 | |
| Heavy | 316 | |
| Heavy | 317 | |
| Heavy | 318 | |
| Light | 319 | |
| Light | | Tyr Ser Ser |
| Light | 321 | |
| | g3-g2 | |
| Heavy | 322 | |
| Heavy | 323 | |
| Heavy | 324 | |
| Light | 325 | |
| Light | | Leu Val Ser |
| Light | 327 | |
| | g4-b4 | |
| Heavy | 328 | |
| Heavy | 329 | |
| Heavy | 330 | |
| Light | 331 | |
| Light | | Ser Thr Ser |
| Light | 333 | |
| | g5-g2 | |
| Heavy | 334 | |
| Heavy | 335 | |
| Heavy | 336 | |
| Light | 337 | |
| Light | | Leu Val Ser |
| Light | 339 | |
| | g8-g2 | |
| Heavy | 340 | |
| Heavy | 341 | |
| Heavy | 342 | |
| Light | 343 | |
| Light | | Leu Val Ser |
| Light | 345 | |
| | g9-b4 | |
| Heavy | 346 | |
| Heavy | 347 | |
| Heavy | 348 | |
| Light | 349 | |
| Light | | Ser Thr Ser |
| Light | 351 | |
| | b1-b2 | |
| Heavy | 352 | |
| Heavy | 353 | |
| Heavy | 354 | |
| Light | 355 | |
| Light | | Asp Thr Ser |
| Light | 357 | |
| | b8-b8 | |
| Heavy | 358 | |
| Heavy | 359 | |
| Heavy | 360 | |
| Light | 361 | |
| Light | | Arg Ala Asn |
| Light | 363 | |
| | b10-b9 | |
| Heavy | 364 | |
| Heavy | 365 | |
| Heavy | 366 | |
| Light | 367 | |
| Light | | His Thr Ser |
| Light | 369 | |
| | b11-g2 | |
| Heavy | 370 | |
| Heavy | 371 | |
| Heavy | 372 | |
| Light | 373 | |
| Light | | Leu Val Ser |
| Light | 375 | |
| | b12-y8 | |
| Heavy | 376 | |
| Heavy | 377 | |
| Heavy | 378 | |
| Light | 379 | |
| Light | | Lys Val Ser |
| Light | 381 | |
| | b13-b7 | |
| Heavy | 382 | |
| Heavy | 383 | |
| Heavy | 384 | |
| Light | 385 | |
| Light | | Arg Thr Asn |
| Light | 387 | |
| | b15-b7 | |
| Heavy | 388 | |
| Heavy | 389 | |
| Heavy | 390 | |
| Light | 391 | |
| Light | | Arg Thr Asn |
| Light | 393 | |
| | b17-b17 | |
| Heavy | 394 | |
| Heavy | 395 | |
| Heavy | 396 | |
| Light | 397 | |
| Light | | Arg Thr Ser |
| Light | 399 | |
| | b18-b19 | |
| Heavy | 400 | |
| Heavy | 401 | |
| Heavy | 402 | |
| Light | 403 | |
| Light | | Asp Thr Ser |
| Light | 405 | |
| | b2-b2 | |
| Heavy | 406 | |
| Heavy | 407 | |
| Heavy | 408 | |
| Light | 409 | |
| Light | | Asp Thr Ser |
| Light | 411 | |
| | b20-b21 | |
| Heavy | 412 | |
| Heavy | 413 | |
| Heavy | 414 | |
| Light | 415 | |
| Light | | Arg Thr Ser |
| Light | 417 | |
| | b21-y8 | |
| Heavy | 418 | |
| Heavy | 419 | |
| Heavy | 420 | |
| Light | 421 | |
| Light | | Lys Val Ser |
| Light | 423 | |
| | b22-b23 | |
| Heavy | 424 | |
| Heavy | 425 | |
| Heavy | 426 | |
| Light | 427 | |
| Light | | Lys Val Ser |
| Light | 429 | |
| | b24-b26 | |
| Heavy | 430 | |
| Heavy | 431 | |
| Heavy | 432 | |
| Light | 433 | |
| Light | | Asp Thr Ser |
| Light | 435 | |
| | b25-b26 | |
| Heavy | 436 | |
| Heavy | 437 | |
| Heavy | 438 | |
| Light | 439 | |
| Light | | Asp Thr Ser |
| Light | 441 | |
| | y3-y22 | |
| Heavy | 442 | |
| Heavy | 443 | |
| Heavy | 444 | |
| Light | 445 | |
| Light | | Ser Thr Ser |
| Light | 447 | |
| | y4-y3 | |
| Heavy | 448 | |
| Heavy | 449 | |
| Heavy | 450 | |
| Light | 451 | |
| Light | | Trp Ala Ser |
| Light | 453 | |
| | y5-y5 | |
| Heavy | 454 | |
| Heavy | 455 | |
| Heavy | 456 | |
| Light | 457 | |
| Light | | Leu Thr Ser |
| Light | 459 | |
| | y9-y6 | |
| Heavy | 460 | |
| Heavy | 461 | |
| Heavy | 462 | |
| Light | 463 | |
| Light | | Tyr Ile Ser |
| Light | 465 | |
| | y12-b4 | |
| Heavy | 466 | |
| Heavy | 467 | |
| Heavy | 468 | |
| Light | 469 | |
| Light | | Ser Thr Ser |
| Light | 471 | |
| | y20-y19 | |
| Heavy | 472 | |
| Heavy | 473 | |
| Heavy | 474 | |
| Light | 475 | |
| Light | | Tyr Thr Ser |
| Light | 477 | |
| | y23-y20 | |
| Heavy | 478 | |
| Heavy | 479 | |
| Heavy | 480 | |
| Light | 481 | |
| Light | | Ser Thr Ser |
| Light | 483 | |
| | y25-y21 | |
| Heavy | 484 | |
| Heavy | 485 | |
| Heavy | 486 | |
| Light | 487 | |
| Light | | Arg Ala Asn |
| Light | 489 | |
| | y27-y22 | |
| Heavy | 490 | |
| Heavy | 491 | |
| Heavy | 492 | |
| Light | 493 | |
| Light | | Ser Thr Ser |
| Light | 495 | |

Transmembrane glycoprotein NMB (GPNMB) is a type IA cell-surface glycoprotein that in humans is encoded by the GPNMB gene. Two transcript variants encoding 560 and 572 amino acid isoforms have been characterized for this gene in humans. The 470 aa long fragment is the extracellular domain used for mouse immunization. The mouse and rat orthologues of GPNMB are known as DC-HIL and Osteoactivin, respectively. An exemplary GPNMB has an amino acid sequence as set forth in SEQ ID NO: 2.

According to a specific embodiment, the Gpnmb is human Gpnmb (SEQ ID No: 2).

According to a specific embodiment, the antibody, fragment thereof or bispecific antibody is capable of activating CD4 T cells.

Activation of CD4+T cells occurs through the simultaneous engagement of the T-cell receptor and a co-stimulatory molecule (like CD28, or ICOS) on the T cell by the major histocompatibility complex (MHCII) peptide and co-stimulatory molecules on the APC. Both are required for production of an effective immune response; in the absence of co-stimulation, T cell receptor signaling alone results in anergy. The signaling pathways downstream from co-stimulatory molecules usually engages the PI3K pathway generating PIP3 at the plasma membrane and recruiting PH domain containing signaling molecules like PDK1 that are essential for the activation of PKC-θ, and eventual IL-2 production. Optimal CD8+ T cell response relies on CD4+ signaling. CD4⁺ cells are useful in the initial antigenic activation of naive CD8 T cells, and sustaining memory CD8+ T cells in the aftermath of an acute infection. Therefore, activation of CD4+T cells can be beneficial to the action of CD8+T cells.

According to a specific embodiment, the antibody is a homolog of any of the antibodies of Table A above comprising an amino acid sequence at least 90%, 91%, 92%, 93%, 94%, 95%, 96%, 97% 98% or 99% identical to CDRs of the VH chain and/or VL chain, as long as it is capable of binding TREM2 and preferably inhibiting its activity as evidenced by activation of macrophages.

According to a specific embodiment, the antibody is a homolog of any of the antibodies of Table B above comprising an amino acid sequence at least 90%, 91%, 92%, 93%, 94%, 95%, 96%, 97% 98% or 99% identical to CDRs of the VH chain and/or VL chain, as long as it is capable of binding Gpnmb and preferably inhibiting its activity as evidenced by activation of CD4 T cells.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of and a non-conservative substitution is given a score of zero, a conservative substitution is given a score between zero and. The scoring of conservative substitutions is calculated, e.g., according to the algorithm of Henikoff S and Henikoff JG. [Amino acid substitution matrices from protein blocks. Proc. Natl. Acad. Sci. U.S.A. 992, 89(22): 095-9]. Identity (e.g., percent homology) can be determined using any homology comparison software, including for example, the BlastN or BlastP software of the National Center of Biotechnology Information (NCBI) such as by using default parameters.

When referring to “at least 90% identity” the claimed invention also refer to at least 9%, at least 92%, at least 93%, at least 94%, at least 95%, at least 96%, at least 97%, at least 98%, at least 98% or 00% identity where each represents a different embodiment.

According to a specific embodiment, the level of identity is at least 90% over the entire sequence (any of the VH and/or VL chains described herein) such as determined as described herein.

According to a specific embodiment, the level of identity is at least 90%, 9%, 92%, 93%, 94%, 95%, 96%, 97%, 98%, 99% over at least one (or at least 2, 3, 4 or 5) of the CDR sequences of an antibody of Table A or B as described herein.

Exemplary CDR sequences and complete light and heavy chains of human antibodies are provided in Table A or B above.

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

In a particular embodiment, the antibody is a monoclonal antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference and the Examples section which follows).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

According to one embodiment, the antibody is a monospecific antibody.

According to one embodiment, the antibody is a bispecific antibody recognizing two different antigens, TREM2 and Gpnmb, a multivariant antibody or a chimeric antibody.

“Bispecific antibody” of the present invention has two different antigen binding sites, such that the antibody specifically binds to two different antigens. Such antibodies may be generated by combining parts of two separate antibodies or antibody fragments that recognize two different antigenic groups or modifying a single antibody molecule to comprise two specificities (as discussed in detail hereinabove).

According to one embodiment, the bi-specific antibody is a hybrid antibody having two different heavy/light chain pairs and two different binding sites.

According to one embodiment, the bi-specific antibody comprises an antigen recognition domain in a structural loop region of the antibody (e.g. CH3 region of the heavy chain). Accordingly, the bi-specific antibody may comprise an antibody fragment comprising a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Antibodies having higher valencies (i.e., the ability to bind to more than two antigens) can also be prepared; they are referred to as multispecific antibodies.

According to a specific embodiment, the bispecific antibody comprises in at least one arm thereof the antigen recognition domain of any one of the antibodies described hereinabove or specifically in the CDRs of the antibodies of Table A or B.

According to a specific embodiment, the antibody comprises in one arm thereof an anti TREM2 antibody and in another arm an anti Gpmnb antibody.

According to a specific embodiment, the antibody comprises in one arm thereof the anti TREM2 antibody as described herein and in another arm the anti Gpmnb antibody as described herein.

In order to produce the multispecific antibody of some embodiments of the invention, the present moieties can be modified at the Fc region e.g., the CH3 domain (according to kabat) as well known in the art. Such a modification ensures correct assembly of the multispecific antibody via the heavy chains.

Accordingly, the CH3 domain of one heavy chain is altered, so that within the original interface the CH3 domain of one heavy chain that meets the original interface of the CH3 domain of the other heavy chain within the multispecific antibody, an amino acid residue is replaced with an amino acid residue having a larger side chain volume, thereby generating a protuberance within the interface of the CH3 domain of one heavy chain which is positionable in a cavity within the interface of the CH3 domain of the other heavy chain; and the CH3 domain of the other heavy chain is altered, so that within the original interface of the second CH3 domain that meets the original interface of the first CH3 domain within the trivalent, bispecific antibody an amino acid residue is replaced with an amino acid residue having a smaller side chain volume, thereby generating a cavity within the interface of the second CH3 domain within which a protuberance within the interface of the first CH3 domain is positionable (also known as “the knobs-into-holes” approach by Genentech).

According to a specific embodiment, the amino acid residue having a larger side chain volume is selected from the group consisting of arginine (R), phenylalanine (F), tyrosine (Y), tryptophan (W).

According to a specific embodiment, the amino acid residue having a smaller side chain volume is selected from the group consisting of alanine (A), serine (S), threonine (T), valine (V).

According to a specific embodiment, both CH3 domains are further altered by the introduction of cysteine (C) as amino acid in the corresponding positions of each CH3 domain such that a disulfide bridge between both CH3 domains can be formed.

In a specific embodiment, the bispecific comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M., et al., Nature Biotech 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the bispecific antibody comprises Y349C, T366W mutations in one of the two CH3 domains and E356C, T366S, L368A, Y407V mutations in the other of the two CH3 domains or the bispecific antibody comprises Y349C, T366W mutations in one of the two CH3 domains and S354C, T366S, L368A, Y407V mutations in the other of the two CH3 domains (the additional Y349C mutation in one CH3 domain and the additional E356C or S354C mutation in the other CH3 domain forming a interchain disulfide bridge) (numbering always according to EU index of Kabat). But also other knobs-in-holes technologies as described by EP 1 870 459A1, can be used alternatively or additionally. A specific example for the bispecific antibody are R409D; K370E mutations in the CH3 domain of the “knobs chain” and D399K; E357K mutations in the CH3 domain of the “hole chain” (numbering always according to EU index of Kabat).

In another embodiment the bispecific antibody comprises a T366W mutation in the CH3 domain of the “knobs chain” and T366S, L368A, Y407V mutations in the CH3 domain of the “hole chain” and additionally R409D; K370E mutations in the CH3 domain of the “knobs chain” and D399K; E357K mutations in the CH3 domain of the “hole chain”.

In another embodiment the bispecific antibody comprises Y349C, T366W mutations in one of the two CH3 domains and S354C, T366S, L368A, Y407V mutations in the other of the two CH3 domains or the bispecific antibody comprises Y349C, T366W mutations in one of the two CH3 domains and S354C, T366S, L368A, Y407V mutations in the other of the two CH3 domains and additionally R409D; K370E mutations in the CH3 domain of the “knobs chain” and D399K; E357K mutations in the CH3 domain of the “hole chain”.

According to a specific embodiment, Y349C/T366S/L368A/Y407V mutations are introduced for the 1st mAb (e.g., anti TREM2) and S354C/T366W for the 2nd mAb (e.g., anti Gpnmb) (Merchant et al., 1998; Ridgway et al., 1996).

Alternatively or additionally, for correct heavy-light chain pairing, at least one of the moieties can be expressed in the CrossMab format (CH1-CL swapping).

The basis of the CrossMab technology is the crossover of antibody domains within one arm of a bispecific IgG antibody enabling correct chain association, whereas correct heterodimerization of the heavy chains can be achieved by the knob-into-hole technology as described above or charge interactions. This can be achieved by exchange of different domains within a Fab-fragment. Either the Fab domains (in the CrossMabFab format), or only the variable VH-VL domains (CrossMab^{VH-VL} format) or the constant CH1-CL domains (CrossMab^{CH1-CL} format) within the Fab-fragment can be exchanged for this purpose. Indeed, for the CrossMab^{CH1-CL} format the respective original light chain and the novel VL-CH1 light chain do not result in undesired interactions with the respective original and VH-CL containing heavy chains, and no theoretical side products can be formed. In contrast, in the case of the CrossMab^{Fab} format a non-functional monovalent antibody (MoAb) as well as a non-functional Fab-fragment can be formed. These side products can be removed by chromatographic techniques. In the case of the CrossMab^{VH-VL} format an undesired side product with a VL-CH1/VL-CL domain association known from Bence-Jones proteins can occur between the VL-CH1 containing heavy chain and the original unmodified VL-CL light chain. The introduction of repulsive charge pairs based on existing conserved charge pairs in the wildtype antibody framework into the constant CH1 and CL domains of the wildtype non-crossed Fab-fragment can overcome the formation of this Bence-Jones-like side product in the CrossMab^{VH-VL+/-} format. More details on CrossMab Technology can be found in Klein et al. Methods 154, 1 Feb. 2019, Pages 21-31c.

Alternatively, multispecific e.g., bispecific antibodies described herein can be prepared by conjugating the moieties using methods known in the art. For example, each moiety of the multispecific antibody can be generated separately and then conjugated to one another. A variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-malcimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. (USA) 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

Alternatively or additionally, the conjugation of each moiety of the multispecific antibody can be done via sulfhydryl bonding of the C-terminus hinge regions of the two heavy chains. In a specific embodiment, the hinge region is modified to contain an odd number of sulfhydryl residues, preferably one, prior to conjugation.

According to an aspect of the invention there is provided a method of producing an antibody, the method comprising:
(a) expressing in a host cell a heterologous polynucleotide encoding the antibody as described herein; and optionally
(b) recovering the antibody from the host cell.

Thus, a polynucleotide encoding an antibody of some embodiments of the invention is cloned into an expression construct selected according to the expression system used. Exemplary polynucleotide sequences are provided in SEQ ID NOs: 6-23, 42-59; 186-216, 248-278.

A variety of prokaryotic or eukaryotic cells can be used as host-expression systems to express the antibody of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the antibodies of some embodiments of the invention.

Examples for mammalian expression vectors include, but are not limited to, pcDNA3, pcDNA3.(+/−), pGL3, pZcoSV2(+/−), pSccTag2, pDisplay, pEF/myc/cyto, pCMV/myc/cyto, pCR3., pSinRep5, DH26S, DHBB, pNMT, pNMT4, pNMT8, which are available from Invitrogen, pCI which is available from Promega, pMbac, pPbac, pBK-RSV and pBK-CMV which are available from Strategene, pTRES which is available from Clontech, and their derivatives.

According to a specific embodiment, the vectors used are pFUSE2-CLIg-mk, pFUSE2-CHIg-mG1 for light and heavy chains respectively. According to a specific embodiment, the antibodies are transiently expressed in Expi293F cells.

Expression vectors containing regulatory elements from eukaryotic viruses such as retroviruses can be also used. SV40 vectors include pSVT7 and pMT2. Vectors derived from bovine papilloma virus include pBV-MTHA, and vectors derived from Epstein Bar virus include pHEBO, and p205. Other exemplary vectors include pMSG, pAV009/A+, pMTO0/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Examples of bacterial constructs include the pET series of E. coli expression vectors [Studier et al. (990) Methods in Enzymol. 85:60-89).

In yeast, a number of vectors containing constitutive or inducible promoters can be used, as disclosed in U.S. Pat. No: 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 9S RNA promoters of CaMV [Brisson et al. (984) Nature 30:5-54], or the coat protein promoter to TMV [Takamatsu et al. (987) EMBO J. 6:307-3] can be used. Alternatively, plant promoters such as the small subunit of RUBISCO [Coruzzi et al. (984) EMBO J. 3:- 680 and Brogli et al., (984) Science 224:838-843] or heat shock promoters, e.g., soybean hsp7.5-E or hsp7.3-B [Gurley et al. (986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 988, Methods for Plant Molecular Biology, Academic Press, NY, Section VIII, pp 42-463.

Other expression systems such as insects and mammalian host cell systems which are well known in the art and are further described hereinbelow can also be used by some embodiments of the invention.

It will be appreciated that antibodies can also be produced in in-vivo systems such as in mammals, e.g., goats, rabbits etc.

Recovery of the recombinant antibody is effected following an appropriate time (in culture). The phrase “recovering the antibody” refers to collecting the whole fermentation medium containing the antibody and need not imply additional steps of separation or purification. Notwithstanding the above, antibodies of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Once antibodies are obtained, they may be tested for activity, such as described above.

In some embodiments an antibody described herein includes modifications to improve its ability to mediate effector function. Such modifications are known in the art and include afucosylation, or engineering of the affinity of the Fc towards an activating receptor, mainly FCGR3a for ADCC, and towards Clq for CDC. Table B of U.S. 10. 428,143 summarizes various designs reported in the literature for effector function engineering.

Methods of producing antibodies with little or no fucose on the Fe glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 Dec.; 20 (12): 1607-18. Another approach to obtaining antibodies with lowered levels of fucosylation can be found in U.S. Pat. No. 8,409,572, which teaches selecting cell lines for antibody production for their ability to yield lower levels of fucosylation on antibodies Antibodys can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

According to another specific embodiment, the antibody has an Fc domain which has null or no effector function. The IgG1 isoform of human antibodies is known in the art to have little or no ADCC or CDC activity.

Though other isotypes are also contemplated e.g., IgG2, IgG3 or IgG4.

The antibody may be soluble or non-soluble.

Non-soluble antibodies may be a part of a particle (synthetic or non-synthetic, e.g., liposome) or a cell (e.g., CAR-T cells, in which the antibody is part of a chimeric antigen receptor (CAR) typically as an scFv fragment).

Thus, in some embodiments, antibody sequences of the invention may be used to develop a chimeric antigen receptor (CAR). CARs are transmembrane receptors expressed on immune cells that facilitate recognition and killing of target cells (e.g. myeloid cells expressing TREM2). CARs typically include three basic parts. These include an ectodomain (also known as the recognition domain), a transmembrane domain and an intracellular (signaling) domain. Ectodomains facilitate binding to cellular antigens on target cells, while intracellular domains typically include cell signaling functions to promote the killing of bound target cells. Further, they may have an extracellular domain with one or more of the antibody variable domains described herein or fragments thereof. CARs of the invention also include a transmembrane domain and cytoplasmic tail. CARs may be designed to include one or more segments of an antibody, antibody variable domain and/or antibody CDR, such that when such CARs are expressed on immune effector cells, the immune effector cells bind and clear any cells that are recognized by the antibody portions of the CARs.

Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

CARs engineered to target tumors have specificity for TREM2 according to some embodiments of the invention. In some embodiments, ectodomains of these CARs may include one or more antibody variable domains or a fragment thereof. In some embodiments, CARs are expressed in T cells, and may be referred to as “CAR-engineered T cells” or “CAR-Ts”. CAR-Ts may be engineered with CAR ectodomains having one or more antibody variable domains.

Thus, in some embodiments of the present disclosure, antibody sequences of the invention may be used to develop a chimeric antigen receptor (CAR). In some embodiments, CARs are transmembrane receptors expressed on immune cells that facilitate recognition and killing of target cells such as myeloid cells expressing TREM2 (e.g. as exemplified for TREM2 expressing HEK293 cell, humanized TREM2 Bone marrow derived macrophage (BMDM), human monocyte derived macrophage (hMac) line).

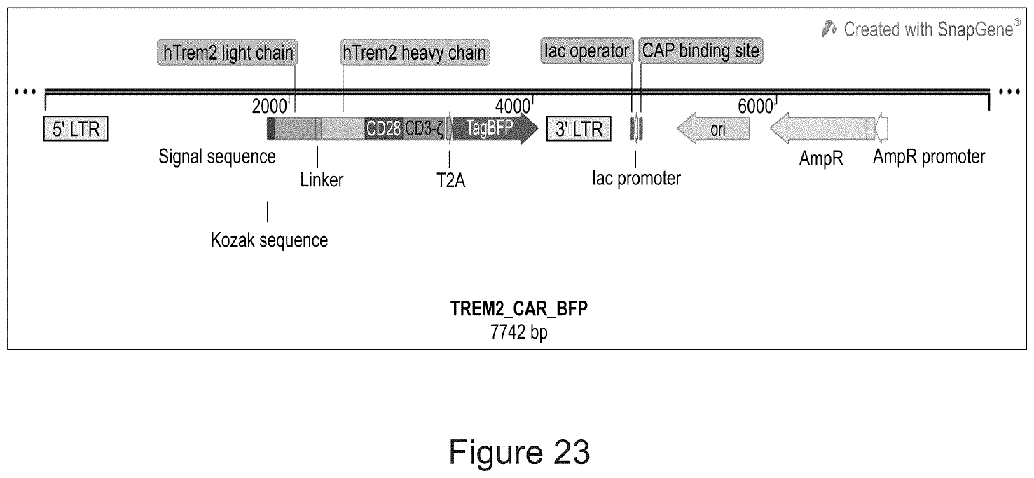

Immune cells expressing the CARs of the invention (see for example FIG. 23) can be generated by well-known techniques, such as set forth in the examples, immune cells expressing the CARs of the invention can be used to kill TREM2 expressing myeloid cells. Thus, the invention encompasses methods of killing TREM2 expressing myeloid cells comprising contacting a population of cells comprising TREM2 expressing myeloid cells with immune cells, preferably T cells, comprising a CAR of the invention, wherein TREM2 expressing myeloid cells are killed.

In one embodiment, contacting is effected ex vivo or in vitro.

Thus, for example, following the collection of immune effector cells from a subject in need thereof, the cells may be genetically engineered to express the disclosed CARs. Subsequently, the genetically engineered cells can be infused back into the subject.

The disclosed CAR-modified immune effector cells may be used or administered either alone, or as a pharmaceutical composition in combination with diluents and/or with other components such as IL-2, IL-15, IL-21, or other cytokines or cell populations. These components can be added as a protein constituent or expressed by the CAR-modified immune effector cells, for example, by genetically engineering them to express the disclosed cytokines.

Pharmaceutical compositions can comprise CAR-modified immune effector cells as described herein, in combination with one or more pharmaceutically or physiologically acceptable carriers, diluents or excipients.

In in some embodiments, compositions for use in the disclosed methods are formulated for intravenous administration. Pharmaceutical compositions may be administered in any manner appropriate to kill TREM2 expressing myeloid cells. The quantity and frequency of administration will be determined by such factors as the condition of the patient, and the severity of the patient's disease, although appropriate dosages may be determined by clinical trials.

In various embodiments, a therapeutic amount of CAR-modified immune effector cells is administered to a patient. A “therapeutic amount” can be determined by a physician with consideration of individual differences in age, weight, tumor size, extent of infection or metastasis, and condition of the patient (subject). A pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. Increasing the cytotoxic activity or therapeutic activity of an antibody where necessary can also be achieved such as by using an antibody-drug conjugate (ADC) concept. In such a configuration the antibody is attached to a heterologous effector moiety that can be used to increase its toxicity or to render it detectable.

In some embodiments, antibodies of the invention may be developed for antibody drug conjugate (ADC) therapeutic applications. ADCs are antibodies in which one or more cargo (e.g., therapeutic agents) are attached [e.g. directly or via linker (e.g. a cleavable linker or a non-cleavable linker)]. ADCs are useful for delivery of therapeutic agents (e.g., drugs or cytotoxic agents) to one or more target cells or tissues (Panowski, S. et al., 204. mAbs 6:, 34-45). In some cases, ADCs may be designed to bind to a surface antigen on a targeted cell. Upon binding, the entire antibody-antigen complex may be internalized and directed to a cellular lysosome. ADCs may then be degraded, releasing the bound cargo.

The therapeutic agent may be a small molecule drug, a proteinaceous agent (e.g., cytokine or chemokine, e.g., tumor necrosis factor (TNF) or IL12), a nucleic acid agent, radio-isotopes and carbohydrate and the like. These can serve as cytotoxic agents, e.g., chemotherapy.

According to a specific embodiment, the therapeutic agent is a nucleic acid sequence (e.g., DNA or RNA, e.g., mRNA) which codes for a viral antigen, in order to elicit an anti viral immune response against the tumor. Examples of viral antigens include, but are not limited to CMV antigens, EBV antigens, Coronavirus antigens and the like.

As used herein, the term “cytotoxic agent” refers to refers to a substance that inhibits or prevents the function of cells and/or causes destruction of cells.

Where the cargo is a cytotoxic agent, the target cell will be killed or otherwise disabled. Cytotoxic agents may include, but are not limited to cytoskeletal inhibitors [e.g., tubulin polymerization inhibitors, and kinesin spindle protein (KSP) inhibitors], DNA damaging agents (e.g., calicheamicins, duocarmycins, and pyrrolobenzodiazepine dimers such as talirine and tesirine), topoisomerase inhibitors [e.g., camptothecin compounds or derivatives such as 7-ethyl-O-hydroxycamptothecin (SN-38) and exatecan derivative DXd], transcription inhibitors (e.g., RNA polymerase inhibitors such as amanitin), and kinase inhibitors [e.g., phosphoinositide 3-kinase (PI3K) inhibitors or mitogen-activated protein kinase kinase (MEK) inhibitors].

Tubulin polymerization inhibitors may include, but are not limited to, maytansines (e.g., emtansine [DM] and ravtansine [DM4]), auristatins, tubulysins, and vinca alkaloids or derivatives thereof. Exemplary auristatins include auristatin E (also known as a derivative of dolastatin-0), auristatin EB (AEB), auristatin EFP (AEFP), monomethyl auristatin E (MMAE), monomethyl auristatin F (MMAF), auristatin F and dolastatin. Exemplary tubulysin compounds include naturally occurring tubulysins A, B, C, D, E, F, G, H, I, U, and V, and tubulysin analogs such as pretubulysin D (PTb-D43) and N.sup.4-desacetoxytubulysin H (Tbl). Exemplary vinca alkaloids include vincristine, vinblastine, vindesine, and navelbine (vinorelbine). In some embodiments, cytotoxic agents may include auristatin derivatives [e.g. -aminopropan-2-yl-auristatin F, auristatin F-hydroxypropylamide, auristatin F-propylamide, auristatin F phenylenediamine (AFP)]; tubulysin derivatives; vinca alkaloid derivatives [e.g. N-(3-hydroxypropyl)vindesine (HPV)], and any of those described in U.S. Pat. Nos. 8,524,24; 8,685,383; 8,808,9; and 9,254,339; US Pat. Application Publications US205034008A, US2060220696A and US2060022829A; the contents of each of which are herein incorporated by reference in their entirety.

The term is intended to also include radioactive isotopes (e.g., ²¹¹At, ¹³¹I, ¹²⁵I, ³²P, ³⁵S and radioactive isotopes of Lu, including ¹⁷⁷Lu, ⁸⁶Y, ⁹⁰Y, ¹¹¹In, ¹⁷⁷Lu, ²²⁵ Ac, ²¹²Bi, ²¹³Bi, ⁶⁶Ga, ⁶⁷Ga, ⁶⁸Ga, ⁶Cu, ⁶⁷Cu, ⁷¹ As, ⁷²As, ⁷⁶As, ⁷⁷As, ⁶⁵Zn, ⁴⁸V, ²⁰³Pb, ²⁰⁹Pb, ²¹²Pb, ¹⁶⁶Ho, ¹⁴⁹Pm, ¹⁵³Sm, ²⁰¹TI, ¹⁸⁸Re, ¹⁸⁶Rc and ⁹⁹mTc), enzymes and fragments thereof such as nucleolytic enzymes, antibiotics, therapeutic RNA molecules (e.g., siRNA, antisense oligonucleotides, microRNA, ribozymes, RNA decoys, aptamers), DNAzymes, and toxins such as small molecule toxins or enzymatically active toxins of bacterial, fungal, plant or animal origin, such as pokeweed antiviral protein (PAP), ricin toxin A, abrin, gelonin, saporin, cholera toxin A, diphtheria toxin, Pseudomonas exotoxin, and alpha-sarcin, including fragments and/or variants thereof.

In some embodiments, antibody-drug conjugates (ADCs) of the invention may further comprise one or more polymeric carrier connecting the antibody and the therapeutic agents (e.g., antibody-polymer-drug conjugates). As used herein, the term “polymeric carrier” refers to a polymer or a modified polymer, which may be covalently attached to one or more therapeutic agents and/or antibodies. Polymeric carriers may provide additional conjugation sites for therapeutic agents, increasing the drug-to-antibody ratio and enhancing therapeutic effects of ADCs. In some embodiments, polymeric carriers used in this invention may be water soluble and/or biodegradable. Such polymeric carriers may include, but are not limited to poly(ethylene glycol) (PEG), poly(N-(2-hydroxypropyl)methacrylamide) (polyHPMA), poly(.alpha.-amino acids) [e.g., poly(L-lysine), poly(L-glutamic acid), and poly((N-hydroxyalky)glutamine)], carbohydrate polymers [e.g., dextrins, hydroxyethylstarch (HES), and polysialic acid], glycopolysaccharides (e.g., homopolysaccharide such as cellulose, amylose, dextran, levan, fucoidan, carraginan, inulin, pectin, amylopectin, glycogen and lixenan; or homopolysaccharide such as agarose, hyluronan, chondroitinsulfate, dermatansulfate, keratansulfate, alginic acid and heparin), glycolipids, glycoconjugates, polyglycerols, polyvinyl alcohols, poly(acrylic acid), polyketal and polyacetal [e.g., poly(l-hydroxymethylethylene hydroxymethylformal), also known as PHF or FLEXIMER.RTM., described in U.S. Pat. Nos. 5,8,50; 5,863,990; and 5,958,398; the contents of each of which are herein incorporated by reference in their entirety], and derivatives, dendrimers, copolymers and mixtures thereof. For example, the polymeric carrier may include a copolymer of a polyacetal/polyketal (e.g., PHF) and a hydrophilic polymer such as polyacrylates, polyvinyl polymers, polyesters, polyorthoesters, polyamides, polypeptides, and derivatives thereof.

In some embodiments, therapeutic agents are attached (e.g., covalently bonded) to antibodies of the invention directly or via linkers. In some embodiments, therapeutic agents are attached to polymeric carriers directly or via linkers, and the polymeric carriers are attached to the antibodies directly or via linkers. In some embodiments, linkers may comprise an oxalic, malonic, succinic, glutaric, adipic, pimelic, suberic, azelaic, sebacic, phthalic, isophthalic, terephthalic, diglycolic acid, tartaric, glutamic, fumaric, or aspartic moiety, including amide, imide, or cyclic-imide derivatives of each thereof, and each optionally substituted. Exemplary linkers may include any of those disclosed in U.S. Pat. Nos. 8,524,24; 8,685,383; 8,808,9; 9,254,339; and/or 9,555,2 the contents of each of which are herein incorporated by reference in their entirety.

In some embodiments, linkers may be cleavable linkers. Cleavable linkers may break down under certain conditions (such as changes in pH, temperature, or reduction) or cleaved by enzymes (e.g., proteases and glucuronidases) to allow release of therapeutic agents from ADCs. Such linkers may include a labile bond such as an ester bond, amide bond, or disulfide bond. Non-limiting cleavable linkers may include pH-sensitive linkers (e.g., hydrazone, semicarbazone, thiosemicarbazone, cis-aconitic amide, thioether, orthoester, acetal, or ketal); reduction-sensitive linkers [e.g., N-succinimidyl 3-(2-pyridyldithio)propionate (SPDP), N-succinimidyl 4-(2-pyridyldithio)butanoate (SPDB), N-succinimidyl 4-(2-pyridyldithio)pentanoate (SPP), N-succinimidyl-S-acetylthioacetate (SATA) and N-succinimidyl-oxycarbonyl-alpha-methyl-alpha-(2-pyridyl-dithio)toluene or 2,5-dioxopyrrolidin—yl 4-(-(pyridin-2-yldisulfanyl)ethyl)benzoate (SMPT)]; photosensitive linkers; and enzymatically cleavable linkers [e.g., peptide linkers such as valine-citrulline, valine-citrulline-p-aminobenzoyloxycarbonyl (vc-PAB), malcimidocaproyl-valine-citrulline-p-aminobenzoyloxycarbonyl (MC-vc-PAB), linkers cleavable by glucuronidases, such as glucuronide-MABC, or linkers cleavable by esterases].

In other embodiments, linkers may be non-cleavable linkers. Non-cleavable linkers may increase plasma stability of the ADCs compared to cleavable linkers. Exemplary non-cleavable linkers include maleimide alkane and maleimide cyclohexane (MCC).

Antibody-drug conjugates (ADCs) of the invention may be prepared using any method known in the art. For example, therapeutic agents may be modified to contain a functional group that can react with a functional group on the antibody. Antibody-drug conjugates (ADCs) may be prepared by reacting the two functional groups to form a conjugate. In some cases, polymeric carriers may be modified to contain functional groups that can react with the functional group on the therapeutic agents and the functional group on the antibody under different chemical conditions. Antibodies, polymeric carriers, and therapeutic agents may be linked to form the antibody-polymer-drug conjugates through sequential chemical reactions. Conjugation to antibodies may employ a lysine or a cysteine residue as the conjugation site. In some embodiments, antibodies may be engineered to have additional lysine or cysteine residues. Such approaches may avoid disruption of antibody structure (e.g., interchain disulfide bonds) and maintain antibody stability and/or activity.

Alternatively or additionally, various agents can be used to increase the therapeutic efficacy of the antibodies. Such as for example combining the antibodies with pro-inflammatory cytokines such as of the TNF family or IL12, e.g., IFNα, IFNβ, IFNγ, IL-2, IL-11, IL-21, G-CSF, GM-CSF, and/or TNFα.

According to a specific embodiment, the cytokine is conjugated to the antibody.

According to a specific embodiment, the conjugation is covalent.

According to a specific embodiment, the conjugate is a chimeric protein in which the antibody is translationally fused to the cytokine (upstream or downstream thereto with or without a linker, as described herein).

According to a specific embodiment, the cytokine is IL-2.

According to a specific embodiment, the conjugate is as set forth in SEQ ID NO: 496 and 498 or 497 and 499.

According to a specific embodiment, the cytokine is IL-15.

The invention encompasses uses and methods of enhancing myeloid reprogramming with the antibodies of the invention.

In one embodiment, the invention encompasses methods of enhancing myeloid reprogramming comprising contacting a population of TREM2-expressing cells with an antibody of the invention and a cytokine, such as a cytokine selected from the TNF family, IL-21, IL-12, IL-15, IFNα, IFNβ, IFNγ, IL-2, IL-11, G-CSF, GM-CSF, and/or TNFα. The method can enhance myeloid reprogramming of the population of TREM2-expressing cells. As detailed in the examples, the method can provide a synergistic effect of the antibody with the cytokine.

Chimeric proteins in which an antibody of the invention is translationally fused to a cytokine, such as a cytokine selected from the TNF family, IL-12, IL-15, IFNα, IFNß, IFNγ, IL-2, IL-11, IL-21, G-CSF, GM-CSF, and/or TNF, can be generated by well-known techniques, such as set forth in the examples. The chimeric proteins can be used to enhance myeloid reprogramming. In one embodiment, the invention encompasses methods of enhancing myeloid reprogramming comprising contacting a population of TREM2-expressing cells with a chimeric protein of the invention. As detailed in the examples, the method can provide a synergistic effect of the chimeric protein.

Antibodies of some embodiments of the invention are endowed with an immune-modulatory activity.

Thus, according to an aspect of the present invention there is provided a method of reducing the immune suppressor activity of myeloid cells, the method comprising contacting myeloid cells with an effective amount of the antibody or antibody fragment or bispecific antibody as described herein, thereby reducing the immune suppressor activity of myeloid cells.

According to another aspect, there is provided a method of activating CD4 T cells, the method comprising contacting CD4 T cells with an effective amount of the antibody or fragment thereof, thereby activating the CD4 T cells.

According to an embodiment, the contacting is effected in vivo.

According to another embodiment, the contacting is effected ex vivo.

The term “myeloid cells” as used herein refers to cells which arise from the common myeloid progenitor (CMP). In one embodiment, myeloid cells are ones which, arise from the lineage of the myeloblast and their daughter types (e.g. basophils, neutrophils, eosinophils, monocytes and macrophages). One subgroup of myeloid cells are immune suppressor myeloid cells.

According to a specific embodiment, the myeloid cells are M2 macrophages which acquire an M1 phenotype upon incubation with antibodies (to TREM2) of some embodiments of the invention.

As mentioned, an antibody/antibodies are contacted with myeloid cells of the subject in order to reduce the amount and/or activity of a specific subpopulation of said myeloid cells—those expressing both TREM2 and Gpnmb.

In one embodiment, the contacting is carried out in vivo.

In another embodiment, the contacting is carried out ex vivo—i.e. myeloid cells are removed from a subject and subsequently contacted with the agent.

Myeloid cells are typically removed from subjects by bone marrow biopsy. Mobilizing agents such as Plerixafor® and G-CSF, can be used to mobilize the cells to the periphery.

The antibody of this aspect of the present invention specifically increases the activity of macrophages expressing both Triggering Receptor Expressed On Myeloid Cells 2 (TREM2) and Transmembrane glycoprotein NMB (Gpnmb).

In one embodiment, the antibody/ies enhances (activated macrophages) the activity of cells expressing both markers at least 2 fold compared to cells expressing only one of the markers (i.e. only TREM2 and not Gpnmb or vice versa). In another embodiment, the antibody/ies enhances the activity of cells expressing both markers at least 5 fold compared to cells expressing only one of the markers (i.e. only TREM2 and not Gpnmb or vice versa). In another embodiment, the antibody/ies enhances the activity of cells expressing both markers at least 10 fold compared to cells expressing only one of the markers (i.e. only TREM2 and not Gpnmb or vice versa).

Since the method described herein is used to reduce the immune suppressor activity of myeloid cells, the present inventors conceive that the method may be used to treat cancer.

Thus, according to another aspect of the present invention there is provided a method of treating cancer in a subject in need thereof, the method comprising administering to the subject an effective amount of the antibody, antibody fragment, combination thereof (anti TREM2 and anti Gpnmb) or bispecific antibody as described herein, thereby treating the cancer.

According to another aspect there is provided a method of treating cancer in a subject in need thereof, the method comprising:
(a) reducing the immune suppressor activity of myeloid cells according to the method as described hereinabove, wherein the myeloid cells are derived from the subject; and subsequently
(b) transplanting the myeloid cells to the subject, thereby treating the cancer.

As used herein “reducing” refers to at least 10%, 20%, 30%, 30%, 40%, 50%, 60%, 70%, 80%, 90%, 2 fold, 3 fold, 5 fold, 10 fold lower immune suppressor activity in the presence of the antibody, as compared to a control (negative, e.g., untreated cells) sample.

As used herein “subject” refers to a mammal, e.g., human, diagnosed with cancer.

The terms “cancer” and “cancerous” refer to or describe the physiological condition in mammals that is typically characterized by unregulated malignant cell growth.

Examples of cancers that can be analyzed and treated according to some embodiments of the invention, include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to a specific embodiment, the cancer is melanoma.

According to a specific embodiment, the cancer is a solid tumor (lung cancer, liver cancer, ovarian cancer, gastric cancer and breast cancer).

According to a specific embodiment, the cancer is a primary tumor.

According to a specific embodiment, the cancer is metastatic.

According to a specific embodiment, the cancer is a secondary tumor.

According to a specific embodiment, the lung cancer is non-small cell lung cancer.

According to a specific embodiment, the lung cancer is small cell lung cancer.

According to a specific embodiment, the liver cancer is Hepatocellular carcinoma.

According to another aspect of the present invention there is provided a method of treating cancer in a subject in need thereof, the method comprising administering to the subject a therapeutically effective amount of
(i) a first antigen recognition domain which down-regulates the activity of TREM2; and
(ii) a second antigen recognition domain which specifically down-regulates the activity of Gpnmb, thereby treating the cancer.

According to one embodiment, the first antigen recognition domain binds specifically to TREM2 which is expressed on myeloid cells. According to another embodiment, the second antigen recognition domain binds specifically to Gpnmb. The phrase “specifically bind(s)” or “bind(s) specifically” when referring to a binding molecule refers to a binding molecule which has intermediate or high binding affinity, exclusively or predominately, to a target molecule, such as to TREM2 or Gpnmb. The phrase “specifically binds to” refers to a binding reaction which is determinative of the presence of a target protein (such as TREM2 or Gpnmb) in the presence of a heterogeneous population of proteins and other biologics. Thus, under designated assay conditions, the specified binding molecules bind preferentially to a particular target protein (e.g. TREM2 or Gpnmb) and do not bind in a significant amount to other components present in a test sample. Specific binding to a target protein under such conditions may require a binding molecule that is selected for its specificity for a particular target protein. A variety of assay formats may be used to select binding molecules that are specifically reactive with a particular target protein. For example, solid-phase ELISA immunoassays, immunoprecipitation, Biacore and Western blot may be used to identify binding molecules that specifically bind to TREM2 or Gpnmb. Typically, a specific or selective reaction will be at least twice background signal or noise and more typically more than 10 times background. Given that the binding molecule is an antibody, the phrase “specifically binds to” refers to a binding reaction that is determinative of the presence of the antigen (such as TREM2 or Gpnmb) in a heterogeneous population of proteins and other biologics. Typically, an agent that specifically binds to an antigen binds the antigen with a dissociation constant (K_{D}) of at least about 1×10⁻⁶ to 1×10⁻⁷, or about 1×10⁻⁸ to 1× 10⁻⁹ M, or about 1×10⁻¹⁰ to 1×10⁻¹¹ or higher; and/or binds to the predetermined antigen (e.g. of TREM2 or Gpnmb) with an affinity that is at least two-fold, five-fold, ten-fold, twenty-fold greater than its affinity for binding to a non-specific antigen (e.g., BSA, casein) other than the predetermined antigen or a closely-related antigen.

According to a particular embodiment, the antigen recognition domain which decreases the amount and/or activity of TREM2 is an inhibitor antibody, also referred to herein as an antagonist antibody.

According to a specific embodiment, the affinity of the selected antibodies is in the range of 10⁻⁸ M-10⁻¹⁴ M, such as determined by a surface plasmon resonance (SPR) assay (see conditions described in the Examples section).

According to some embodiments, the affinity range is 10⁻⁸ M-10⁻¹⁴ M.

According to some embodiments, the affinity range is 10⁻⁸ M-10⁻¹³ M.

According to some embodiments, the affinity range is 10⁻⁸ M-10⁻¹² M.

According to some embodiments, the affinity range is 10⁻⁸ M-10⁻¹¹ M.

According to some embodiments, the affinity range is 10⁻⁸ M-10⁻¹⁰ M.

According to some embodiments, the affinity range is 10⁻⁸ M-10⁻¹⁹ M.

According to some embodiments, the affinity range is 10⁻⁹ M-10⁻¹⁴ M.

According to some embodiments, the affinity range is 10⁻⁹ M-10⁻¹³ M.

According to some embodiments, the affinity range is 10⁻⁹ M-10⁻¹² M.

According to some embodiments, the affinity range is 10⁻⁹ M-10⁻¹¹ M.

According to some embodiments, the affinity range is 10⁻⁹ M-10⁻¹⁰ M.

According to some embodiments, the affinity range is 10⁻¹⁰ M-10⁻¹³ M.

According to some embodiments, the affinity range is 10⁻¹⁰ M-10⁻¹² M.

According to some embodiments, the affinity range is 10⁻¹⁰ M-10⁻¹¹ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (ka) and the dissociation rate constant (kd) can be measured. The equilibrium dissociation constant (KD in M) can then be calculated from the ratio of the kinetic rate constants (kd/ka). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (KD in M) and the association rate constant (ka in M′V1) can be measured. The dissociation rate constant (kd) can be calculated from these values (KD X ka). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (ka and kd) and affinity (KD) constants can be calculated in real-time using the bio-layer interferometry method. In some embodiments, the antigen binding proteins described herein exhibit desirable characteristics such as binding avidity as measured by kd (dissociation rate constant) for human TREM2 and for human Gpnmb of about 10⁻², 10⁻³, 10⁻⁴, 10⁻⁵, 10⁻⁶ or lower (lower values indicating higher binding avidity), and/or binding affinity as measured by KD (equilibrium dissociation constant) for human TREM2 and for human Gpnmb of about 10⁻⁸, 10⁻⁹, 10⁻¹⁰, 10⁻¹¹ M or lower (lower values indicating higher binding affinity). In certain embodiments, the antigen binding proteins of the invention specifically bind to human TREM2 and for human Gpnmb with a KD from about 1 pM to about 100 nM as measured by bio-layer interferometry at 25° C. For instance, in some embodiments, the antigen binding proteins of the invention specifically bind to human TREM2 and for human Gpnmb with a KD less than 100 nM as measured by bio-layer interferometry at 25° C. In other embodiments, the antigen binding proteins of the invention specifically bind to human TREM2 and for human Gpnmb with a KD less than 50 nM as measured by bio-layer interferometry at 25° C. In yet other embodiments, the antigen binding proteins of the invention specifically bind to human TREM2 and for human Gpnmb with a KD less than 25 nM as measured by bio-layer interferometry at 25° C. In one particular embodiment, the antigen binding proteins of the invention specifically bind to human TREM2 and for human Gpnmb with a KD less than 10 nM as measured by bio-layer interferometry at 25° C. In another particular embodiment, the antigen binding proteins of the invention specifically bind to human TREM2 and for human Gpnmb with a KD less than 5 nM as measured by bio-layer interferometry at 25° C. In another particular embodiment, the antigen binding proteins of the invention specifically bind to human TREM2 and for human Gpnmb with a KD less than 1 nM as measured by bio-layer interferometry at 25° C.

It will be appreciated that the antibodies of the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the antibody/ies of the present invention (e.g., the antibody) which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, neurosurgical strategies (e.g., intracerebral injection, intrastriatal infusion or intracerebroventricular infusion, intra spinal cord, epidural), transmucosal, intestinal or parenteral delivery, including intramuscular, subcutaneous and intramedullary injections as well as intrathecal, direct intraventricular, intracardiac, intravenous, intraperitoneal, intranasal, or intraocular injections.

Alternately, one may administer the pharmaceutical composition in a local rather than a systemic manner, for example, via injection of the pharmaceutical composition directly into a tissue region of a patient (e.g. adipose tissue).

According to a preferred embodiment, the antibody/ies are not administered into the brain of the subject.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose (e.g. reduction of number or size of adipocytes, or decrease in the amount of visceral fat).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (Scc e.g., Fingl, et al., 1975, in “The Pharmacological Basis of Therapeutics”, Ch. 1p.1).

Dosage amount and interval may be adjusted individually to provide tissue levels of the active ingredient that are sufficient to decrease the number or size of adipocytes or decrease visceral fat (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The present inventors contemplate administering to the subject (in combination with the above described antibody/ies that target the TREM2/Gpnmb expressing cells) additional chemotherapeutic agents. Such agents may work synergistically with the above described antibody/ies for the treatment of cancer.

Treatment can be combined with any anti-cancer treatment known in the art, including, but not limited to, chemotherapeutic agents, radiotherapeutic agents, hormonal therapy, immune modulators, engineered immune cell therapy (e.g., CAR-T) and other treatment regimens (e.g., surgery, cell transplantation e.g. hematopoietic stem cell transplantation) which are well known in the art.

The chemotherapeutic agent of the present invention can be, but not limited to, cytarabine (cytosine arabinoside, Ara-C, Cytosar-U), asprin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as ⁵-fluorouracil, fluorodeoxyuridine, gemcitabine, cytosine arabinoside (AraC, cytarabine), 5-azacytidine, 2,2· difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; cpipodophylotoxins such as ctoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurca, methylhydrazine derivatives including Nmethylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

In some embodiments the chemotherapeutic agent of the present invention is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, procarbazine, pentostatin, mechlorethamine, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, Cytosar-U), melphalan, busulfan (Myleran®), thiotepa, bleomycin, platinum (cisplatin), cyclophosphamide, Cytoxan®)., daunorubicin, doxorubicin, idarubicin, mitoxantrone, 5-azacytidine, cladribine, fludarabine, hydroxyurea, 6-mercaptopurine, methotrexate, 6-thioguanine, or any combination thereof.

According to a specific embodiment, the treatment is combined with an immune checkpoint inhibitor, such as described below.

As used herein “immune checkpoint inhibition” refers to cancer immunotherapy. The therapy targets immune checkpoints, key regulators of the immune system that stimulate or inhibit its actions, which tumors can use to protect themselves from attacks by the immune system. Checkpoint therapy can block inhibitory checkpoints, activate stimulatory functions, thereby restoring immune system function. Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274).

Examples of immune checkpoint inhibitors include, but are not limited to, of cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD244 (2B4), CD160, GARP, OX40, CD137 (4-1BB), CD25, VISTA, BTLA, TNFR25, CD57, CCR2, CCRS, CCR6, CD39, CD73, CD4, CD18, CD49b, CD1d, CDS, CD21, TIMI, CD19, CD20, CD23, CD24, CD38, CD93, IgM, B220 (CD45R), CD317, CD11b, Ly6G, ICAM-1, FAP, PDGFR, Podoplanin, and TIGIT.

Examples of clinically approved immune checkpoint inhibitors include, but are not limited to, Ipilimumab, (anti CTLA-4), Nivolimumab (anti PD-1) and Pembrolizumab (anti PD 1).

According to another embodiment, the treatment is combined with a Brutons tyrosine kinasc (Btk) inhibitor (e.g. ibrutinib, acalabrutinib or Spebrutinib).

The present inventors also contemplate selecting a treatment type based on the presence of myeloid cells which express both TREM2 and Gpnmb.

Thus, according to still another aspect of the present invention there is provided a method of treating cancer in a subject comprising:
(a) analyzing in a sample of the subject for the presence of myeloid cells which express both TREM2 and Gpnmb; and
(b) when there is an amount of said myeloid cells above a predetermined amount, treating the subject with a therapeutically effective amount of antibody/ies that targets TREM2 and/or Gpnmb as described herein; or when there is an amount of said cells below a predetermined amount treating the subject with a therapeutically effective amount of a chemotherapeutic agent other than said antibody/ies that targets TREM2 and/or Gpnmb.

Methods of determining gene expression profiles can be performed at the RNA or protein level.

Below is a more detailed description of methods that can be used to analyze expression of a plurality of genes on the single cell level.

Methods of Analyzing and/or Quantifying RNA

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo GJ, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, CA).

### Single Cell Transcriptome Analysis

This method relies on sequencing the transcriptome of a single cell. In one embodiment a high-throughput method is used, where the RNAs from different cells are tagged individually, allowing a single library to be created while retaining the cell identity of each read. The method can be carried out a number of ways—see for example US Patent Application No. 20100203597 and US Patent Application No. 20180100201, the contents of which are incorporated herein by reference.

One particular method for carrying out single cell transcriptome analysis is summarized below.

Cells are typically aliquoted into wells such that only one cell is present per well. Cells are treated with an agent that disrupts the cell and nuclear membrane making the RNA of the cell accessible to sequencing reactions.

According to one embodiment, the RNA is amplified using the following in vitro transcription amplification protocol:

(Step 1) contacting the RNA of a single cell with an oligonucleotide comprising a polydT sequence at its terminal 3′ end, a T7 RNA polymerase promoter sequence at its terminal 5′ end and a barcode sequence positioned between the polydT sequence and the RNA polymerase promoter sequence under conditions that allow synthesis of a single stranded DNA molecule from the RNA, wherein the barcode sequence comprises a cell barcode and a molecular identifier;

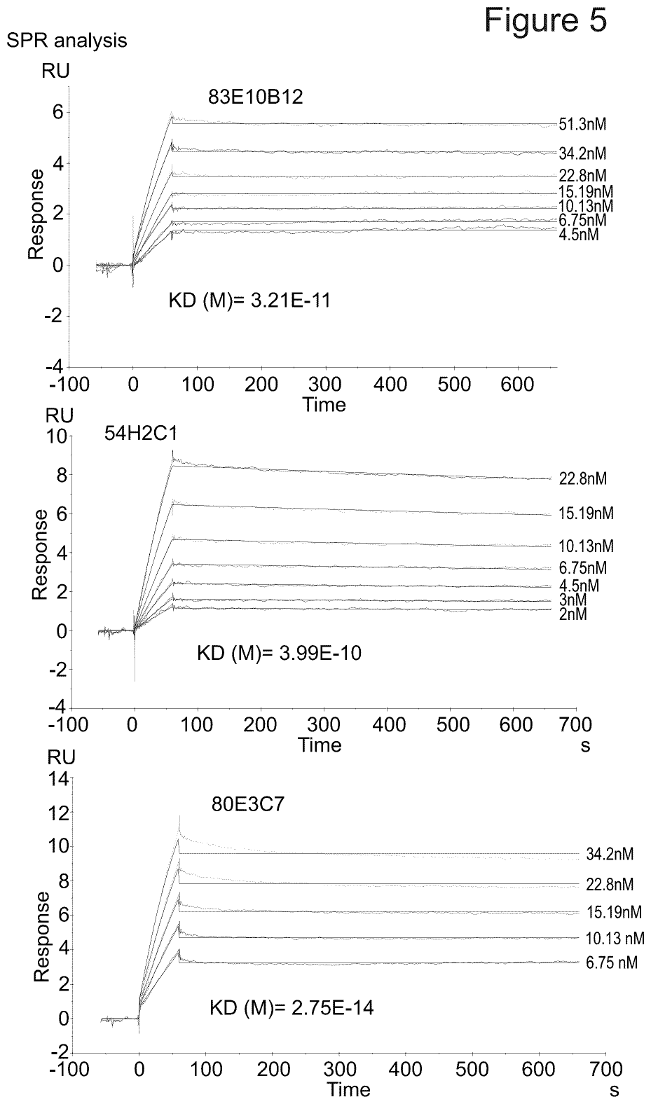

The polydT oligonucleotide of this embodiment may optionally comprise an adapter sequence required for sequencing—see for example FIG. 5.

RNA polymerase promoter sequences are known in the art and include for example T7 RNA polymerase promoter sequence e.g.

(SEQ ID NO: 3)
SCGATTGAGGCCGGTAATACGACTCACTATAGGGGC.

Preferably the polydT sequence comprises at least 5 nucleotides. According to another embodiment the polydT sequence is between about 5 to 50 nucleotides, more preferably between about 5-25 nucleotides, and even more preferably between about 12 to 14 nucleotides.

The barcode sequence is useful during multiplex reactions when a number of samples are pooled in a single reaction. The barcode sequence may be used to identify a particular molecule, sample or library. The barcode sequence is attached 5′ end of polydT sequence and 3′ of the T7 RNA polymerase sequence. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides.

In one embodiment, the barcode sequence is used to identify a cell type, or a cell source (e.g. a patient).

Molecular identifiers are useful to correct for amplification bias, which reduces quantitative accuracy of the method. The molecular identifier comprises between 4-20 bases. The molecular identifier is of a length such that each RNA molecule of the sample is catalogued (labeled) with a molecular identifier having a unique sequence.

Following annealing of a primer (e.g. polydT primer) to the RNA sample, an RNA-DNA hybrid may be synthesized by reverse transcription using an RNA-dependent DNA polymerase. Suitable RNA-dependent DNA polymerases for use in the methods and compositions of the invention include reverse transcriptases (RTs). RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules. However, in some instances, it is preferable to employ a RT which lacks or has substantially reduced RNase H activity.

RTs devoid of RNase H activity are known in the art, including those comprising a mutation of the wild type reverse transcriptase where the mutation eliminates the RNase H activity. Examples of RTs having reduced RNase H activity are described in US20100203597. In these cases, the addition of an RNase H from other sources, such as that isolated from E. coli, can be employed for the formation of the single stranded cDNA. Combinations of RTs are also contemplated, including combinations of different non-mutant RTs, combinations of different mutant RTs, and combinations of one or more non-mutant RT with one or more mutant RT.

Examples of suitable enzymes include, but are not limited to AffinityScript from Agilent or Superscript III from Invitrogen. Preferably the reverse transcriptase is devoid of terminal Deoxynucleotidyl Transferase (TdT) activity.

Additional components required in a reverse transcription reaction include dNTPS (dATP, dCTP, dGTP and dTTP) and optionally a reducing agent such as Dithiothreitol (DTT) and MnCl2.

The polydT oligonucleotide may be attached to a solid support (e.g. beads) so that the cDNA which is synthesized may be purified.

Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° ° C., usually from about 55° C. to about 70° C., and more usually from about 60° ° C. to about 68° C. Annealing conditions are generally maintained for a period of time ranging from about 15 seconds to about 30 minutes, usually from about 30 seconds to about 5 minutes.

(Step 2): Once cDNA is generated, the cDNA may be pooled from cDNA generated from other single cells (using the same method as described herein above).

The sample may optionally be treated with an enzyme to remove excess primers, such as exonuclease I. Other options of purifying the single stranded DNA are also contemplated including for example the use of paramagnetic microparticles. This may be carried out following or prior to sample pooling.

(Step 3): Second strand synthesis.

Second strand synthesis of cDNA may be effected by incubating the sample in the presence of nucleotide triphosphates and a DNA polymerase. Commercial kits are available for this step which include additional enzymes such as RNAse H (to remove the RNA strand) and buffers. This reaction may optionally be performed in the presence of a DNA ligase. Following second strand synthesis, the product may be purified using methods known in the art including for example the use of paramagnetic microparticles.

(Step 4): Following synthesis of the second strand of the cDNA, RNA may be synthesized by incubating with a corresponding RNA polymerase. Commercially available kits may be used such as the T7 High Yield RNA polymerase IVT kit (New England Biolabs).

(Step 5): Prior to fragmentation of the amplified RNA, the DNA may be removed using a DNAse enzyme. The RNA may be purified as well prior to fragmentation. Fragmentation of the RNA may be carried out as known in the art. Fragmentation kits are commercially available such as the Ambion fragmentation kit.

(Step 6): The amplified and fragmented RNA is now labeled on its 3′ end. For this a ligase reaction is performed which essentially ligates single stranded DNA (ssDNA) to the RNA. Other methods of labeling the amplified and fragmented RNA are described in US Application No. 20170137806, the contents of which are incorporated herein by reference. The single stranded DNA has a free phosphate at its 5′end and optionally a blocking moiety at its 3′end in order to prevent head to tail ligation. Examples of blocking moieties include C3 spacer or a biotin moiety. Typically, the ssDNA is between 10⁻⁵⁰ nucleotides in length and more preferably between 15 and 25 nucleotides.

(Step 7): Reverse transcription is then performed using a primer that is complementary to the primer used in the preceding step. The library may then be completed and amplified through a nested PCR reaction as illustrated in FIG. 5.

### (Step 8): Amplification

Once the adapter polynucleotide of the present invention is ligated to the single stranded DNA (i.e. further to extension of the single stranded DNA), amplification reactions may be performed.

### (Step 9): Sequencing

Methods for sequence determination are generally known to the person skilled in the art. Preferred sequencing methods are next generation sequencing methods or parallel high throughput sequencing methods e.g. Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLID technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

It will be appreciated that methods which rely on microfluidics can also be used to carry out single cell transcriptome analysis.

Thus, a combination of molecular barcoding and emulsion-based microfluidics to isolate, lyse, barcode, and prepare nucleic acids from individual cells in high-throughput may be used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode about 10,000-100,000 cells. In this regard there can be a single-cell sequencing library which may comprise: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells: and preparing and sequencing a single composite RNA-Seq library, as described herein above. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2, all the contents and disclosure of each of which are herein incorporated by reference in their entirety.

Methods of Detecting Expression and/or Activity of Proteins

Expression and/or activity level of proteins expressed in the cells of the cultures of some embodiments of the invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to a specific embodiment, the gene expression is determined by transcriptome analysis.

According to a specific embodiment, the gene expression is determined by a single cell transcriptome analysis as described above.

Thus, once a particular level of cells is observed e.g., more than 5%, more than 10% of myeloid cells of a sample derived from the, the subject can be considered as a candidate for a therapy that targets these cells. If an insufficient number of myeloid cells of this signature is observed, the subject is not considered as a candidate for this therapy.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., cd. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., cd. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., cd. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

### Anti Human TREM2 Monoclonal Antibodies Production

Five BalB/C and 5 SJL mice were immunized with recombinant protein consisting of the extracellular domain of human TREM2 fused to the His tag (SEQ ID NO: 4 MEPLRLLILLFVTELSGAHNTTVFQGVAGQSLQVSCPYDSMKHWGRRKAWCRQLGEK GPCQRVVSTHNLWLLSFLRRWNGSTAITDDTLGGTLTITLRNLQPHDAGLYQCQSLHGS EADTLRKVLVEVLADPLDHRDAGDLWFPGESESFEDAHVEHSISRSLLEGEIPFPPTSHH HHHH). Mice's spleen were harvested and fused with Sp2/0 myeloma cells. Ab-producing clones were selected by ELISA and screened against 293HEK cells that had been stably transfected with hTREM2.

### Direct ELISA

Ninety six-Well ELISA microplate was coated with 0.5 μg/mL, 100 μl/well His tagged hTREM2 diluted in PBS pH 7.4, and incubated at 4° C. overnight. The plate was rinsed three times with 0.05% tween20 in PBS, blocked with 1% BSA in PBS at room temperature (RT) for 1 hr, and rinsed again. The plates was incubated with the anti hTREM2 antibodies (100 μl/well) for 2 hrs at indicated concentration, at RT. Plate was rinsed and incubated with Peroxidase-AffiniPure Goat anti Mouse IgG, Fcg fragment specific (min X Hu, Bov, Hrs, Sr Prot; Jackson ImmunoResearch) for 20 min at RT. The plate was rinsed and incubated with TMB Reagent (TM4500, Scytek) for 20 min at RT, followed by the addition of Stop Solution 2N Sulfuric Acid (DY994, R&D). OD was measured using an ELISA plate reader at dual wavelengths (450 nm and 570 nm)

### Sup Elisa

WT 293HEK cells or hTREM2 overexpressing 293HEK cells were cultured for 24 hrs (3 million cells in 10 cm plate). Supernatant was collected, centrifuged for 5 min at 900 g and filtered (0.45 μm). Ninety six-well ELISA microplate was coated with 0.2 μg/well of anti human TREM2 Antibody (AF1828, R&D) diluted in PBS, and incubated at 4° C. overnight. The plate was rinsed three times with 0.05% tween20 in PBS, blocked with 1% BSA in PBS at room temperature (RT) for 1 hr, and rinsed again. The plates was incubated with the cell culture supernatant (100 μl/well) for 2 hrs at RT for and rinsed again. The plate was incubated with 0.1 μg biotinylayed anti hTREM2 antibodies for 2 hrs at RT. After incubation, the plate was rinsed and incubated with streptavidin-HRP (DY998, R&D) for 20 min at RT. The plate was rinsed and incubated with TMB Reagent (TM4500, Scytek) for 20 min at RT, followed by the addition of Stop Solution 2N Sulfuric Acid (DY994, R&D). OD was measured using an ELISA plate reader at dual wavelengths (450 nm and 570 nm)

### Cell-based ELISA

WT or hTREM2 overexpressing 293HEK cells were seeded on poly L lysine-coated 96 tissue culture plates and cultured for 48 hrs in the presence of the MMP inhibitor GM-6001 (25 uM. Enzo). Cells were washed three times with PBS and blocked (5% FBS, 1% BSA in PBS) for 2 hrs at RT. Cells were washed with 1% BSA in PBS and were incubated with 0.1 ug (or indicated amount) obiotinylayed anti hTREM2 antibodies for 4 hrs at RT. After incubation, cells were incubated with streptavidin-HRP (DY998, R&D) for 20 min at RT. The cells were washed and incubated with TMB Reagent (TM4500, Scytek) for 30 min at RT, followed by the addition of Stop Solution 2N Sulfuric Acid (DY994, R&D). OD was measured using an ELISA plate reader at dual wavelengths (450 nm and 570 nm). Biotinylation of Antibodies was done using EZ-Link™ Sulfo-NHS-LC-Biotinylation kit (Thermo Fisher Scientific) according to manufacturer's instructions.

Western blot (SDS PAGE) for TREM2 detection

Cells were washed with ice-cold PBS and resuspended in cold hypotonic lysis buffer (0.01 M Tris, pH 7; 1 mM EDTA; 1 mM EGTA) supplemented with protease inhibitor cocktail (complete™, Roche) and incubated on ice for 30 min. Cells were snap frozen in liquid nitrogen, thawed and centrifuged at 16,000 g for 45 min at 4° ° C. Pellet was resuspended in STE lysis buffer (150 mM NaCl, 50 mM Tris-HCl, pH 7.6, 2 mM EDTA, 1% Triton X-100), incubated on ice for 20 min and centrifuged at 16,000 g for 30 min at 4° C. Supernatant was collected and protein concentration was measured using the BCA protein assay. Protein (50 μg) separated on 12% Bis-Tris gels and transferred onto nitrocellulose membranes (Thermo Fisher Scientific). The membranes were blocked at room temperature with 3% BSA diluted in TBS-Tween for 1 h. Membrane was incubated at 4° C. overnight with anti hTREM2 antibodies (1 μg/ml). As secondary antibody, Peroxidase-AffiniPure Goat Anti-Mouse IgG was used (Jackson ImmunoResearch; 115-035-071). The bounded antibodies were visualized using SignalFire Elite ECL Reagent (Cell Signaling Technology).

### Bone Marrow Derived Macrophages (BMDM) Differentiation

Mouse bone marrow cells of TREM2 knockout (KO) and hTREM2 transgenic (hTREM2) mice were cultured 7 days in the presence of 30 ng/ml hM-CSF cytokine (Peprotech, 300-25) to generate bone marrow derived macrophage cells (BMDM).

### Flow Cytometry Analysis

TREM2 KO and hTREM2 BMDM were washed with MACS buffer (PBS pH 7.2, 0.5% BSA and 2 mM EDTA) and stained with biotin conjugated anti hTREM2 antibodies (10 μg/mL) followed by Washing with MACS buffer and APC-streptavidin (Biolegend, 405207) incubation, then analyzed by flow cytometer (LSRII, BD).

### Surface Plasmon Resonance (SPR) Analysis

Affinity measurement was obtained by SPR, carried out on BIAcore T200 instrument, with Series S Sensor Chip CM5 (Cytiva). hTREM2-His protein was capture to chip, and anti hTREM2 antibodies as the analyte. Sensograms were fit using steady-state affinity binding to provide equilibrium dissociation constant (KD) values.

### Immunofluorescence Analysis

TREM2 KO and hTREM2 BMDM were seeded on poly L lysine-coated coverslips and cultured for 24 hrs. Cells were Washed twice with PBS, fixed with cold methanol, washed and blocked with 5% FBS in PBS for 1 hr. Cells were incubated at 4° C. overnight with anti hTREM2 antibodies (2 μg/ml). Cells were washed with PBS following by secondary antibody staining (Alexa Fluor 647-AffiniPure F(ab′)2 Fragment Donkey Anti-Mouse IgG (H+L); Jackson ImmunoResearch; 715-606) and DAPI.

### Bone Marrow Derived Macrophages (BMDM) Differentiation Perturbation Assay

Mouse bone marrow cells were differentiated into macrophage as indicated while anti hTREM2 antibodies was added to culture medium (10 μg/mL) at day 2 and 5 of culturing.

ELISA Analysis of Biotin Conjugated hTREM2 Antibody Penetration

MCA-205 cells were washed and resuspended in PBS and injected subcutaneously (0.5 million cells/mouse in 100 μl PBS). Mice were previously shaved on the flank, and injected subcutaneous (s.c). Mice were treated intraperitoneally (i.p.) with biotin conjugated anti-hTREM2 antibody (70 μg/mouse) at day 9, and scarified 24 hours afterward. For ELISA analysis indicted organs were harvested and whole protein was extracted by homogenization in RIPA lysis buffer supplemented with complete™ Protease Inhibitor Cocktail. Sample was left on ice for 20 minutes, and centrifuged at 13,000×g for 20 min at 4° C. Supernatants were collected and protein concentration was determined using BCA method. 100 μg protein were loaded on hTREM2 protein pre-coated and blocked (1% BSA in PBS) following by washing 5 times and HRP-streptavidin binding. The plate was rinsed and incubated with TMB Reagent (TM4500, Scytek) for 20 min at RT, followed by the addition of Stop Solution 2N Sulfuric Acid (DY994, R&D). OD was measured using an ELISA plate reader at dual wavelengths (450 nm and 570 nm)

### Anti-Human GPNMB Monoclonal Antibodies Production

5 C57BL/6J mice were immunized with recombinant protein consisting of the extracellular domain of human GPNMB fused to the His tag (SEQ ID NO: 5 MECLYYFLGFLLLAARLPLDAAKRFHDVLGNERPSAYMREHNQLNGWSSDENDWNEK LYPVWKRGDMRWKNSWKGGRVQAVLTSDSPALVGSNITFAVNLIFPRCQKEDANGNI VYEKNCRNEAGLSADPYVYNWTAWSEDSDGENGTGQSHHNVFPDGKPFPHHPGWRR WNFIYVFHTLGQYFQKLGRCSVRVSVNTANVTLGPQLMEVTVYRRHGRAYVPIAQVK DVYVVTDQIPVFVTMFQKNDRNSSDETFLKDLPIMFDVLIHDPSHFLNYSTINYKWSFGD NTGLFVSTNHTVNHTYVLNGTFSLNLTVKAAAPGPCPPPPPPPRPSKPTPSLATTLKSYDS NTPGPAGDNPLELSRIPDENCQINRYGHFQATITIVEGILEVNIIQMTDVLMPVPWPESSLI DFVVTCQGSIPTEVCTIISDPTCEITQNTVCSPVDVDEMCLLTVRRTFNGSGTYCVNLTLG DDTSLALTSTLISVPDRDPASPLRMANHHHHHH). Mice's′ spleen were harvested and single B cells were analyzed for GPNMB binding by flow cytometry, and sorted for BCR sequencing. Productive BCR were cloned into OG527 (IgG) and OG528 (Igk) for antibodies production.

### Anti-Human GPNMB Monoclonal Antibodies Binding Screening

Anti-human GPNMB antibodies will be screened for binding by direct ELISA and flow cytometry with hGPNMB expressing 293HEK as well as human peripheral blood derived macrophage.

### Anti-Human GPNMB Monoclonal Antibodies Function Screening

Human white blood cells were extracted from peripheral blood by Ficoll separation. CD4 T cells were isolated using CD4 microbeads (Miltenyi Biotec, 130-045-101). CD4 cells were incubated in pre-coated anti CD3, anti CD28 and anti CD2 antibodies (Miltenyi Biotec, 130-091-441) with recombinant hGPNMB protein, for 1-3 days. Number of replications were measured by CFSE staining using flow cytometry. IFNg secretion was measured by ELISA (Biolegend, BLG-430104). CD4 T cell activation suppression ability of anti hGPNMB antibodies test will execute by adding 10 μg/mL antibody during T cell incubation.

Human Monocyte Derived Macrophage (hMDM) and Human Monocyte Derived Dendritic Cells (hMDC) Differentiation

CD14+ cells were isolated from human blood using CD14+isolation kit (Miltenyi Biotech #130-050201). Cells were cultured 5 days in the presence of 30 ng/ml hM-CSF cytokine (Peprotech, 300-25) following by 20 ng/mL IL4 administtration to generate M2 macrophages. To generate hMDC, cells were cultured 7 days in the presence of 30 ng/mL hGM-CSF cytokine (Peprotech, 300-03).

hMDM Stimulation

Primary hMDM were generated as described above. 10 μg/mL anti TREM2 and/or 20 ng/ml IL2 or IL15 cytokines were added to culture on day 3, 5 and 6.

### Gene Expression Analysis

For gene expression analysis cells were washed twice with PBS followed by cell lysis and RNA purification by Dynabeads mRNA DIRECT Purification Kit (Thermo Fisher, 61012) according to manufacturer instructions. cDNA was generated using SuperScript III kit (Thermo Fisher, 18080044), according to manufacturer instructions.

qPCR analysis done with Syber green (LightCycler 480 SYBR Green, Roche, 04-887-352) reagent, and the following primers (Table C):

TABLE C

| Gene | Forward primer/(SEQ ID NO:) | Reverse primer/(SEQ ID NO:) |
|---|---|---|
| CCL23 | CCGTGTTCACTCCTGGAGAGTT/501 | GCTTCAGCATTCTCACGCAAACC/ |
| | | 506 |
| S100A8 | ATGCCGTCTACAGGGATGACCT/502 | AGAATGAGGAACTCCTGGAAGTTA/ |
| | | 507 |
| S100A9 | GCACCCAGACACCCTGAACCA/503 | TGTGTCCAGGTCCTCCATGATG/ |
| | | 508 |
| IFI6 | GCTAGAGTGCAGTGGCTATT/504 | GTAATCCTACTTGGGAGGTTGAG/ |
| | | 509 |
| Ccl18 | GTTGACTATTCTGAAACCAGCCCC/ | GTCGCTGATGTATTTCTGGACCC/ |
| | 505 | 510 |

### Cytokine Secretion Analysis

Cytokine secretion analysis done on Macrophage supernatant 24 hours after M2 polarization process. Supernatant was diluted 1:20 and cytokines values measurement was done with CBA human inflammatory cytokine kit (BD551811) according to manufacturer instructions.

Isolation, Activation and Co-Culture of Human T Cells and hMDMs

Primary hMDM were generated as previously described. Following CD14+ cells selection, CD14- were frozen for the time of macrophage differentiation. 6 days later cells were thawed and CD8 T cells were selected by human CD8 microbeads (Miltenyi Bitec, 130-045-201). CD8 T cells were stained by proliferation tracking dye (65-0842-85, Thermo Fisher) and seeded on anti CD3 (10 μg,mL. OKT, BLG-317326,) and anti CD28 (2 μg/mL, BLG-302934) pre-coated plates. Differentiated and treated M2 macrophage were added to co culture in ratio of 1:3 (Macrophage: T cells). 4 days after cells were analyse for proliferation rate by flow cytometry.

### Recombinant Antibodies Binding Assays

Direct Elisa was performed as previously described. In brief, Elisa plates were coated with recombinant human TREM2 (2 μg/ml). After blocking, the plates were incubated with the recombinant antibodies at different concentrations. For the detection of the antibodies, the following secondary antibodies were used: anti-human IgG-HRP (709-035-149, Jackson ImmunoResearch), human IL2 (BLG-500302, BioLegend) followed by anti-rat IgG-HRP (ab97057, Abcam). Sup Elisa was performed as previously described. anti-TREM2 antibody AF1828 (R&D) was used as a capture antibody, followed by blocking and incubation with supernatant of hMDM. The anti-TREM2 recombinant antibodies were used as detection antibody, followed by secondary antibodies detection anti-human IgG-HRP (709-035-149, Jackson ImmunoResearch), human IL2 (BLG-500302, BioLegend) followed by anti-rat IgG-HRP (ab97057, Abcam).

### Bone Marrow Derived Macrophages (BMDM) Differentiation and Treatment

Mouse bone marrow cells from hTREM2 transgenic (hTREM2) mouse (female, 12 weeks) were extracted from femur and tibia bones, and seeded in a 96-well non-tissue culture plate (20K cells in 100 μl C10 medium) in the presence of 30 ng/ml hM-CSF cytokine (Peprotech, 300-25) to generate bone marrow derived macrophage cells (BMDM). On day 2, medium was replaced with 100 μl C10 medium supplied with 30 ng/ml hM-CSF (Control) or 30 ng/mL hM-CSF+10 μg/ml of the different antibodies (recE3C7 (recombinant anti-human TREM2), E3C7 (anti-human TREM2), recE3C7-Long-IL2, recE3C7-Short-IL2 or IgG (Isotype control)) (See Table 1). On day 5, the procedure of day 2 was repeated, with the addition of adding 20 ng/ml murine IL-4 (Peprotech, 214-14) to all of the conditions. On day 7, medium was aspirated, cells were washed once with 200 μl PBS (−/−) and 44K activated pan T cells were added to the different conditions of BMDMs in plain 100 μl C10 medium.

### Mouse T Cell Isolation and Activation

Using Miltenyi (Catalog #130-095-130) mouse Pan T cell isolation kit, T cells were isolated from WT mouse spleen (female, 12 weeks). Isolated T cells were cultured (3×10⁵ cells in 200 μl C10 medium supplied with 2 μg/ml anti-mouse CD28 (BLG-102116)) in a U shape 96 well tissue culture plate (precoated with 0.5 μg/well anti-mouse CD3 (BLG-100340)). Cells were kept in culture for 24 hours. For control, 3×10⁵ cells naive T cells were cultured in the same plate in different wells without CD3 coating and without CD28 addition to the medium. Cells were kept in culture for 24 hours.

### Co-Culture of T Cells and BMDMs

One day post T cells activation, activated and naive T cells were pipetted out of wells and transferred into 15 ml tubes separately. Cells were centrifuged 400 g for 5 min and then resuspended in C10 medium in a concentration of 44K cells/100 μl. Activated T cells were added to the BMDMs wells (44K cells in 100 μl volume) that were washed with PBS (−/−). For activated/naive T cell only controls, activated and naive T cells were seeded (44K cells/100 μl C10) in U shape 96 well tissue culture plate. Following 42 hours, supernatant was collected from all wells for IFNg release measurement.

### Mouse Interferon Gamma Secretion Measurement

Interferon gamma (IFNg) concentration was measured using ELISA MAX Deluxe Set for mouse (BGL-430804, Biolegend) and human (BLG-430115, Biolegend) IFN-γ. 96-Well ELISA microplates were coated with 0.2 μg/well of anti-mouse IFN-γ Antibody) diluted in PBS, and incubated at 4° C. overnight. The plates were rinsed three times with 0.05% tween-20 in PBS, blocked with 1% BSA in PBS at room temperature (RT) for 1 hour, and rinsed again. The coated plates were incubated with the cell culture supernatant (100 μl/well) for 2 hours at RT and rinsed again. The plates were incubated with 0.1 μg biotinylated anti mouse IFN-γ antibody for 2 hours at RT. After incubation, plates were rinsed and incubated with streptavidin-HRP for 20 min at RT. The plates were rinsed and incubated with solution F substrate for 10 min at RT, followed by the addition of Stop Solution 2N Sulfuric Acid (DY994, R&D). OD was measured using an ELISA plate reader at dual wavelengths (450 nm and 570 nm).

### Anti-Human TREM2 CAR T Cells Design and Cloning

For the design of Anti-human TREM2 CAR T cells, the sequences of the recognition domains of the anti-human TREM2 antibody #80E3C7 replaced the counterpart domains in the MSGV-1D3-28Z All ITAMs intact (Addgene #107226). A gblock which included the entire variable region sequence of the TREM2 CAR construct and a T2A-BFP was ordered and cloned into the MSGV-1D3-28Z using the restriction enzymes Ncol (NEB #R3193S) and Sall (NEB #R3138S).

### Anti-Human TREM2 CAR T Cells Production

Retroviral particles were produced by transfecting Retroviral Packaging PLAT-E cells with confluence of 70-80% grown in a 6-well or 10 cm plate. PLAT-E cells were provided with supplemented DMEM without penicillin and streptomycin 5 hours before the transfection. Transfection was performed using Lipofectamine2000 (ThermoFisher #11668027) under manufacturer instructions and included the retrovirus packaging vector Pcl-Eco (Addgene #12371) and the retrovirus CAR T expression plasmid. Media was replaced 5-13 hours post-transfection. After 48h, transfection efficiency was examined using a fluorescence microscope. Media containing retroviral particles was collected 48-72 hr post-transfection. T cells were isolated from spleens of 8-to-12-weck-old female WT mice using mouse pan T cells isolation kit II (Miltenyi Biotech #130-095-130). Cells were incubated on a 24-tissue culture plate coated with 250 ng/well of anti-CD3c (ThermoFisher #16-0031-82) with RPMI medium supplemented with 100U/ml rIL2 and 2 μg/ml anti-CD28 (Biolegend #102102). Retroviral transduction of T cells was performed by adding the retroviral particles to a 24-well non-tissue culture plate coated with Retronectin (Takara Bio #T100A) and centrifuged at 2000g for 2 hours at 32ºC. T cells were added on top of the viral soup, followed by centrifugation at 400 g for 10 mins. Cells were further expanded for 2-5 days to achieve sufficient cell numbers.

### Bone Marrow Derived Macrophages (BMDM) and Bone Marrow-Derived Dendritic Cells (BMDCs) Differentiation

Mouse bone marrow cells of WT, TREM2 knockout (KO) and hTREM2 transgenic (hTREM2) mice were cultured 7 days in the presence of 30 ng/ml hM-CSF cytokine (Peprotech, 300-25) to generate bone marrow derived macrophage cells (BMDM). To generate bone marrow derived dendritic cells (BMDC), cells were cultured 7 days in the presence of 30 ng/ml hGM-CSF cytokine (Peprotech, 300-03).

### Co-Culture of TREM2 CAR T Cells

TREM2 CAR T cells were co-cultured in 1:1 ratio with WT or hTREM2 overexpressing 293HEK, CD14+derived hM2 and hDC, and BMDM or BMDC derived from WT, TREM2 knockout (KO) or hTREM2 transgenic (hTREM2) mice. BFP expressing T cells were used as ac control for T cells response. All cells were seeded on poly L lysine-coated 96 tissue culture plates and cultured for 24 hours. Sup was taken for IFN-γ ELISA, and cells were taken for further analysis using flow-cytometry.

### Human Interferon Gamma Secretion Measurement

ELISA was done using ELISA MAX Deluxe Set Mouse IFN-γ (Biolegend #430804). 96-Well ELISA microplate was coated with 0.2 μg/well of anti-mouse IFN-γ Antibody) diluted in PBS, and incubated at 4° C. overnight. The plate was rinsed three times with 0.05% tween20 in PBS, blocked with 1% BSA in PBS at room temperature (RT) for 1 hr, and rinsed again. The plates was incubated with the cell culture supernatant (100 μl/well) for 2 hours at RT for and rinsed again. The plate was incubated with 0.1 μg biotinylayed anti mouse IFN-γ antibody for 2 hours at RT. After incubation, plate was rinsed and incubated with streptavidin-HRP for 20 min at RT. The plate was rinsed and incubated with TMB Reagent for 20 min at RT, followed by the addition of Stop Solution 2N Sulfuric Acid (DY994, R&D). OD was measured using an ELISA plate reader at dual wavelengths (450 nm and 570 nm).

### Flow Cytometry Analysis—CART Analysis

Mouse T cells were collected and washed with MACS buffer (PBS pH 7.2, 0.5% BSA and 2 mM EDTA) and stained with PE/Cy7 conjugated anti CD8a antibody, PE conjugated anti CD25 antibody. APC/Cy7 conjugated anti CD107 antibody, APC conjugated anti CD279 (PD1) antibody, followed by Washing with MACS buffer, then analyzed by flow cytometer (Symphony S6 BD).

### EXAMPLE 1

### Screening for Monoclonal Anti-TREM2 Binding

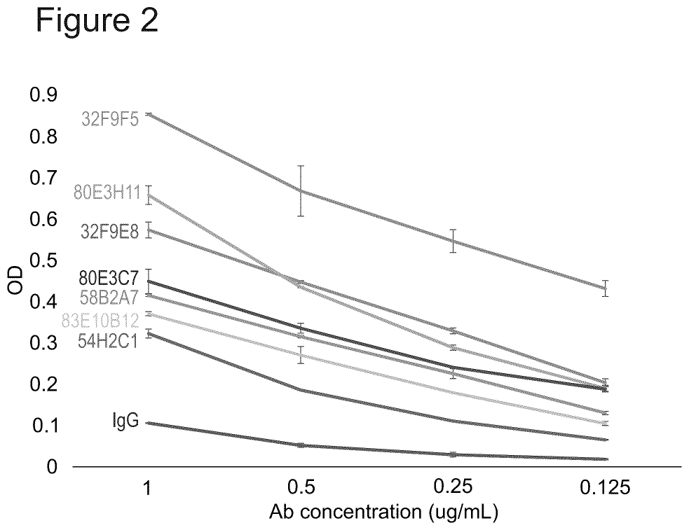

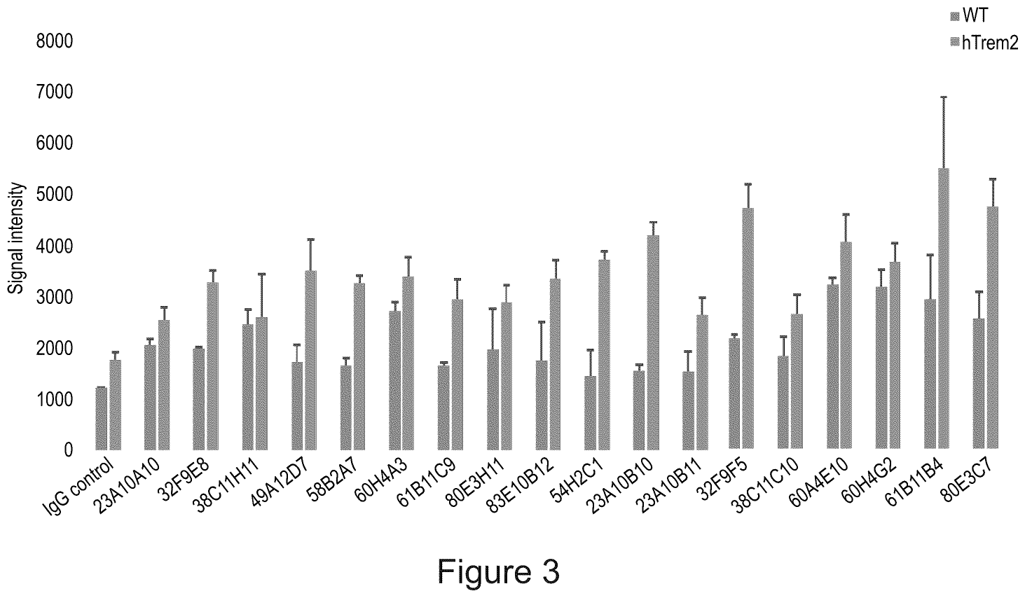

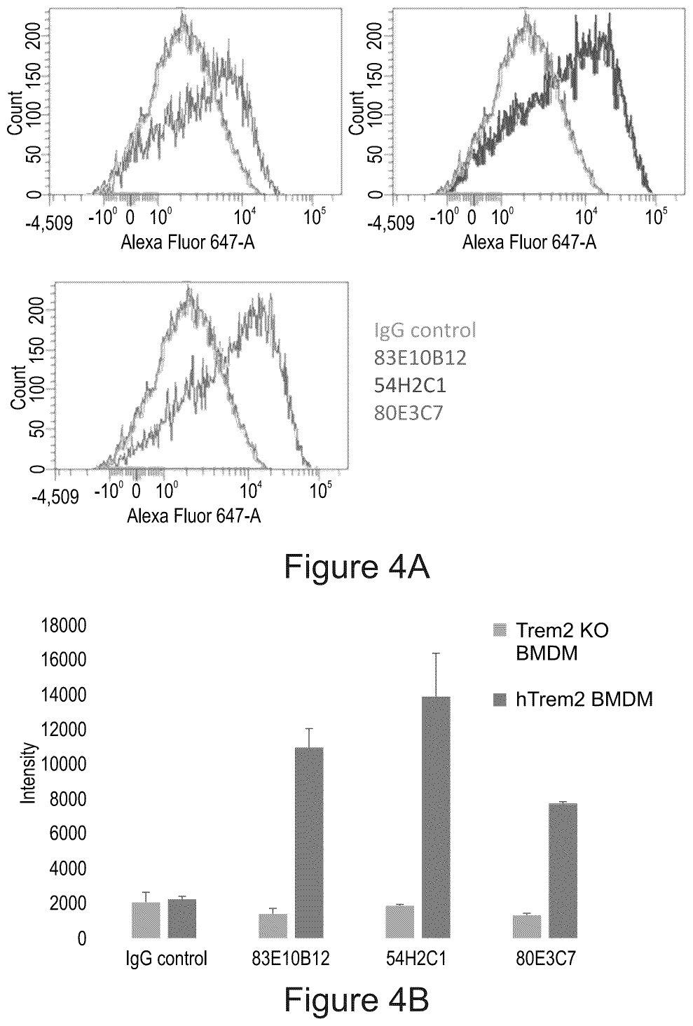

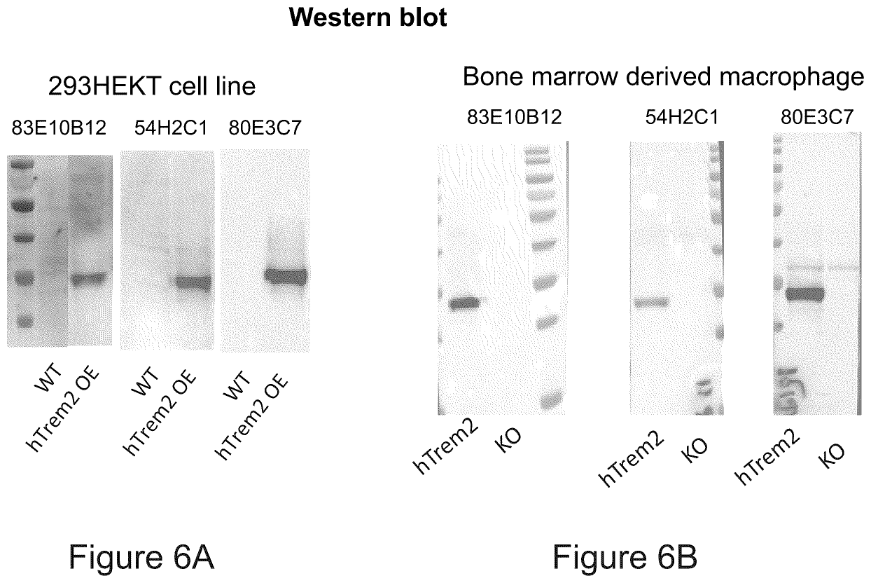

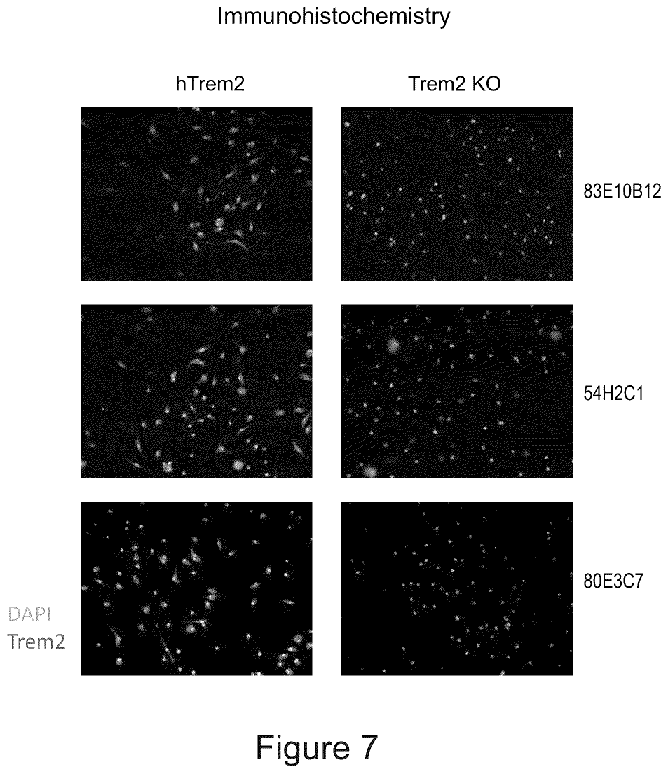

The present inventors produced and purified mouse anti human TREM2 (hTREM2) monoclonal antibodies (see above), and screened for sensitivity and specificity binding of these mAb to hTREM2. Screening was carried out first by binding measurements of TREM2 recombinant protein (Table 2, FIG. 5), as well as by comparing the mAb binding capacity to hTREM2 expressing 293HEK cells compare to WT (Table 3, FIGS. 2-3, 6A-B). It was found that bone marrow derived macrophages (BMDM) express high amount of TREM2. The present inventors extracted bone marrow cells from human TREM2 (hTREM2) transgenic mouse, and used them for bone marrow derived macrophages (BMDM) differentiation. hTREM2 BMDM were used for additional binding screening of antibodies (FIG. 4A-B, 6A-B to 7).

TABLE 1
Features summary of 18 hybridoma derived monoclonal antibodies against human TREM2 protein.

| | | | | | direct | sup ELISA | Cell based | FACS |
|---|---|---|---|---|---|---|---|---|
| | | Antibody | | | ELISA | positive/ | ELISA | positive/ |
| | | purity | EU level | | positive/ | negative | positive/negative | negative |
| Ab # | Ig type | (%) | (EU/mg) | Titer | negative | (293HEK) | (29SHEK) | BMDM |
| 23A10A10 | IgG1, Kappa | 97 | <1.87 | 2,187,000 | 32.35849057 | 9.495495495 | 1.10027473 | 1.247069 |
| 32F9E8 | IgG1, Kappa | 97 | <2.18 | 243,000 | 29.53448276 | 31.3030303 | 1.95276498 | 1.665859 |
| 38C11H11 | IgG1, Kappa | 98 | <2.36 | 243,000 | 19.3814433 | 12.7037037 | 3.0826087 | 1.05348 |
| 49A12D7 | IgG2c, Kappa | 92 | <2.01 | 2,187,000 | 34.22033898 | 8.875739645 | 1.40165062 | 2.047612 |
| 58B2A7 | IgG2b, Kappa | 97 | <1.65 | 2,187,000 | 37.18032787 | 25.05434783 | 1.74912075 | 1.982748 |
| 60H4A3 | IgG1, Kappa | 95 | <1.58 | 2,187,000 | 31.49152542 | 11.00990099 | 1.89221557 | 1.782695 |
| 61B11C9 | IgG2c, Kappa | 97 | <2.24 | 2,187,000 | 35.1147541 | 3.048275862 | 2.45517241 | 1.466334 |
| 80E3H11 | IgG1, Kappa | 97 | <1.55 | 2,187,000 | 28.30985915 | 26.33333333 | 3.0125 | 1.916691 |
| 83E10B12 | IgG2b, Kappa | 98 | <1.79 | 243,000 | 34.84745763 | 27.91358025 | 1.9125 | 2.582825 |
| 54H2C1 | IgG1, Kappa | 97 | <0.73 | 729,000 | 33.8907526 | 25.0576283 | 2.69372 | 2.720526 |
| 23A10B10 | IgG1, Kappa | 99 | <1.31 | 729,000 | 26.06153846 | 12.66666667 | 0.7238806 | 1.724578 |
| 23A10B11 | IgG1, Kappa | 95 | <1.92 | 729,000 | 29.54237288 | 11.84705882 | 0.68553459 | 2.059214 |
| 32F9F5 | IgG1, Kappa | 96 | <2.16 | 81,000 | 28.08695652 | 30.23287671 | 1.07492795 | 2.195853 |
| 38C11C10 | IgG1, Kappa | 97 | <2.83 | 81,000 | 33.31666667 | 13.51685393 | 1.41054313 | 1.459309 |
| 60A4E10 | IgG1, Kappa | 96 | <1.74 | 1,000 | 8.631578947 | 1.042654028 | 0.96914701 | 1.264031 |
| 60H4G2 | IgG1, Kappa | 94 | <1.90 | 729,000 | 27.40298507 | 10.04504505 | 1.38487395 | 1.158734 |
| 61B11B4 | IgG2c, Kappa | 96 | <2.40 | 2,187,000 | 35.74576271 | 2.702380952 | 2.34205607 | 1.883643 |
| 80E3C7 | IgG1, Kappa | 96 | <1.94 | 2,187,000 | 33.39344262 | 24.94871795 | 2.80216802 | 1.871301 |

Purity is measured by SDS-PAGE. Quantity of endotoxin (EU/mg) is measured by LAL assay.
Titer measured by direct ELISA, while the title value is the highest dilution with S/B (Signal/Blank) >= 2.1

TABLE 2
Direct ELISA analysis of 18 hybridoma derived monoclonal antibodies against human TREM2

| | sample | | | | | | | | | | | |
|---|---|---|---|---|---|---|---|---|---|---|---|---|
| Dilution | 1000 | 3000 | 9000 | 27000 | 81000 | 243000 | 729000 | 2187000 | 6561000 | 19683000 | Blank | Titer |
| 23A10A10 | 1.688 | 1.804 | 1.877 | 1.771 | 1.373 | 0.821 | 0.392 | 0.193 | 0.109 | 0.067 | 0.059 | 2,187,000 |
| 32F9E8 | 1.739 | 1.689 | 1.493 | 0.774 | 0.418 | 0.194 | 0.095 | 0.061 | 0.057 | 0.058 | 0.058 | 243,000 |
| 38C11H11 | 1.826 | 1.640 | 1.269 | 0.732 | 0.382 | 0.190 | 0.100 | 0.064 | 0.055 | 0.053 | 0.059 | 2,187,000 |
| 49A12D7 | 1.934 | 1.985 | 2.083 | 1.863 | 1.471 | 0.915 | 0.451 | 0.192 | 0.112 | 0.067 | 0.058 | 2,187,000 |
| 58B2A7 | 2.378 | 2.281 | 2.261 | 2.124 | 1.629 | 0.759 | 0.416 | 0.210 | 0.086 | 0.083 | 0.053 | 2,187,000 |
| 60H4A3 | 1.843 | 1.805 | 1.895 | 1.630 | 1.157 | 0.763 | 0.322 | 0.170 | 0.113 | 0.063 | 0.057 | 2,187,000 |
| 61B11C9 | 2.751 | 2.763 | 2.881 | 2.583 | 1.922 | 0.985 | 0.405 | 0.169 | 0.099 | 0.066 | 0.080 | 2,187,000 |
| 80E3H11 | 2.434 | 2.345 | 2.295 | 1.785 | 1.379 | 0.767 | 0.346 | 0.176 | 0.095 | 0.071 | 0.066 | 2,187,000 |
| 83E10B12 | 2.589 | 2.488 | 1.980 | 0.843 | 0.375 | 0.179 | 0.099 | 0.068 | 0.070 | 0.053 | 0.067 | 243,000 |
| 54H2C1 | 1.400 | 1.350 | 1.180 | 0.967 | 0.586 | 0.276 | 0.147 | 0.106 | 0.087 | 0.069 | 0.056 | 729,000 |
| 23A10B10 | 0.919 | 0.899 | 0.776 | 0.698 | 0.514 | 0.259 | 0.144 | 0.107 | 0.076 | 0.074 | 0.059 | 729,000 |
| 23A10B11 | 0.971 | 0.970 | 0.842 | 0.735 | 0.537 | 0.284 | 0.151 | 0.110 | 0.071 | 0.142 | 0.053 | 729,000 |
| 32F9F5 | 1.082 | 0.872 | 0.581 | 0.326 | 0.159 | 0.086 | 0.103 | 0.107 | 0.091 | 0.060 | 0.060 | 81,000 |
| 38C11C10 | 1.551 | 1.216 | 0.791 | 0.452 | 0.236 | 0.112 | 0.074 | 0.069 | 0.057 | 0.099 | 0.078 | 81,000 |
| 60A4E10 | 0.211 | 0.112 | 0.102 | 0.089 | 0.082 | 0.061 | 0.061 | 0.075 | 0.058 | 0.059 | 0.056 | 1,000 |
| 60H4G2 | 1.828 | 1.889 | 1.685 | 1.313 | 0.849 | 0.432 | 0.222 | 0.133 | 0.102 | 0.067 | 0.070 | 729,000 |
| 61B11B4 | 2.330 | 2.328 | 2.276 | 1.997 | 1.511 | 0.796 | 0.354 | 0.166 | 0.110 | 0.069 | 0.06 | 2,187,000 |
| 80E3C7 | 2.025 | 2.115 | 2.162 | 1.761 | 1.198 | 0.608 | 0.281 | 0.145 | 0.092 | 0.085 | 0.069 | 2,187,000 |

Coating antigen: His-TREM2, 0.5 μg/mL in PBS, pH 7.4, 100 μl/well.
Antibodies stock concentration: 1 mg/mL
Secondary antibody: Peroxidase-AffiniPure Goat anti Mouse IgG, Fog fragment specific (min × Hu, Bov, Hrs, Sr Prot)
The titer is the highest dilution with S/B (Signal/Blank) >= 2.1

TABLE 3
293HEK cells were stably infected with hTREM2
gene following by Puromycin selection.
A. Cells culture supernatant (Sup) was used for soluble
TREM2 protein binding capability test of 18
hybridoma derived monoclonal antibodies by
direct ELISA.
B. Cells were captured in 96 wells plate and were used
for cell based ELISA of 18 hybridoma
derived monoclonal antibodies.
OD values positive cells (hTREM2 expressing HEK293)
and negative cells (WT HEK293) are showing in the table.

| | A. Sup ELISA | | B. Cell based ELISA | |
|---|---|---|---|---|
| | Positive | Negative | Positve | Negative |
| | Cell | cell | Cell | cell |
| 23A10A10 | 1.054 | 0.111 | 0.801 | 0.728 |
| 32F9E8 | 2.066 | 0.066 | 1.695 | 0.868 |
| 38C11H11 | 1.029 | 0.081 | 2.127 | 0.69 |
| 49A12D7 | 1.5 | 0.169 | 1.019 | 0.727 |
| 58B2A7 | 2.305 | 0.092 | 1.492 | 0.853 |
| 60A4F5 | 0.61 | 0.582 | 0.599 | 0.434 |
| 60H4A3 | 1.112 | 0.101 | 1.264 | 0.668 |
| 61B11C9 | 0.442 | 0.145 | 1.78 | 0.725 |
| 80E3H11 | 1.975 | 0.075 | 1.205 | 0.4 |
| 83E10B12 | 2.261 | 0.081 | 0.918 | 0.48 |
| 54H2C1 | 1.754 | 0.07 | 1.257 | 0.467 |
| 23A10B10 | 0.988 | 0.078 | 0.776 | 1.072 |
| 23A10B11 | 1.007 | 0.085 | 0.436 | 0.636 |
| 32F9F5 | 2.207 | 0.073 | 0.746 | 0.694 |
| 38C11C10 | 1.203 | 0.089 | 0.883 | 0.626 |
| 60A4E10 | 0.66 | 0.633 | 0.534 | 0.551 |
| 60H4G2 | 1.115 | 0.111 | 0.824 | 0.595 |
| 61B11B4 | 0.454 | 0.168 | 1.253 | 0.535 |
| 80E3C7 | 1.946 | 0.078 | 1.034 | 0.369 |
| NC | 0.058 | 0.058 | 0.654 | 0.566 |

### EXAMPLE 2

### Screening for Monoclonal Anti-TREM2 Antagonists

As TREM2 loss of function showed reduced tumor growth in several mouse syngeneic tumor models, the present inventors designed an assay to effectively screen for anti-TREM2 monoclonal antibodies blocking activity. Bone marrow-derived macrophages (BMDM) are known to produce high levels of suppressive cytokines such as IL-10 and TGF-ß (Wang, BMC Immunology 2013 14:6), to determine whether TREM2 plays a role in the maturation and suppressive function of BMDM cells, the present inventors cultured bone marrow cells from the femur and tibia of TREM2 knockout (KO) and hTREM2 transgenic (hTREM2) mice 7 days in the presence of M-SCF (Methods) and characterized the temporal maturation trajectory using single cell RNA-seq. Clustering analysis and 2d projection of cells from each timepoint/genotype showed a cleared maturation trajectory with divergence between the TREM2-KO genotype and hTREM2 trajectories starting at day 5 and presenting a maximal phenotype at day 7. The WT hTREM2 BM cells showed an M2-phenotype at day 7 with high expression of Gpnmb, Lpl, Anxa1, Mmp12, Adam8, Lgals1, Lgals3, Spp1 and Lilrb4a while TREM2-KO BM genotype displayed an activated M1 phenotype, including Selenop, Ms4a4a, Fcgr2b, Ms4a7 and Lyz2 (FIGS. 8A-B). To screen for antibodies with antagonistic activity for TREM2, the present inventors cultured hTREM2 mouse bone marrow cells with M-CSF and added anti hTREM2 antibodies or IgG isotype to the medium (10 μg/mL) at day 2 and 5 of culturing. Using single cell RNA-seq, the cells were characterized at day 7 and the distribution of cells characterized between M2-phenotype (TREM2+Gpnmb+) and M1-phenotype (TREM2-) at each condition. More than 70% of TREM2-KO cells reached an M1-phenotype with less than 10% showing M2 phenotype, in contrast hTREM2 cells showed 40% M2- phenotype and only 24% M1- phenotype. Adding IgG isotype mAb to the culture did not change significantly the M1/M2 ratio and showed a similar outcome as the untreated culture, while adding anti-hTREM2 mAb 54H2C1 or 80E3C7 dramatically reduced the percentage of the M2 phenotype to 12% and 16% respectively with an increase in M1 phenotype to 69% and 63% (FIG. 8C), showing very similar maturation trajectory to TREM2-KO cells.

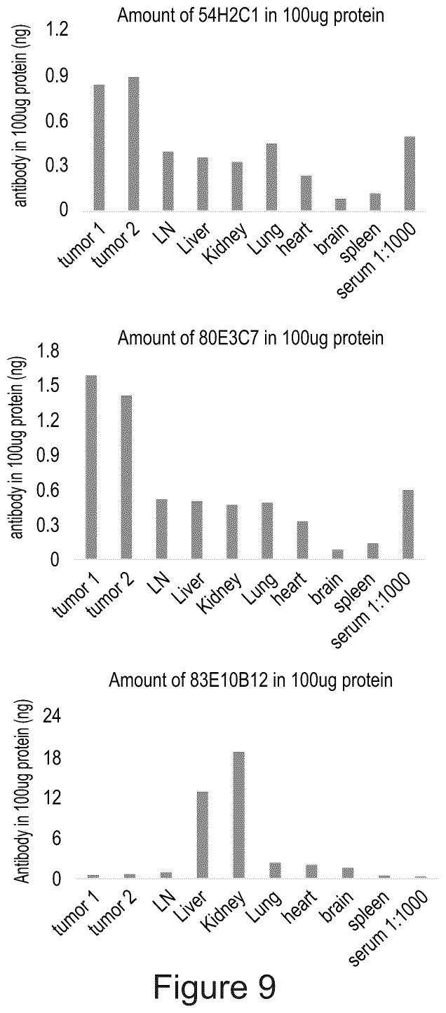

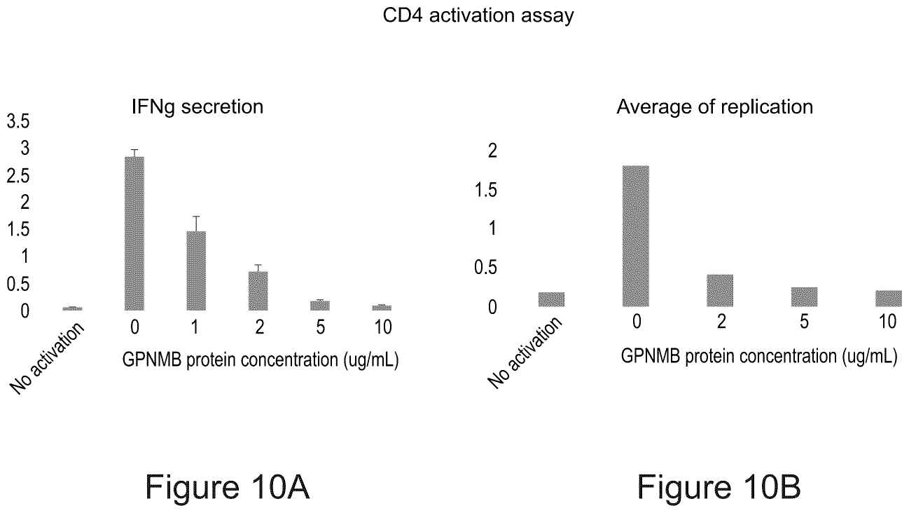

To quantify the binding specificity of anti-hTREM2 mAbs to in-vivo tumor associated macrophages and Mregs, the present inventorsperformed ELISA analysis of biotin conjugated anti-hTREM2 54H2C1, 80E3C7 and 83E10B 12 mAbs in humanized TREM2 mice harboring MCA-205 induced tumors. For mAbs 54H2C1, 80E3C7 the present inventors detected more than 2-fold enrichment of mAb concentration in the tumor TME compared to the LN, liver, kidney, lung, heart, brain and spleen. mAb 80E3C7 was mostly concentrated in the liver and kidney (see FIGS. 9-10A-B).

### EXAMPLE 3

### An Anti-TREM2 Antibody of Some Embodiments of the Invention Reprograms Human Monocytes Derived Macrophages

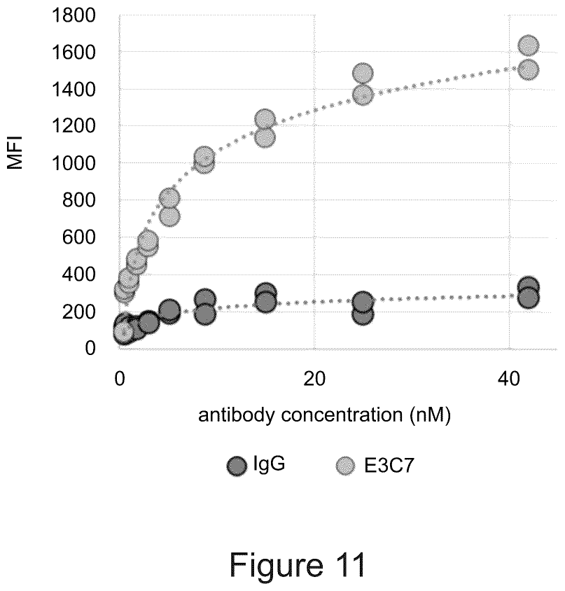

To examine the effect of antagonist TREM2 Ab E3C7 in human myeloid cells, CD14+ monocytes purified from blood of three healthy donors were differentiated into macrophages, in the presence of human M-CSF, for 5 days. The resulting monocyte-derived macrophages (MDM) were stimulated with “M2”-activating cytokines IL4 for 24h, resulting in MDM polarization to TAM-like cells. Expression of TREM2 was examined by flow cytometry analysis of TREM2 Ab antibody binding to the cell surface of those TAM-like cells (FIG. 11). The cell cultures were treated from day 3 with TREM2 Ab or with equivalent IgG control, followed by analysis of monocyte-to-TAM trajectory markers (qPCR) and protein secretion (ELISA).

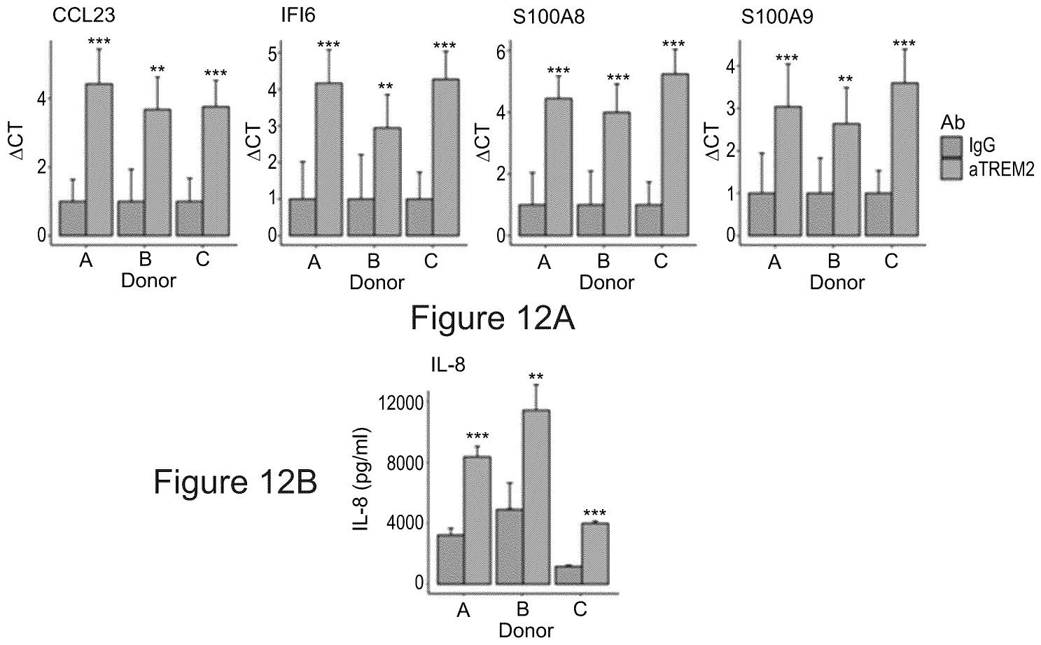

Representative genes were selected from from three key categories: (a) type I interferon activity-IFI6 (Interferon Alpha Inducible Protein 6). (b) pro-inflammatory chemokines IL-8 and CCL23. (c) S100 calcium-binding proteins A8 and A9 (S100A8 and S100A9), actively released during inflammation and play a critical role by stimulating leukocyte recruitment and inducing cytokine secretion. As shown in FIGS. 12A-B, treatment of cell cultures with E3C7 induced expression of CCL23, IFI6, S100A8 and S100A9 (A) and elevated secretion of IL8 (B) in 3 out of 3 tested donors.

The results demonstrate that antagonist TREM2 Ab interferes with MDM differentiation and reprogram immunosuppressive TAM into proinflammatory monocyte-like cells (also referred to herein as “M1-like macrophages”).

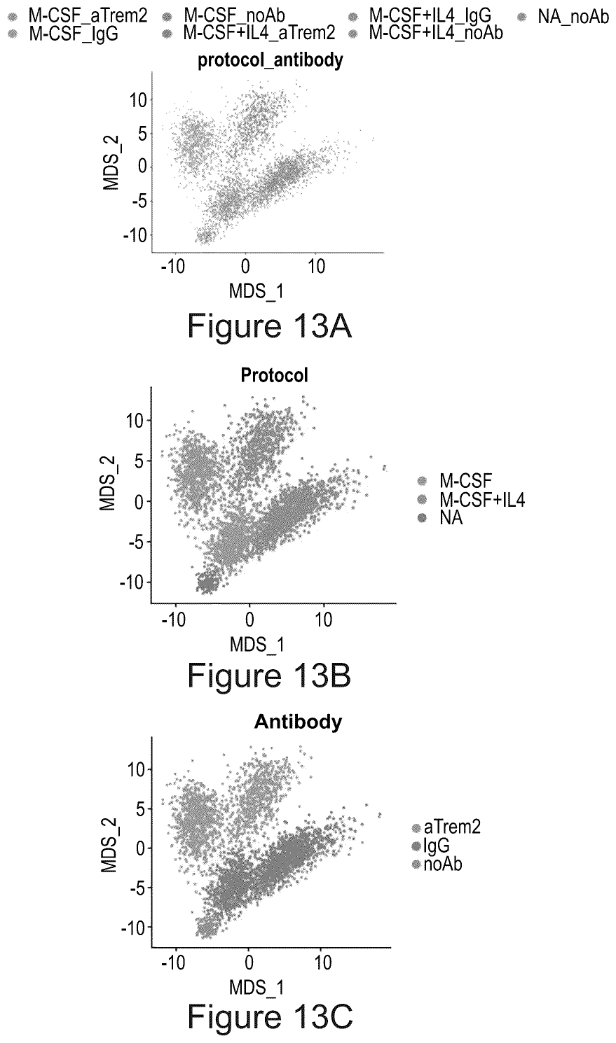

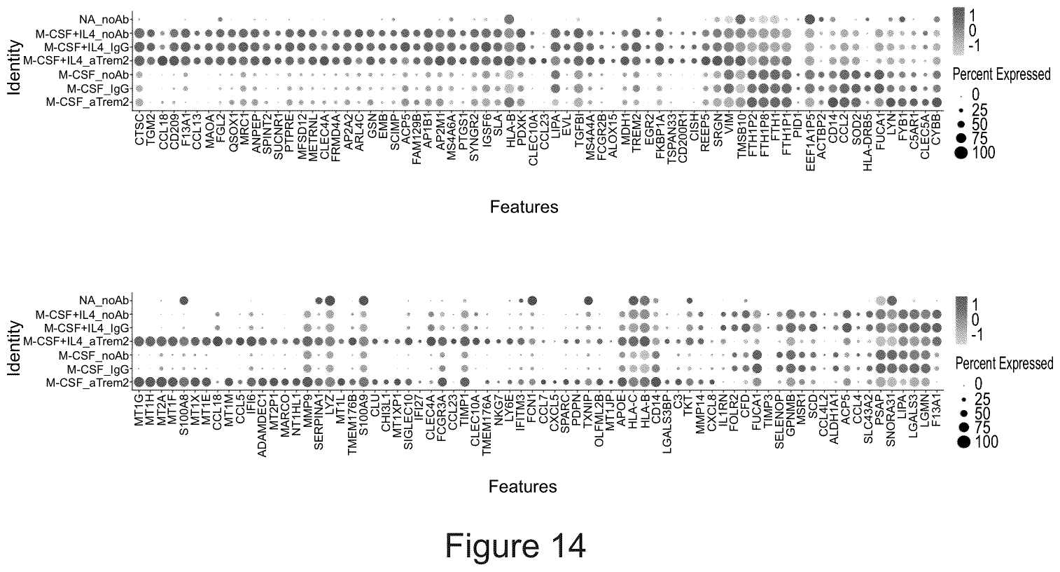

To further characterize anti-TREM2 mAb effect on monocytes derived macrophages treated with anti-TREM2 mAb MDM cultures were profiled using single cell RNA-sequencing with two differentiation conditions: M-CSF only or M-CSF+IL4 and 3 treatments: anti-TREM2E3C7, IgG control or no mAb. 2D projection of cells from 6 conditions showed a specific IL-4 effect as well as specific anti-TREM2 effect (FIGS. 13A-C). An increased expression of S100A8, CCL8, CCL23 and other proinflammatory genes was observed in cells treated with anti-TREM2 mAb, showing a strong reprogramming toward more immune active M1-like macrophages (FIG. 14).

### EXAMPLE 4

### Anti-TREM2 Antibody Attenuates Tumor Growth and Reprograms Tumor Macrophages

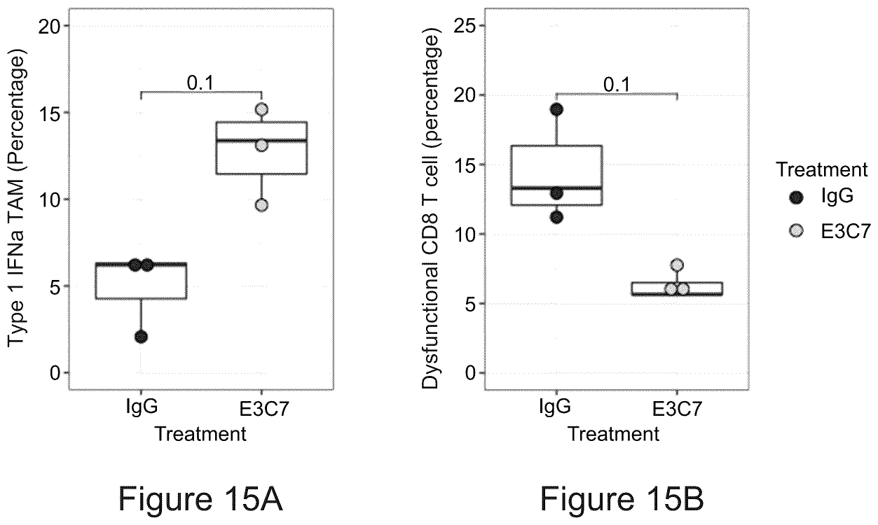

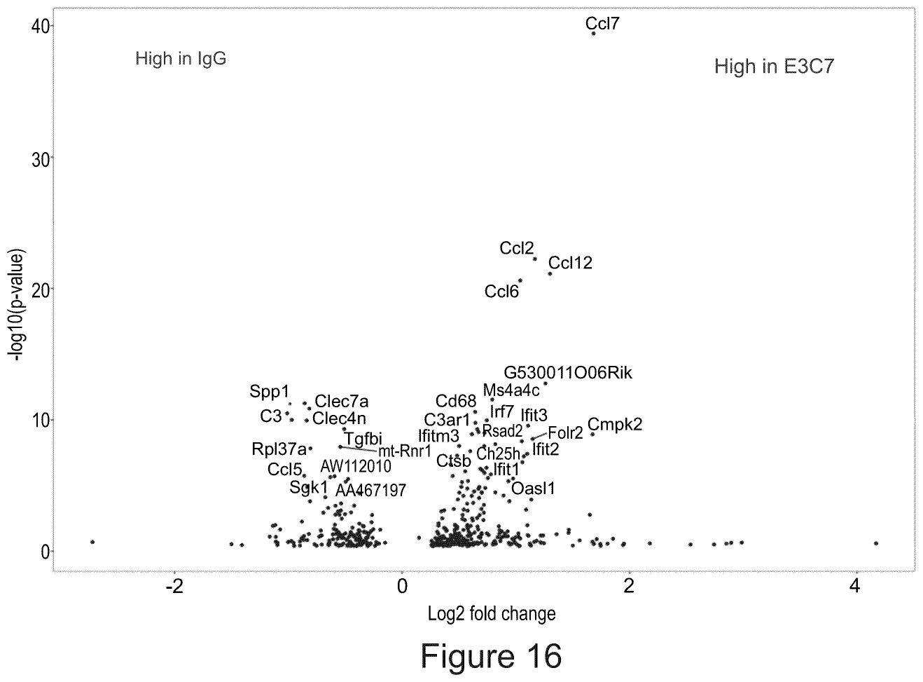

To test if TREM2 blocking reprograms macrophages in-vivo, tumor bearing mice (MCA205 syngeneic model) were treated with anti-TREM2 antibody (E3C7) or IgG control. 500K MCA-205 cells injected subcutaneously to humanized TREM2 mice and at day 6 and 9 mice were treated with with anti-TREM2 antibody or IgG control, tumors were harvested at day 10, and CD45+ positive cells were sequenced using scRNA-seq. Mice treated with anti-TREM2 (E3C7) showed increased proportion of Type-I interferon TAMs compared to control, while CD8 dysfunctional T-cells (high PDCD1 and LAG3) were reduced in E3C7 treated animals compared to control (FIGS. 15A-B). Differential gene expression analysis of tumor macrophages from E3C7 treated compared to IgG control revealed increase in Type-I IFN genes such as, Ifit1, Ifit2, Irf7 along with the Ccl7, Ccl2, Ccl6 and Ccl12 chemokines (FIG. 16).

### EXAMPLE 5

### Anti-TREM2 Antibody Cytokine Conjugation Enhances Myeloid Reprogramming and T-Cell Activation

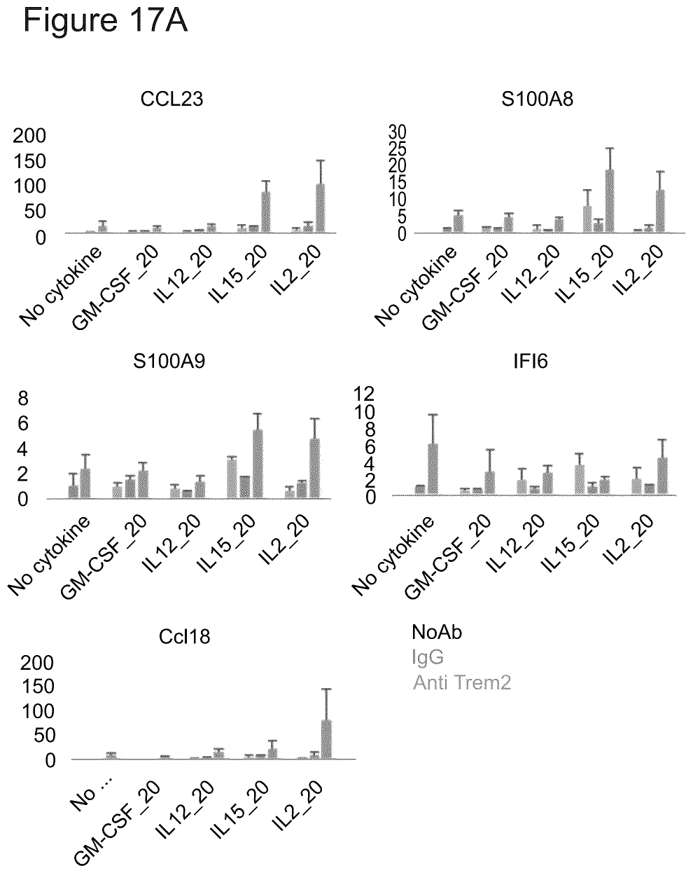

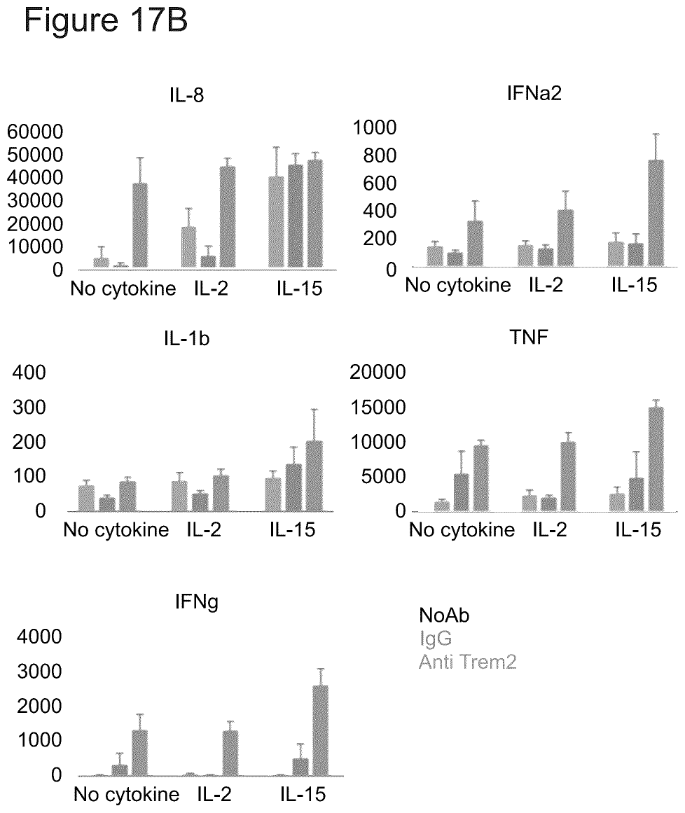

To examine the synergistic effect of anti-hTREM2 antibodies and proinflammatory cytokines, human monocyte derived macrophage (hMDM) were treated during differentiation with either anti TREM2 antibody, human IL2 (200-02-50, PeproTech), human IL15 (200-15-50, peproTech), human GM-CSF (PeproTech, AF-315-03-1000), human IL12 (R&D, 10018-IL) or a combination of anti TREM2 with one of the cytokines (FIGS. 17A-B). Gene expression analysis (FIG. 17A) shows an elevation of monocytic genes (S100A9, S100A8) and inflammation-related genes (CCL23, IFI6, CCL18) following anti TREM2 treatment, and increased after treatment with combination of anti TREM2 and IL2 or IL15. In addition, Inflammatory cytokines (FIG. 17B) secretion was elevated following anti TREM2 or cytokines administration, and increased after treatment with combination of anti TREM2 and IL2 or IL15.

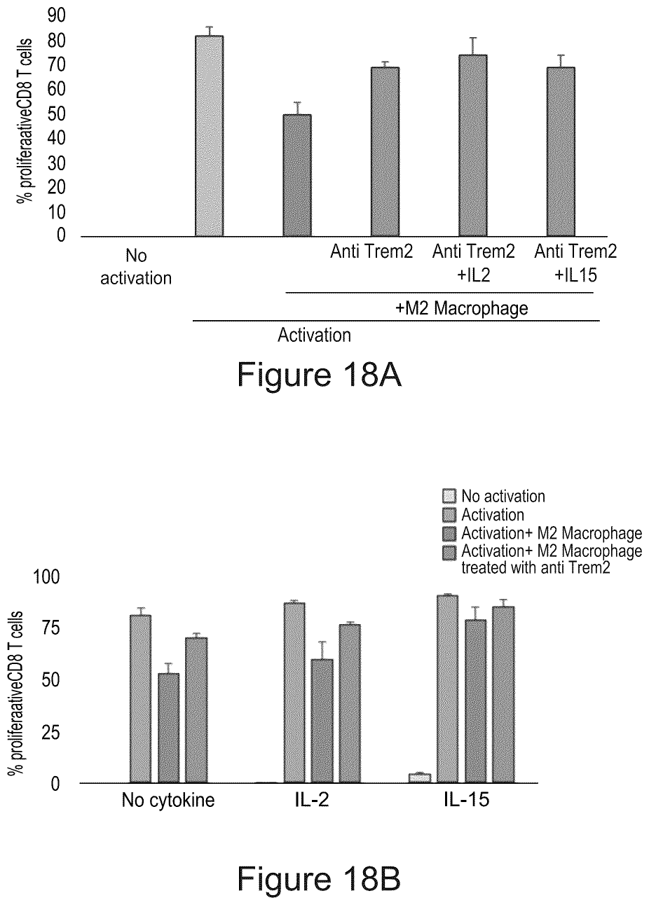

Anti TREM2 treated hMDM show reduction in their T cells suppression ability, as can be seen by the elevation of proliferative T cells percentage in FIGS. 18A-B. Moreover, this reduction intensifies following addition of IL2 or IL15 cytokines either to M2 Macrophage or to T cell-Macrophage co culture system.

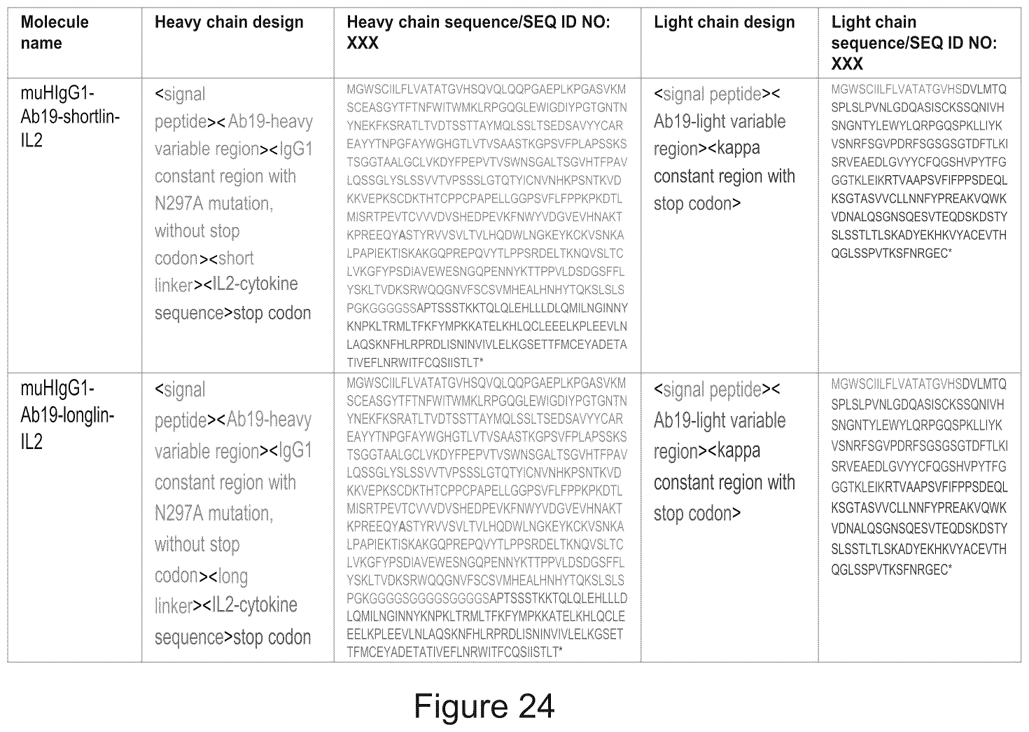

Recombinant anti-human TREM2 antibodies were generated either unconjugated and conjugated to the human IL2 cytokine (Table of FIG. 24 shows; two versions, one with short linker, and one with long linker, SEQ ID Nos: 496 and 498 or 497 and 499, respectively).

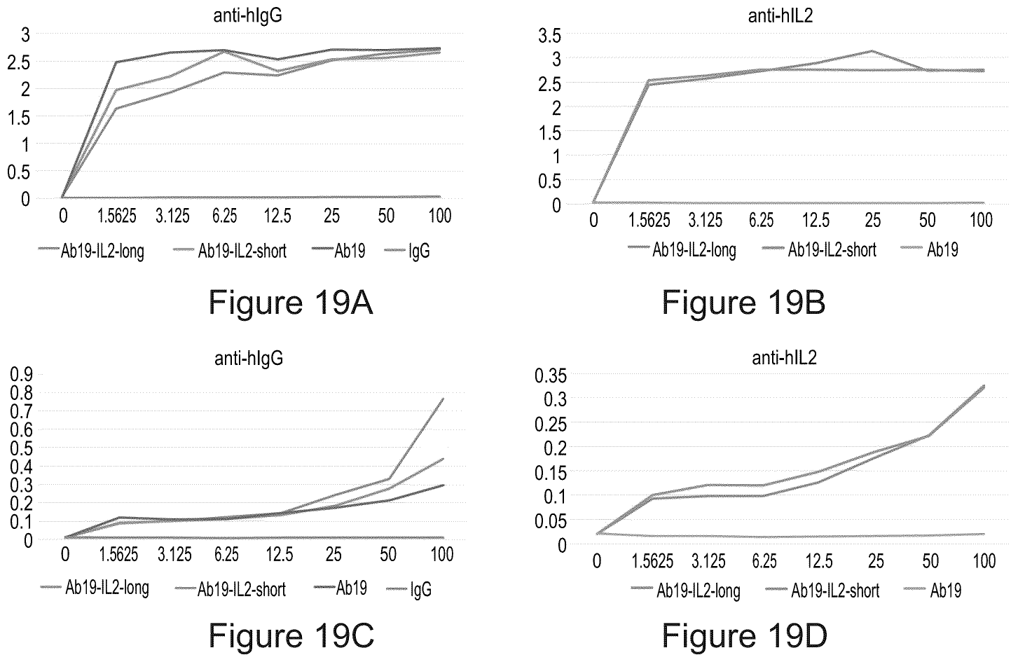

The binding property of the generated antibodies to TREM2 was tested (FIGS. 19A-D).

The conjugated antibodies were shown to bind both recombinant TREM2 (FIG. 19A) and soluble TREM2 (FIG. 19D) similar to the recombinant anti-hTREM2 antibody. In order to confirm the presence of the cytokine on the generated molecules, hIL2 was detected on the hTREM2- bound antibodies (BLG-500302, BioLegend, FIGS. 19B and 19D).

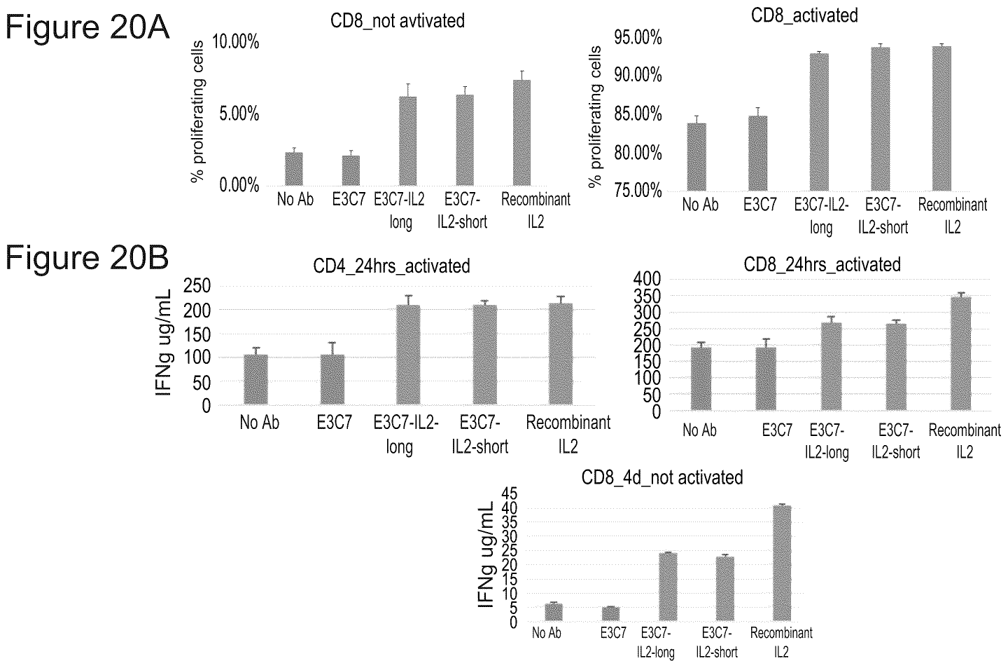

Next, the conjugated antibodies were tested for their ability to activate primary human CD8+ and CD4+ cells (FIGS. 20A-B). The cells were cultured with or without activation in the presence of antibodies (10 μg/ml) or with recombinant proteins (100 ng/ml).

The percentage of proliferating CD8+ cells was found to be elevated by IL2- conjugated antibodies, similar to stimulation with recombinant IL2 (FIG. 20A). This was the case both for activated and not activated CD8+ cells. Stimulation with the recombinant unconjugated anti-hTREM2 did not affect proliferation. IL2- conjugated antibodies stimulated IFN-g secretion by CD8+ cells compared to anti-TREM2 antibody at 24 hrs and 4 days post simulation, similar to stimulation with recombinant IL2 (FIG. 20B). Similarly, IL2- conjugated antibodies stimulated IFN-g secretion by CD4+ cells compared to anti-TREM2 antibody at 24 hrs (FIG. 20B). To validate the ability of the antibodies to deliver the IL2 cytokine to TREM2 expressing macrophages and exert a desired activity (boosting T cell response), an in vitro experiment was performed as follows. Bone marrow derived macrophages (BMDMs) from humanized TREM2 mice were isolated. In two time points these cells were treated with the anti-human TREM2 antibodies (both conjugated and unconjugated to human IL2), and then the ability of the different treated-BMDMs to suppress/activate T cell responses was tested.

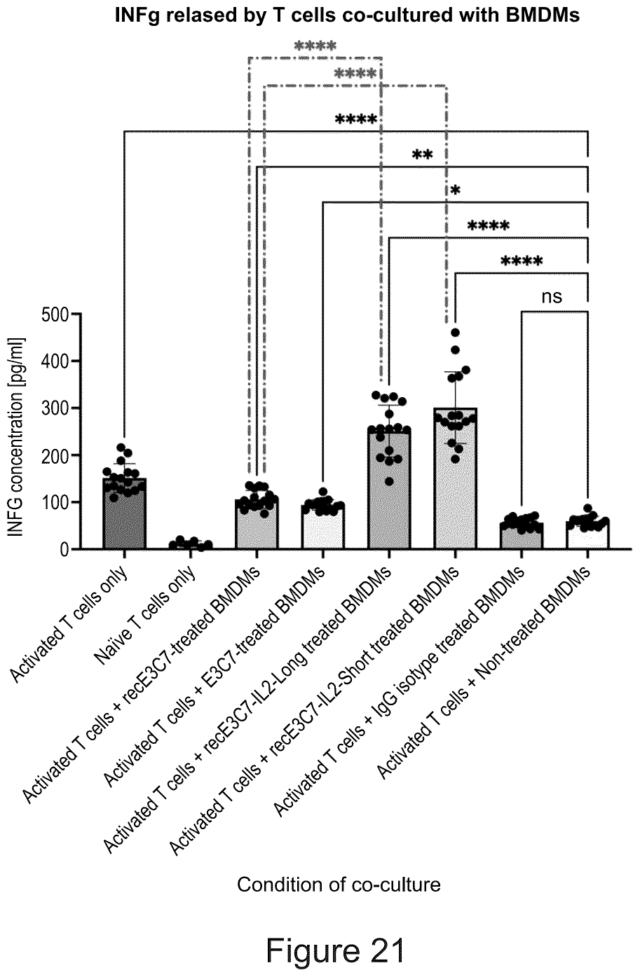

Bone marrow derived macrophages stimulated with IL-4 have immunosuppressive phenotype (M2 statc) and thus inhibit T cell responses. IFNg release by T cells is a direct indicator for their activity and response, the more IFNg they release the more activated they are and vice versa. Thus, the present results show that activated T cells co-cultured with non-treated BMDMs indeed were more inhibited compared to activated T cells only control. Moreover, BMDMs treated with anti-human TREM2 (both recE3C7 and E3C7) inhibited T cells less than the non-treated control and then isotype IgG control and that the IgG isotype control did not have the same effect (FIG. 21). Moreover, the results showed that the conjugated anti-TREM2-IL2 treatment (both short and long form of human IL2) boosted the T cells responses by 4.2-5-fold higher when compared to activated T cells co-cultured with recE3C7 BMDMs (FIG. 21).

### EXAMPLE 6

Targeting Tumor-Associated Macrophages (TAMs) with TREM2 CAR-T Cells

CART cells aimed to recognize and deplete TREM2 expressing myeloid cells were produced. The high-affinity (low nanomolar to picomolar range) recognition domain of the 80E3C7 monoclonal Ab was used in this setting. The results demonstrated that TREM2-CAR T cells are able to initiate an effective cytotoxic response and deplete human TREM2 expressing myeloid cells and immunosuppressive macrophages.

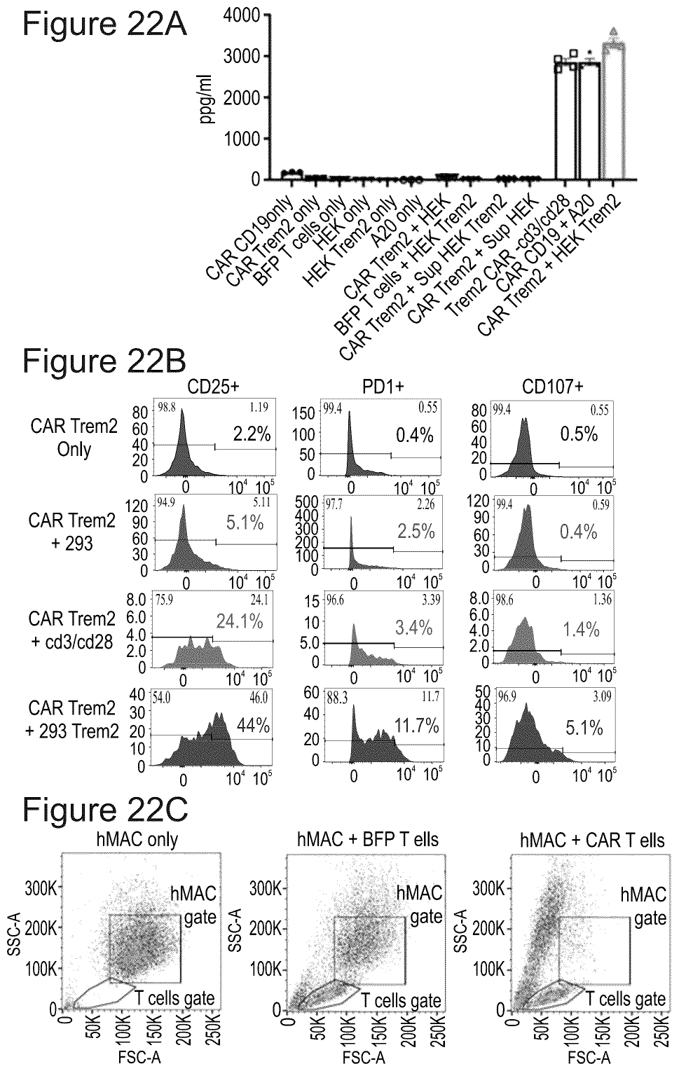

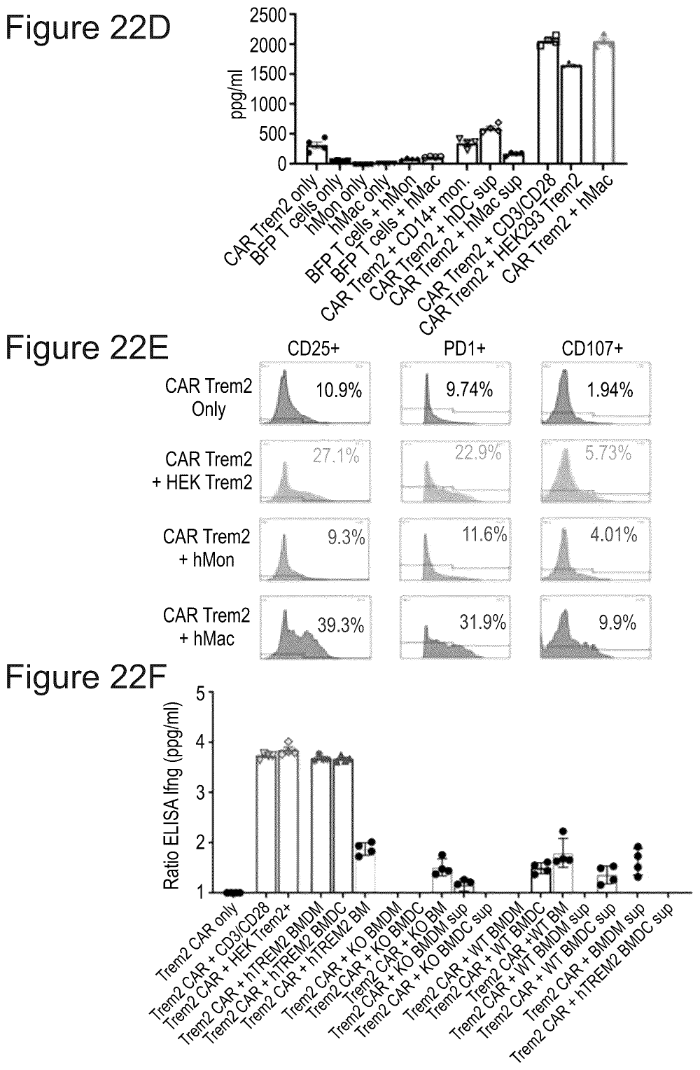

First, to validate TREM2-CAR T cells recognition of TREM2, a TREM2 expressing cell line was used. TREM2-CAR T cells were co-cultured with HEK293 over-expressing TREM2 in a ratio of 1:1 (FIG. 22A-B). As positive controls, activated TREM2-CAR T was used with CD3/CD28 beads and CD19-CAR culture with A20 cell line. Additionally, T cells transduced with a mock CAR (BFP T cells) and HEK293 without TREM2 expression were used. After 24 hours IFN-γ ELISA was performed, which showed a dramatic IFN-γ secretion of the CAR-TREM2 T cells when co-cultured with HEK293 over-expressing TREM2. The results were validated using flow cytometry with markers for activation (CD25 and PD1) and killing (CD107) which show a significant increase matched only to CD19-CAR T cells and bead activation. Non-specific response with HEK293 were not observed. These results showed CAR-TREM2 T cells response to TREM2 is highly specific and potent.

To validate the finding in human settings, CD14+ monocytes were purified from human blood, and the cells were differentiated to TAM-like cells and dendritic cells for one weck. The, TREM2-CAR T cells were cultured with both products and the undifferentiated human CD14+ in a ratio of 1:1 (FIG. 22C-E). As positive controls, activated TREM2-CAR T cells were used with CD3/CD28 beads and cultured with HEK293 TREM2+. As before, T cells transduced with a mock CAR (BFP T cells) were used as a negative control. As shown in FIG. 22C, after 24 hours a complete cradication of all human TAM-like cells. expressing macrophages cultured with TREM2-CAR T cells was observed, while mock T cells did not show any response. IFNg ELISA and flow cytometry analysis were performed again as described above. The present results show also in human setting a high specificity and cytotoxicity ability of the TREM2-CAR T cells in depleting human TAM-like cells.

To further validate specificity and potency of the TREM2-CAR T cells, BMDM and BMDC differentiated from the bone-merrow derived cells of humanized TREM2 mice, TREM2KO mice and wt mice (FIG. 22F) were compared. Similar experimental settings were used as described above. The present results showed a significant potent killing response only when TREM2-CAR T cells were cultured with differentiated cells from humanized TREM2 mice.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An antibody or a fragment thereof comprising an antigen recognition domain capable of binding Triggering Receptor Expressed On Myeloid Cells 2 (TREM2), wherein said antigen recognition domain comprises the complementarity determining regions (CDRs) CDRH1 as set forth by SEQ ID NO: 180, CDRH2 as set forth by SEQ ID NO: 181, CDRH3 as set forth by SEQ ID NO: 182, CDRL1 as set forth by SEQ ID NO: 183, CDRL2 as set forth by the Alanine-Alanine-Serine amino acid sequence and CDRL3 as set forth by SEQ ID NO: 185, or the heavy chain as set forth by SEQ ID NO: 41 and light chain as set forth by SEQ ID NO: 77.

2. A bispecific antibody comprising in at least one arm thereof the antigen recognition domain of claim 1.

3. The antibody of claim 1, having a null or no effector function.

4. The antibody of claim 1 being IgG1.

5. The antibody or fragment thereof of claim 1, being formulated as an antibody drug conjugate (ADC).

6. The antibody or fragment thereof of claim 1, being formulated with a pro-inflammatory cytokine.

7. The antibody or fragment thereof of claim 6, being conjugated to said pro-inflammatory cytokine to form a conjugate.

8. The antibody or fragment thereof claim 7, wherein said conjugate is as set forth in SEQ ID NO: 496 and 498 or 497 and 499.

9. The fragment of the antibody of claim 1 forming a chimeric antigen receptor (CAR).

10. A cell expressing the fragment of the antibody of claim 9.

11. An article of manufacture comprising the antibody or antibody fragment of claim 1.

12. A pharmaceutical composition comprising the antibody or antibody fragment of claim 1 and a pharmaceutically acceptable carrier or diluent.

13. A method of reducing the immune suppressor activity of myeloid cells, the method comprising contacting myeloid cells with an effective amount of the antibody or antibody fragment of claim 1, thereby reducing the immune suppressor activity of myeloid cells.

14. A method of killing myeloid cells expressing TREM2, the method comprising contacting a population of cells comprising contacting TREM2 expressing myeloid cells with an effective amount of cells of claim 10, thereby killing the myeloid cells expressing TREM2.

15. A method of treating cancer in a subject in need thereof, the method comprising administering to the subject an effective amount of the antibody or antibody fragment of claim 1, thereby treating the cancer.

16. A method of treating cancer in a subject in need thereof, the method comprising:
(a) reducing the immune suppressor activity of myeloid cells according to the method of claim 13, wherein said myeloid cells are derived from the subject; and subsequently
(b) transplanting said myeloid cells to the subject, thereby treating the cancer.

17. The method of claim 15, wherein said cancer is a solid cancer.

18. The method of claim 15, further comprising administering to the subject a therapeutically effective amount of a checkpoint inhibitor.

19. The method of claim 15, further comprising administering to the subject a therapeutically effective amount of a Brutons tyrosine kinase (Btk) inhibitor.


SEQUENCE LISTING (510 sequences)

SEQ ID NO: 1 - protein, 230 aa, synthetic construct
  REGION 1..230: note = An exemplary human TREM-2 amino acid sequence
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 470 aa, synthetic construct
  REGION 1..470: note = exemplary GPNMB (Transmembrane glycoprotein NMB ) amino acidsequence
KRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPAS

SEQ ID NO: 3 - DNA, 36 bp, synthetic construct
  misc_feature 1..36: note = T7 RNA polymerase promoter sequence
  source 1..36: mol_type = other DNA
scgattgaggccggtaatacgactcactataggggc

SEQ ID NO: 4 - protein, 180 aa, synthetic construct
  REGION 1..180: note = extracellular domain of human TREM2 fused to the His tag aminoacid sequence
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSHHHHHH

SEQ ID NO: 5 - protein, 504 aa, synthetic construct
  REGION 1..504: note = extracellular domain of human GPNMB fused to the His tag aminoacid sequence
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANHHHHHH

SEQ ID NO: 6 - protein, 357 aa, synthetic construct
  REGION 1..357: note = Antibody heavy Chain nucleic acid sequence
CAGCTGCAGGAGTCTGGACCTGTGCTGGTGAAGCCTGGGGCTTCAGTGAAGATGTCCTGTAAGGCTTCTGGATACACATTCACTGACTACTATATGAACTGGGTGAAGCAGAGCCATGGAAAGAGCCTTGAGTGGATTGGAGTTATTAATCCTTACACCGGTGCTACTAGCTACAACCAGAAGTTCAAGGGCAAGGCCACATTGACTGTTGACAAGTCCTCCAGCACAGCCTACATGGAGCTCAACAGCCTGACATCTGAGGACTCTGCAGTCTATTACTGTGCAAGAGGGACCTACTATAGTAACTACGAGGGGTTTCCTTACTGGGGCCAAGGGACTCTGGTCACTGTCTCTGCA

SEQ ID NO: 7 - protein, 361 aa, synthetic construct
  REGION 1..361: note = Antibody heavy Chain nucleic acid sequence
CAGCTGCAGGAGTCTGGACCTGACCTGGTGAAGCCTGGGGCTTCAGTGAAGATATCCTGTCAGGCTTCTGGCTTCACGTTCACTGACTACTTCGTGAACTGGGTGAGGCAGAGCCATGGAAAGAGCCTTGAGTGGATTGGAGATATTAATCCTAACAATGGTGGTACCACCTACAACCAGAAATTCACGGGCAAGGCCACATTGACTGTCGACAAGTCCTCCAGCACAGCCTACATGGAGCTCCGCAGCCTGACATCTGAGGACTCTGCAGTCTATTACTGTGTAGGAGAGGGGACTTACTACGGTAGAGGCTGGTACTTCGATGTCTGGGGCACAGGGACCACGGTCACCGTCTCCTCAG

SEQ ID NO: 8 - protein, 364 aa, synthetic construct
  REGION 1..364: note = Antibody heavy Chain nucleic acid sequence
CAGCTGCAGGAGTCTGGACCTGAGCTGGTGAAGCCTGGGGCTTCACTGAAGATATCCTGCAAGGCTTCTGGCTACACCTTCACTGACTACTATATAAACTGGGTGAAGCAGAGGCCTGGACAGGGACTTGAGTGGATTGGATGGATTTTTCCTGGAACTGGTAGTACTTCCTACAATGAGCAGTTCAGGGGCAAGGCCACACTTACTATAGACAAATCCTCCAGCACAGCCTACATGTTGCTCAGCAGCCTGACCTCTGAGGACTCTGCGGTCTATTTCTGTGCAAGTCCTATCTACTATGGTAACCACTATGAGGGCTACTTTGACTACTGGGGCCAAGGCACCACTCTCACAGTCTCCTCAG

SEQ ID NO: 9 - protein, 358 aa, synthetic construct
  REGION 1..358: note = Antibody heavy Chain nucleic acid sequence
CAGCTGCAGGAGTCTGGACCTGAGCTGGTGAAGCCTGGGGCTTCAGTGAAGATATCCTGCAAGGCTTCTGGTTACTCATTCACTGGCTACTACATGAACTGGGTGAAGCAAAGTCCTGAAAAGAGCCTTGAGTGGATTGGACAGATTAATCCTAGCACTGGTACTACTACCTACAATCAGAAGTTCAAGGCCAAGGCCACATTGACTGTAGACAAATCCTCCAGCACAGCCTACATGCAGCTCAAGAGCCTGACATCTGAGGACTCTGCAGTCTATTACTGTGCAAGGGGGGGTTATTACTACGGTAGTAGCTACGTAGGGTACTGGGGCCAAGGCACCACTCTCACAGTCTCCTCAG

SEQ ID NO: 10 - protein, 363 aa, synthetic construct
  REGION 1..363: note = Antibody heavy Chain nucleic acid sequence
CAGGTGCAGCTGCAGGAGTCTGGACCTGAGCTGGTGAAGCCTGGGGCCTCAGTGAAGATTTCCTGCAAGGCTTCTGGCTACATATTCAGTTACTCCTGGATGAACTGGGTGAAACAGAGGCCTGGAAAGGGTCTTGAGTGGATTGGACGGATTTATCCTGGAGATGGAGATGATGACTACAATGGGAAGTTCAAGGGCAAGGCCACACTGACTGCAGACAAATCCTCCAACACAGCCTACATGCAACTCACCAGCCTGACATCTGAGGACTCTGCGGTCTACTTCTGTGCAAGACTTCACATCTATGATGGTTATTACGTCTCTGTTTATTGGGGCCAAGGGACTCTGGTCACTGTCTCGGCA

SEQ ID NO: 11 - protein, 360 aa, synthetic construct
  REGION 1..360: note = Antibody heavy Chain nucleic acid sequence
CAGGTCCAACTACAGCAGCCTGGGGCTGAGCCACTGAAGCCTGGGGCTTCAGTGAAGATGTCCTGCGAGGCTTCTGGCTACACCTTCACCAACTTCTGGATAACCTGGATGAAGCTGAGGCCTGGACAAGGCCTTGAGTGGATTGGAGATATTTATCCTGGTACTGGTAATACCAACTATAATGAGAAGTTCAAGAGCAGGGCCACACTGACTGTGGACACATCCTCCACCACAGCCTACATGCAGCTCAGCAGCCTGACATCTGAGGACTCAGCGGTCTATTACTGTGCAAGAGAGGCCTACTATACTAACCCCGGGTTTGCTTACTGGGGCCAAGGGACTCTGGTCACTGTCTCGGCA

SEQ ID NO: 12 - protein, 348 aa, synthetic construct
  REGION 1..348: note = Antibody heavy Chain nucleic acid sequence
GAGGTGCAGCTGCAGGAGTCTGGACCTGAGCTGGTGAAGCCTGGGGCTTCAGTGAAGATGTCCTGCAAGGCTTCTGGATACACATTCACTGACTACAACATGCACTGGGTGAAGCAGAGCCATGGAAAGAGCCTTGAGTGGATTGGATATATTTACCCTAACAATGGTGGTACTGGTTACAACCAGAAGTTCAAGGGCAAGGCCACATTGACTGTAAACAAGTCCTCCAGCACAGCCTACATGGAGCTCCGCAGCCTGACATCGGAGGATTCTGCAGTCTATTACTGTGCAAGATCTGGGCGGGGGTTTGCTTACTGGGGCCAAGGGACTCTGGTCACTGTCTCTGCA

SEQ ID NO: 13 - protein, 348 aa, synthetic construct
  REGION 1..348: note = Antibody heavy Chain nucleic acid sequence
CAGCTGCAGGAGTCTGGACCTGAGCAGGTGAAGCCTGGGGCTTCAGTGAAGATGTCCTGCAGGACTTCTGGATACACATTCACTGCCTACAACATGCACTGGGTGAAACAGAGACTTGGAAAGAGCCTTGAGTGGATTGGATATATTAGGCCTACCAATGGAGATACTCGCTACAATCGGAGGTTCAGGGACAAGGCCACATTGACTGTTGACAAGTCCTCCAGCACAACCTACATGGAGCTCCGCAGCCTGACATCGGAGGATTCTGCAGTCTATTACTGTACAAGATCAACTACGGTAGCCTCTTTTGACTACTGGGGCCAAGGCACCACTCTCACAGTCTCCTCA

SEQ ID NO: 14 - protein, 360 aa, synthetic construct
  REGION 1..360: note = Antibody heavy Chain nucleic acid sequence
CAGGTGCAGCTGCAGGAGTCTGGGGCTGAGCCACTGAAGCCTGGGGCTTCAGTGAAGATGTCCTGCGAGGCTTCTGGCTACACCTTCACCAACTTCTGGATAACCTGGATGAAGCTGAGGCCTGGACAAGGCCTTGAGTGGATTGGAGATATTTATCCTGGTACTGGTAATACCAACTATAATGAGAAGTTCAAGAGCAGGGCCACACTGACTGTGGACACATCCTCCACCACAGCCTACATGCAGCTCAGCAGCCTGACATCTGAGGACTCAGCGGTCTATTACTGNGCAAGAGAGGCCTACTATACTAACCCCGGGTTTGCTTACTGGGGCCACGGGACTCTGGTCACTGTCTCTGCA

SEQ ID NO: 15 - protein, 363 aa, synthetic construct
  REGION 1..363: note = Antibody heavy Chain nucleic acid sequence
CAGGTGCAGCTGCAGGAGTCTGGACCTGAGCTGGTGAAGCCTGGGGCCTCAGTGAAGATTTCCTGCAAGGCTTCTGGCTACATATTCAGTTACTCCTGGATGAACTGGGTGAAACAGAGGCCTGGAAAGGGTCTTGAGTGGATTGGACGGATTTATCCTGGAGATGGAGATGCTGACTACAATGGGAAGTTCAAGGGCAAGGCCACACTGACTGCAGACAAATCCTCCAACACAGCCTACATGCAACTCACCAGCCTGACATCTGAGGACTCTGCGGTCTACTTCTGTGCAAGACTTCACATCTATGATGGTTATTACGTCTCTGTTTATTGGGGCCAAGGGACTCTGGTCACTGTCTCTGCA

SEQ ID NO: 16 - protein, 360 aa, synthetic construct
  REGION 1..360: note = Antibody heavy Chain nucleic acid sequence
CAGGTCCAACTGCAGCAGCCTGGGGCTGAGCCACTGACGCCTGGGGCTTCAGTGAAGATGTCCTGCGAGGCTTCTGGCTACACCTTCACAAATTTCTGGATAACCTGGGTGAAACTGAGGCCTGGACAAGGCCTTGAGTGGATTGGAGATATTTATCCTGGTAGTGGTAATACCAACTACAATGAGAAGTTCAAGAGCAGGGCCACACTGACTGTGGACACTTCCTCCACCACAGCCTACATGCAGCTCAGCAGCCTGACATCTGAGGACTCAGCGGTCTACTACTGTGCAAGAGAGGCCTACTATACTAACCCCGGGTTTGCTTACTGGGGCCACGGGACTCTGGTCACTGTCTCTGCA

SEQ ID NO: 17 - protein, 360 aa, synthetic construct; identical to SEQ ID NO: 16
  REGION 1..360: note = Antibody heavy Chain nucleic acid sequence

SEQ ID NO: 18 - protein, 363 aa, synthetic construct
  REGION 1..363: note = Antibody heavy Chain nucleic acid sequence
GAGCTGCAGCTGCAGGAGTCTGGACCTGTGCTGGTGAAGCCTGGGGCTTCAGTGAAGATGTCCTGTAAGGCTTCTGGATACACATTCACTGACTACTATATGAACTGGGTGAAGCAGAGCCATGGAAAGAGCCTTGAGTGGATTGGAGTTATTAATCCTTACACCGGTGCTACTAGCTACAACCAGAAGTTCAAGGGCAAGGCCACATTGACTGTTGACAAGTCCTCCAGCACAGCCTACATGGAGCTCAACAGCCTGACATCTGAGGACTCTGCAGTCTATTACTGTGCAAGAGGGACCTACTATAGTAACTACGAGGGGTTTCCTTACTGGGGCCAAGGGACTCTGGTCACTGTCTCTGCA

SEQ ID NO: 19 - protein, 363 aa, synthetic construct
  REGION 1..363: note = Antibody heavy Chain nucleic acid sequence
GAGGTGCAGCTGCAGGAGTCTGGACCTGTGCTGGTGAAGCCTGGGGCTTCAGTGAAGATGTCCTGTAAGGCTTCTGGATACACATTCACTGACTACTATATGAACTGGGTGAAGCAGAGCCATGGAAAGAGCCTTGAGTGGATTGGAGTTATTAATCCTTACACCGGTGCTACTAGCTACAACCAGAAGTTCAAGGGCAAGGCCACATTGACTGTTGACAAGTCCTCCAGCACAGCCTACATGGAGCTCAACAGCCTGACATCTGAGGACTCTGCAGTCTATTACTGTGCAAGAGGGACCTACTATAGTAACTACGAGGGGTTTCCTTACTGGGGCCAAGGGACTCTGGTCACTGTCTCTGCA

SEQ ID NO: 20 - protein, 369 aa, synthetic construct
  REGION 1..369: note = Antibody heavy Chain nucleic acid sequence
CAGGTGCAGCTGGAGCAGTCAGGACCTGAGCTGGTGAAGCCTGGGGCTTCACTGAAGATATCCTGCAAGGCTTCTGGCTACACCTTCACTGACTACTATATAAACTGGGTGAAGCAGAGGCCTGGACAGGGACTTGAGTGGATTGGATGGATTTTTCCTGGAACTGGTAGTACTTCCTACAATGAGCAGTTCAGGGGCAAGGCCACACTTACTATAGACAAATCCTCCAGCACAGCCTACATGTTGCTCAGCAGCCTGACCTCTGAGGACTCTGCGGTCTATTTCTGTGCAAGTCCTATCTACTATGGTAACCACTATGAGGGCTACTTTGACTACTGGGGCCAAGGCACCACTCTCACAGTCTCCTCA

SEQ ID NO: 21 - protein, 357 aa, synthetic construct
  REGION 1..357: note = Antibody heavy Chain nucleic acid sequence
CAGGTGCAGCTGCAGGAGTCTGGACCTGAACTGGTGAAGCCTGGGGCTTCAGTGAAGATATCCTGCAAGGCTTCTGGCTACAGCTTCACAAGCTTCTTTTTACATTGGGTGAGTCAGAGGCCTGGACAGGGACTTGCGTGGATTGGAAGGATTCATCCTGGAAGTGGTAACACTAAGTACAATGAGAAGTTCAAGGGCAAGGCCTCACTGACGGCAGACACATCCTCCAGCACTGCCTACATACAACTCAGCAGCCTTCCATCTGATGACTCTGCGGTCTATTACTGTGCAAGAGAGGGATTTCAATACGACTACTTTCCTTATTGGGGCCAAGGGACTCTGGTCACTGTCTCTGCA

SEQ ID NO: 22 - protein, 348 aa, synthetic construct
  REGION 1..348: note = Antibody heavy Chain nucleic acid sequence
GAGGTGCAGCTGCAAGAGTCTGGACCTGAGCTGGTGAAGCCTGGGGCTTCAGTGAAGATGTCCTGCAAGGCTTCTGGATACACATTCACTGACTACAACATGCACTGGGTGAAGCAGAGCCATGGAAAGAGCCTTGAGTGGATTGGATATATTTACCCTAACAATGGTGGTACTGGTTACAACCAGAAGTTCAAGGGCAAGGCCACATTGACTGTAAACAAGTCCTCCAGCACAGCCTACATGGAGCTCCGCAGCCTGACATCGGAGGATTCTGCAGTCTATTACTGTGCAAGATCTGGGCGGGGGTTTGCTTACTGGGGCCAAGGGACTCTGGTCACTGTCTCTGCA

SEQ ID NO: 23 - protein, 360 aa, synthetic construct
  REGION 1..360: note = Antibody heavy Chain nucleic acid sequence
CAGGTCCAACTGCAGCAGCCTGGGGCTGAGCCACTGAAGCCTGGGGCTTCAGTGAAGATGTCCTGCGAGGCTTCTGGCTACACCTTCACCAACTTCTGGATAACCTGGATGAAGCTGAGGCCTGGACAAGGCCTTGAGTGGATTGGAGATATTTATCCTGGTACTGGTAATACCAACTATAATGAGAAGTTCAAGAGCAGGGCCACACTGACTGTGGACACATCCTCCACCACAGCCTACATGCAGCTCAGCAGCCTGACATCTGAGGACTCAGCGGTCTATTACTGTGCAAGAGAGGCCTACTATACTAACCCCGGGTTTGCTTACTGGGGCCACGGGACTCTGGTCACTGTCTCTGCA

SEQ ID NO: 24 - protein, 119 aa, synthetic construct
  REGION 1..119: note = Antibody heavy Chain amino acid sequence
QLQESGPVLVKPGASVKMSCKASGYTFTDYYMNWVKQSHGKSLEWIGVINPYTGATSYNQKFKGKATLTVDKSSSTAYMELNSLTSEDSAVYYCARGTYYSNYEGFPYWGQGTLVTVSA

SEQ ID NO: 25 - protein, 120 aa, synthetic construct
  REGION 1..120: note = Antibody heavy Chain amino acid sequence
QLQESGPDLVKPGASVKISCQASGFTFTDYFVNWVRQSHGKSLEWIGDINPNNGGTTYNQKFTGKATLTVDKSSSTAYMELRSLTSEDSAVYYCVGEGTYYGRGWYFDVWGTGTTVTVSS

SEQ ID NO: 26 - protein, 121 aa, synthetic construct
  REGION 1..121: note = Antibody heavy Chain amino acid sequence
QLQESGPELVKPGASLKISCKASGYTFTDYYINWVKQRPGQGLEWIGWIFPGTGSTSYNEQFRGKATLTIDKSSSTAYMLLSSLTSEDSAVYFCASPIYYGNHYEGYFDYWGQGTTLTVSS

SEQ ID NO: 27 - protein, 119 aa, synthetic construct
  REGION 1..119: note = Antibody heavy Chain amino acid sequence
QLQESGPELVKPGASVKISCKASGYSFTGYYMNWVKQSPEKSLEWIGQINPSTGTTTYNQKFKAKATLTVDKSSSTAYMQLKSLTSEDSAVYYCARGGYYYGSSYVGYWGQGTTLTVSS

SEQ ID NO: 28 - protein, 121 aa, synthetic construct
  REGION 1..121: note = Antibody heavy Chain amino acid sequence
QVQLQESGPELVKPGASVKISCKASGYIFSYSWMNWVKQRPGKGLEWIGRIYPGDGDDDYNGKFKGKATLTADKSSNTAYMQLTSLTSEDSAVYFCARLHIYDGYYVSVYWGQGTLVTVSA

SEQ ID NO: 29 - protein, 120 aa, synthetic construct
  REGION 1..120: note = Antibody heavy Chain amino acid sequence
QVQLQQPGAEPLKPGASVKMSCEASGYTFTNFWITWMKLRPGQGLEWIGDIYPGTGNTNYNEKFKSRATLTVDTSSTTAYMQLSSLTSEDSAVYYCAREAYYTNPGFAYWGQGTLVTVSA

SEQ ID NO: 30 - protein, 116 aa, synthetic construct
  REGION 1..116: note = Antibody heavy Chain amino acid sequence
EVQLQESGPELVKPGASVKMSCKASGYTFTDYNMHWVKQSHGKSLEWIGYIYPNNGGTGYNQKFKGKATLTVNKSSSTAYMELRSLTSEDSAVYYCARSGRGFAYWGQGTLVTVSA

SEQ ID NO: 31 - protein, 116 aa, synthetic construct
  REGION 1..116: note = Antibody heavy Chain amino acid sequence
QLQESGPEQVKPGASVKMSCRTSGYTFTAYNMHWVKQRLGKSLEWIGYIRPTNGDTRYNRRFRDKATLTVDKSSSTTYMELRSLTSEDSAVYYCTRSTTVASFDYWGQGTTLTVSS

SEQ ID NO: 32 - protein, 120 aa, synthetic construct
  REGION 1..120: note = Antibody heavy Chain amino acid sequence
  SITE 96: note = misc_feature - Xaa can be any naturally occurring amino acid
QVQLQESGAEPLKPGASVKMSCEASGYTFTNFWITWMKLRPGQGLEWIGDIYPGTGNTNYNEKFKSRATLTVDTSSTTAYMQLSSLTSEDSAVYYXAREAYYTNPGFAYWGHGTLVTVSA

SEQ ID NO: 33 - protein, 121 aa, synthetic construct
  REGION 1..121: note = Antibody heavy Chain amino acid sequence
QVQLQESGPELVKPGASVKISCKASGYIFSYSWMNWVKQRPGKGLEWIGRIYPGDGDADYNGKFKGKATLTADKSSNTAYMQLTSLTSEDSAVYFCARLHIYDGYYVSVYWGQGTLVTVSA

SEQ ID NO: 34 - protein, 120 aa, synthetic construct
  REGION 1..120: note = Antibody heavy Chain amino acid sequence
QVQLQQPGAEPLTPGASVKMSCEASGYTFTNFWITWVKLRPGQGLEWIGDIYPGSGNTNYNEKFKSRATLTVDTSSTTAYMQLSSLTSEDSAVYYCAREAYYTNPGFAYWGHGTLVTVSA

SEQ ID NO: 35 - protein, 120 aa, synthetic construct; identical to SEQ ID NO: 34
  REGION 1..120: note = Antibody heavy Chain amino acid sequence

SEQ ID NO: 36 - protein, 121 aa, synthetic construct
  REGION 1..121: note = Antibody heavy Chain amino acid sequence
ELQLQESGPVLVKPGASVKMSCKASGYTFTDYYMNWVKQSHGKSLEWIGVINPYTGATSYNQKFKGKATLTVDKSSSTAYMELNSLTSEDSAVYYCARGTYYSNYEGFPYWGQGTLVTVSA

SEQ ID NO: 37 - protein, 121 aa, synthetic construct
  REGION 1..121: note = Antibody heavy Chain amino acid sequence
EVQLQESGPVLVKPGASVKMSCKASGYTFTDYYMNWVKQSHGKSLEWIGVINPYTGATSYNQKFKGKATLTVDKSSSTAYMELNSLTSEDSAVYYCARGTYYSNYEGFPYWGQGTLVTVSA

SEQ ID NO: 38 - protein, 123 aa, synthetic construct
  REGION 1..123: note = Antibody heavy Chain amino acid sequence
QVQLEQSGPELVKPGASLKISCKASGYTFTDYYINWVKQRPGQGLEWIGWIFPGTGSTSYNEQFRGKATLTIDKSSSTAYMLLSSLTSEDSAVYFCASPIYYGNHYEGYFDYWGQGTTLTVSS

SEQ ID NO: 39 - protein, 119 aa, synthetic construct
  REGION 1..119: note = Antibody heavy Chain amino acid sequence
QVQLQESGPELVKPGASVKISCKASGYSFTSFFLHWVSQRPGQGLAWIGRIHPGSGNTKYNEKFKGKASLTADTSSSTAYIQLSSLPSDDSAVYYCAREGFQYDYFPYWGQGTLVTVSA

SEQ ID NO: 40 - protein, 116 aa, synthetic construct; identical to SEQ ID NO: 30
  REGION 1..116: note = Antibody heavy Chain amino acid sequence

SEQ ID NO: 41 - protein, 120 aa, synthetic construct
  REGION 1..120: note = Antibody heavy Chain amino acid sequence
QVQLQQPGAEPLKPGASVKMSCEASGYTFTNFWITWMKLRPGQGLEWIGDIYPGTGNTNYNEKFKSRATLTVDTSSTTAYMQLSSLTSEDSAVYYCAREAYYTNPGFAYWGHGTLVTVSA

SEQ ID NO: 42 - protein, 321 aa, synthetic construct
  REGION 1..321: note = Antibody light Chain nucleic acid sequence
GACATCCAGATGACCCAGTCTCCATCCTCCTTATCTGCCTCTCTGGGAGAAAGAGTCAGTCTCACTTGTCGGGCAAGTCAGGAAATTAGTGGTTACTTAAGCTGGCTTCAGCAGAAACCAGATGGAACTATTAAACGCCTGATCTACGCCGCATCCACTTTAGATTCTGGTGTCCCAAAAAGGTTCAGTGGCAGTAGGTCTGGGTCAGATTATTCTCTCACCATCAGCAGCCTTGAGTCTGAAGATTTTGCAGACTATTACTGTCTACAATATGCTAGTTATCCGTACACGTTCGGAGGGGGGACCAAGCTGGAAATAAAA

SEQ ID NO: 43 - protein, 331 aa, synthetic construct
  REGION 1..331: note = Antibody light Chain nucleic acid sequence
GACATTGTGCTGACACAGTCTCCTGCTTCCTTAGCTGTATCTCTGGGGCAGAGGGCCACCATCTCATACAGGGCCAGCAAAAGTGTCAGTACATCTGGCTATAGTTATATGCACTGGAACCAACAGAAACCAGGACAGCCACCCAGACTCCTCATCTATCTTGTATCCAACCTAGAATCTGGGGTCCCTGCCAGGTTCAGTGGCAGTGGGTCTGGGACAGACTTCACCCTCAACATCCATCCTGTGGAGGAGGAGGATGCTGCAACCTATTACTGTCAGCACATTAGGGAGCTTTACACGTTCGGAGGGGGGACCAAGCTGGAAATAAAAC

SEQ ID NO: 44 - protein, 330 aa, synthetic construct
  REGION 1..330: note = Antibody light Chain nucleic acid sequence
GACATTGTGCTGACACAGTCTCCTGCTTCCTTAGCTGTATCTCTGGGGCAGAGGGCCACCATCTCATACAGGGCCAGCAAAAGTGTCAGTACATCTGGCTATAGTTATATGCACTGGAACCAACAGAAACCAGGACAGCCACCCAGACTCCTCATCTATCTTGTATCCAACCTAGAATCTGGGGTCCCTGCCAGGTTCAGTGGCAGTGGGTCTGGGACAGACTTCACCCTCAACATCCATCCTGTGGAGGAGGAGGATGCTGCAACCTATTACTGTCAGCACATTAGGGAGCTTTACACGTTCGGAGGGGGGACCAAGCTGGAAATAAAA

SEQ ID NO: 45 - protein, 330 aa, synthetic construct; identical to SEQ ID NO: 44
  REGION 1..330: note = Antibody light Chain nucleic acid sequence

SEQ ID NO: 46 - protein, 330 aa, synthetic construct; identical to SEQ ID NO: 44
  REGION 1..330: note = Antibody light Chain nucleic acid sequence

SEQ ID NO: 47 - protein, 336 aa, synthetic construct
  REGION 1..336: note = Antibody light Chain nucleic acid sequence
GATGTTGTGATGACACAAACTACACTCTCCCTGCCTGTCAGTCTTGGAGATCAAGCCTCCATCTCTTGCAGATCTAGTCAGACCATTGTGCATAGTAATGGAAACACCTATTTAGAATGGTACCTGCAGAAACCAGGCCAGTCTCCAAAGCTCCTGATCTTCAAAGTTTCCAACCGATTTTCTGGGGTCCCAGACAGGTTCAGTGGCAGTGGATCAGGGACAGATTTCACACTCAAGATCAGCAGAGTGGGGGCTGAGGATCTGGGAATTTATTACTGCTTTCAAGGTTCACATGTTCCGTACACGTTCGGAGGGGGGACCAAGCTGGAAATAAAA

SEQ ID NO: 48 - protein, 321 aa, synthetic construct
  REGION 1..321: note = Antibody light Chain nucleic acid sequence
GACATTGTGATGACTCAGTCTCCAGCCAGCCTGTCTGTGACTCCAGGAGATAGAGTCTCTCTTTCCTGCAGGGCCAGCCAGAGTATTAGCGACTACTTACACTGGTATCAACAAAAATCACATGAGTCTCCAAGGCTGCTCATCAAATATGCTTCCCAATCCATCTCTGGGATCCCCTCCAGGTTCAGTGGCAGTGGATCAGGGTCAGATTTCACTCTCAGTATCAACAGTGTGGAACCTGAAGATGTTGGAGTGTATTACTGTCAAAATGGTCACAGCTTTCCGTACACGTTCGGAGGGGGGACCAAGCTGGAAATAAAA

SEQ ID NO: 49 - protein, 339 aa, synthetic construct
  REGION 1..339: note = Antibody light Chain nucleic acid sequence
GACATTGTGATGACACAAACTACATCCTCCCTAACTGTGTCAGTTGGACAGAAGATTACTCTGAACTGCAAGTCCAGTCAGAGCCTTTTATATACTAACAATCAAAAGAACTTCTTGGCCTGGTACCAGCAGAAACCAGGGCAGTCTCCTAAACTGCTGATTTACTGGGCATCCACTAGGGATTCTGGGGTCCCTGATCGCTTCACAGGCAGTGGATCTGGGACAGATTTCACTCTCACCATCACCAGTGTGATGGCTGAAGACCTGGCAGTTTATTTCTGTCAGCAATATTATGACTTTCCTCCGACGTTCGGTGGAGGCACCAAGCTGGAAATAAAA

SEQ ID NO: 50 - protein, 330 aa, synthetic construct; identical to SEQ ID NO: 44
  REGION 1..330: note = Antibody light Chain nucleic acid sequence

SEQ ID NO: 51 - protein, 321 aa, synthetic construct
  REGION 1..321: note = Antibody light Chain nucleic acid sequence
GATATCCAGATGACACAAACTACATCCTGCCTGTCTGCCTCTCTGGGAGACAGAGTCACCATCAGTTGCAGGGCAAGTCAGGACATTAACAATTATTTAAGCTGGTATCAGCAGAAACCAGATGGAACTGTTAAACTCCTGATCTACTACACATCAAAATTACACTCAGGAGTCCCATCAAGGTTCAGTGGCAGTGGGTCTGGAACAGATTATTCTCTCACCATTGGCAACCTGGAGCAAGAAGATGTTGCCACTTACTTTTGCCAACAGGGTGATACGCTTCCGTGGACGTTCGGTGGAGGCACCAGGCTGGAAATCAAA

SEQ ID NO: 52 - protein, 336 aa, synthetic construct
  REGION 1..336: note = Antibody light Chain nucleic acid sequence
GATGTTTTGATGACCCAAAGTCCACTCTCCCTGCCTGTCAATCTTGGAGATCAAGCCTCCATCTCTTGCAAATCTAGTCAGAACATTGTACATAGTAATGGAAACACCTATTTAGAATGGTACTTGCAGAGACCAGGCCAGTCTCCAAAGCTCCTTATCTACAAAGTTTCCAACCGATTTTCTGGGGTCCCAGACAGGTTCAGTGGCAGTGGATCAGGGACAGATTTCACACTCAAGATCAGCAGAGTGGAGGCTGAGGATCTGGGACTTTATTACTGCTTTCAAGGTTCACATGTTCCGTACACGTTCGGAGGGGGGACCAAGCTGGAGATAAAA

SEQ ID NO: 53 - protein, 330 aa, synthetic construct
  REGION 1..330: note = Antibody light Chain nucleic acid sequence
GACATTGTGCTGACACAGTCTCCTGCTTCCTTAGCTGTATCTCTGGGGCAGAGGGCCACCATCTCATACAGGGCCAGCAAAAGTGTCAGTACATCTGGCTATAGTTATATGCACTGGAACCAACAGAAACCAGGACAGCCACCCAGACTCCTCATCTATCTTGTATCCAACCTAGAATCTGGGGTCCCTGCCAGGTTCAGTGGCAGTGGGTCTGGGACAGACTTCACCCTCAACATCCATCCTGTGGAGGAGGAGGATGCTGCAACCTATTACTGTCAGCACATTAGGGAGCTGTACACGTTCGGAGGGGGGACCAAGCTGGAAATAAAA

SEQ ID NO: 54 - protein, 321 aa, synthetic construct; identical to SEQ ID NO: 42
  REGION 1..321: note = Antibody light Chain nucleic acid sequence

SEQ ID NO: 55 - protein, 321 aa, synthetic construct; identical to SEQ ID NO: 42
  REGION 1..321: note = Antibody light Chain nucleic acid sequence

SEQ ID NO: 56 - protein, 331 aa, synthetic construct; identical to SEQ ID NO: 43
  REGION 1..331: note = Antibody light Chain nucleic acid sequence

SEQ ID NO: 57 - protein, 336 aa, synthetic construct
  REGION 1..336: note = Antibody light Chain nucleic acid sequence
GATGTTTTGATGAGCCAAACTCCACTCTCCCTGCCTGTCAGTCGTGGAGATCAAGCCTACATCTCTCGCAGATCTAGTCAGCCCATTGTGCATAGTAATGGAAACACCTATTTAGAATGGTACCTGCAGAAAGCAGGCCAGTCTCCAAAGCTCCTGATCTTCAAAGTTTGCAACCGATTTTCTGGGGTCCCAGACAGGTTCAGTGGCAGTGGATCAGGGACAGATTTCACACTCAAGATCAGCAGAGTGGGGGCTGAGGATCTGGGAATTTATTACTGCTTTCAAGGTATGCAGGTAGCGTACACGTTCGGAGGGGGGACCAAGCTGGAAATAAAA

SEQ ID NO: 58 - protein, 339 aa, synthetic construct
  REGION 1..339: note = Antibody light Chain nucleic acid sequence
GATATTGTGATGACACAATCTACATCCTCCCTGACTGTGACAGCAGGAGAGAAGGTCACTATGAGCTGCAAGTCCAGTCAGAGTCTGTTAAACAGTGGAAATCAAAAGAACTATTTGACCTGGTACCAGCAGAAACCAGGGCAGCCTCCTAAACTGTTGATCTACTGGGCATCCAGTAGGGAATCTGGGGTCCCTGATCGCTTCACAGGCAGAGGATCTGGAACAGATTTTACTCTCACCATCAGCAGTGTGCAGGCTGAAGACCTGGCAGTTTATTACTGTCAGAATGATTATAGTTATCCATTCACGTTCGGCTCGGGGACAAAGCTGGAAATAAAA

SEQ ID NO: 59 - protein, 336 aa, synthetic construct
  REGION 1..336: note = Antibody light Chain nucleic acid sequence
GATGTTTTGATGACCCAAAGTCCGCTCTCCCTGCCTGTCAATCTTGGAGATCAAGCCTCCATCTCTTGCAAATCTAGCCAGAACATTGTACATAGTAATGGAAACACCTATTTAGAATGGTACCTGCAGAGACCAGGCCAGTCTCCAAAGCTCCTTATCTACAAAGTTTCCAACCGATTTTCTGGGGTCCCAGACAGGTTCAGTGGCAGTGGATCAGGGACAGATTTCACACTCAAGATCAGCAGAGTGGAGGCTGAGGATCTGGGAGTTTATTACTGCTTTCAAGGTTCACATGTTCCGTACACGTTCGGAGGGGGGACCAAGCTGGAAATAAAA

SEQ ID NO: 60 - protein, 107 aa, synthetic construct
  REGION 1..107: note = Antibody light Chain amino acid sequence
DIQMTQSPSSLSASLGERVSLTCRASQEISGYLSWLQQKPDGTIKRLIYAASTLDSGVPKRFSGSRSGSDYSLTISSLESEDFADYYCLQYASYPYTFGGGTKLEIK

SEQ ID NO: 61 - protein, 110 aa, synthetic construct
  REGION 1..110: note = Antibody light Chain amino acid sequence
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELYTFGGGTKLEIK

SEQ ID NO: 62 - protein, 110 aa, synthetic construct; identical to SEQ ID NO: 61
  REGION 1..110: note = Antibody light Chain amino acid sequence

SEQ ID NO: 63 - protein, 110 aa, synthetic construct; identical to SEQ ID NO: 61
  REGION 1..110: note = Antibody light Chain amino acid sequence

SEQ ID NO: 64 - protein, 110 aa, synthetic construct; identical to SEQ ID NO: 61
  REGION 1..110: note = Antibody light Chain amino acid sequence

SEQ ID NO: 65 - protein, 112 aa, synthetic construct
  REGION 1..112: note = Antibody light Chain amino acid sequence
DVVMTQTTLSLPVSLGDQASISCRSSQTIVHSNGNTYLEWYLQKPGQSPKLLIFKVSNRFSGVPDRFSGSGSGTDFTLKISRVGAEDLGIYYCFQGSHVPYTFGGGTKLEIK

SEQ ID NO: 66 - protein, 107 aa, synthetic construct
  REGION 1..107: note = Antibody light Chain amino acid sequence
DIVMTQSPASLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIK

SEQ ID NO: 67 - protein, 113 aa, synthetic construct
  REGION 1..113: note = Antibody light Chain amino acid sequence
DIVMTQTTSSLTVSVGQKITLNCKSSQSLLYTNNQKNFLAWYQQKPGQSPKLLIYWASTRDSGVPDRFTGSGSGTDFTLTITSVMAEDLAVYFCQQYYDFPPTFGGGTKLEIK

SEQ ID NO: 68 - protein, 110 aa, synthetic construct; identical to SEQ ID NO: 61
  REGION 1..110: note = Antibody light Chain amino acid sequence

SEQ ID NO: 69 - protein, 107 aa, synthetic construct
  REGION 1..107: note = Antibody light Chain amino acid sequence
DIQMTQTTSCLSASLGDRVTISCRASQDINNYLSWYQQKPDGTVKLLIYYTSKLHSGVPSRFSGSGSGTDYSLTIGNLEQEDVATYFCQQGDTLPWTFGGGTRLEIK

SEQ ID NO: 70 - protein, 112 aa, synthetic construct
  REGION 1..112: note = Antibody light Chain amino acid sequence
DVLMTQSPLSLPVNLGDQASISCKSSQNIVHSNGNTYLEWYLQRPGQSPKLLIYKVSNRFSGVPDRFSGSGSGTDFTLKISRVEAEDLGLYYCFQGSHVPYTFGGGTKLEIK

SEQ ID NO: 71 - protein, 110 aa, synthetic construct; identical to SEQ ID NO: 61
  REGION 1..110: note = Antibody light Chain amino acid sequence

SEQ ID NO: 72 - protein, 107 aa, synthetic construct; identical to SEQ ID NO: 60
  REGION 1..107: note = Antibody light Chain amino acid sequence

SEQ ID NO: 73 - protein, 107 aa, synthetic construct; identical to SEQ ID NO: 60
  REGION 1..107: note = Antibody light Chain amino acid sequence

SEQ ID NO: 74 - protein, 110 aa, synthetic construct; identical to SEQ ID NO: 61
  REGION 1..110: note = Antibody light Chain amino acid sequence

SEQ ID NO: 75 - protein, 112 aa, synthetic construct
  REGION 1..112: note = Antibody light Chain amino acid sequence
DVLMSQTPLSLPVSRGDQAYISRRSSQPIVHSNGNTYLEWYLQKAGQSPKLLIFKVCNRFSGVPDRFSGSGSGTDFTLKISRVGAEDLGIYYCFQGMQVAYTFGGGTKLEIK

SEQ ID NO: 76 - protein, 113 aa, synthetic construct
  REGION 1..113: note = Antibody light Chain amino acid sequence
DIVMTQSTSSLTVTAGEKVTMSCKSSQSLLNSGNQKNYLTWYQQKPGQPPKLLIYWASSRESGVPDRFTGRGSGTDFTLTISSVQAEDLAVYYCQNDYSYPFTFGSGTKLEIK

SEQ ID NO: 77 - protein, 112 aa, synthetic construct
  REGION 1..112: note = Antibody light Chain amino acid sequence
DVLMTQSPLSLPVNLGDQASISCKSSQNIVHSNGNTYLEWYLQRPGQSPKLLIYKVSNRFSGVPDRFSGSGSGTDFTLKISRVEAEDLGVYYCFQGSHVPYTFGGGTKLEIK

SEQ ID NO: 78 - protein, 8 aa, synthetic construct
  REGION 1..8: note = Antibody Complementarity-determining regions (CDRs)
GYTFTDYY

SEQ ID NO: 79 - protein, 8 aa, synthetic construct
  REGION 1..8: note = Antibody Complementarity-determining regions (CDRs)
INPYTGAT

SEQ ID NO: 80 - protein, 14 aa, synthetic construct
  REGION 1..14: note = Antibody Complementarity-determining regions (CDRs)
ARGTYYSNYEGFPY

SEQ ID NO: 81 - protein, 6 aa, synthetic construct
  REGION 1..6: note = Antibody Complementarity-determining regions (CDRs)
QEISGY

SEQ ID NO: 82 - skipped

SEQ ID NO: 83 - protein, 9 aa, synthetic construct
  REGION 1..9: note = Antibody Complementarity-determining regions (CDRs)
LQYASYPYT

SEQ ID NO: 84 - protein, 8 aa, synthetic construct
  REGION 1..8: note = Antibody Complementarity-determining regions (CDRs)
GFTFTDYF

SEQ ID NO: 85 - protein, 8 aa, synthetic construct
  REGION 1..8: note = Antibody Complementarity-determining regions (CDRs)
INPNNGGT

SEQ ID NO: 86 - protein, 15 aa, synthetic construct
  REGION 1..15: note = Antibody Complementarity-determining regions (CDRs)
VGEGTYYGRGWYFDV

SEQ ID NO: 87 - protein, 10 aa, synthetic construct
  REGION 1..10: note = Antibody Complementarity-determining regions (CDRs)
KSVSTSGYSY

SEQ ID NO: 88 - skipped

SEQ ID NO: 89 - protein, 8 aa, synthetic construct
  REGION 1..8: note = Antibody Complementarity-determining regions (CDRs)
QHIRELYT

SEQ ID NO: 90 - protein, 8 aa, synthetic construct
  REGION 1..8: note = Antibody Complementarity-determining regions (CDRs)
GYTFTDYY

SEQ ID NO: 91 - protein, 8 aa, synthetic construct
  REGION 1..8: note = Antibody Complementarity-determining regions (CDRs)
IFPGTGST

SEQ ID NO: 92 - protein, 16 aa, synthetic construct
  REGION 1..16: note = Antibody Complementarity-determining regions (CDRs)
ASPIYYGNHYEGYFDY

SEQ ID NO: 93 - protein, 10 aa, synthetic construct
  REGION 1..10: note = Antibody Complementarity-determining regions (CDRs)
KSVSTSGYSY

SEQ ID NO: 94 - skipped

SEQ ID NO: 95 - protein, 8 aa, synthetic construct
  REGION 1..8: note = Antibody Complementarity-determining regions (CDRs)
QHIRELYT

SEQ ID NO: 96 - protein, 8 aa, synthetic construct
  REGION 1..8: note = Antibody Complementarity-determining regions (CDRs)
GYSFTGYY

SEQ ID NO: 97 - protein, 8 aa, synthetic construct
  REGION 1..8: note = Antibody Complementarity-determining regions (CDRs)
INPSTGTT

SEQ ID NO: 98 - protein, 14 aa, synthetic construct
  REGION 1..14: note = Antibody Complementarity-determining regions (CDRs)
ARGGYYYGSSYVGY

SEQ ID NO: 99 - protein, 10 aa, synthetic construct
  REGION 1..10: note = Antibody Complementarity-determining regions (CDRs)
KSVSTSGYSY

SEQ ID NO: 100 - skipped

SEQ ID NO: 101 - protein, 8 aa, synthetic construct
  REGION 1..8: note = Antibody Complementarity-determining regions (CDRs)
QHIRELYT

SEQ ID NO: 102 - protein, 8 aa, synthetic construct
  REGION 1..8: note = Antibody Complementarity-determining regions (CDRs)
GYIFSYSW

SEQ ID NO: 103 - protein, 8 aa, synthetic construct
  REGION 1..8: note = Antibody Complementarity-determining regions (CDRs)
IYPGDGDD

SEQ ID NO: 104 - protein, 14 aa, synthetic construct
  REGION 1..14: note = Antibody Complementarity-determining regions (CDRs)
ARLHIYDGYYVSVY

SEQ ID NO: 105 - protein, 10 aa, synthetic construct
  REGION 1..10: note = Antibody Complementarity-determining regions (CDRs)
KSVSTSGYSY

SEQ ID NO: 106 - skipped

SEQ ID NO: 107 - protein, 8 aa, synthetic construct
  REGION 1..8: note = Antibody Complementarity-determining regions (CDRs)
QHIRELYT

SEQ ID NO: 108 - protein, 8 aa, synthetic construct
  REGION 1..8: note = Antibody Complementarity-determining regions (CDRs)
GYTFTNFW

SEQ ID NO: 109 - protein, 8 aa, synthetic construct
  REGION 1..8: note = Antibody Complementarity-determining regions (CDRs)
IYPGTGNT

SEQ ID NO: 110 - protein, 13 aa, synthetic construct
  REGION 1..13: note = Antibody Complementarity-determining regions (CDRs)
AREAYYTNPGFAY

SEQ ID NO: 111 - protein, 11 aa, synthetic construct
  REGION 1..11: note = Antibody Complementarity-determining regions (CDRs)
QTIVHSNGNTY

SEQ ID NO: 112 - skipped

SEQ ID NO: 113 - protein, 9 aa, synthetic construct
  REGION 1..9: note = Antibody Complementarity-determining regions (CDRs)
FQGSHVPYT

SEQ ID NO: 114 - protein, 8 aa, synthetic construct
  REGION 1..8: note = Antibody Complementarity-determining regions (CDRs)
GYTFTDYN

SEQ ID NO: 115 - protein, 8 aa, synthetic construct
  REGION 1..8: note = Antibody Complementarity-determining regions (CDRs)
IYPNNGGT

SEQ ID NO: 116 - protein, 9 aa, synthetic construct
  REGION 1..9: note = Antibody Complementarity-determining regions (CDRs)
ARSGRGFAY

SEQ ID NO: 117 - protein, 6 aa, synthetic construct
  REGION 1..6: note = Antibody Complementarity-determining regions (CDRs)
QSISDY

SEQ ID NO: 118 - skipped

SEQ ID NO: 119 - protein, 9 aa, synthetic construct
  REGION 1..9: note = Antibody Complementarity-determining regions (CDRs)
QNGHSFPYT

SEQ ID NO: 120 - protein, 8 aa, synthetic construct
  REGION 1..8: note = Antibody Complementarity-determining regions (CDRs)
GYTFTAYN

SEQ ID NO: 121 - protein, 8 aa, synthetic construct
  REGION 1..8: note = Antibody Complementarity-determining regions (CDRs)
IRPTNGDT

SEQ ID NO: 122 - protein, 11 aa, synthetic construct
  REGION 1..11: note = Antibody Complementarity-determining regions (CDRs)
TRSTTVASFDY

SEQ ID NO: 123 - protein, 12 aa, synthetic construct
  REGION 1..12: note = Antibody Complementarity-determining regions (CDRs)
QSLLYTNNQKNF

SEQ ID NO: 124 - skipped

SEQ ID NO: 125 - protein, 9 aa, synthetic construct
  REGION 1..9: note = Antibody Complementarity-determining regions (CDRs)
QQYYDFPPT

SEQ ID NO: 126 - protein, 8 aa, synthetic construct
  REGION 1..8: note = Antibody Complementarity-determining regions (CDRs)
GYTFTNFW

SEQ ID NO: 127 - protein, 8 aa, synthetic construct
  REGION 1..8: note = Antibody Complementarity-determining regions (CDRs)
IYPGTGNT

SEQ ID NO: 128 - protein, 13 aa, synthetic construct
  REGION 1..13: note = Antibody Complementarity-determining regions (CDRs)
AREAYYTNPGFAY

SEQ ID NO: 129 - protein, 10 aa, synthetic construct
  REGION 1..10: note = Antibody Complementarity-determining regions (CDRs)
KSVSTSGYSY

SEQ ID NO: 130 - skipped

SEQ ID NO: 131 - protein, 8 aa, synthetic construct
  REGION 1..8: note = Antibody Complementarity-determining regions (CDRs)
QHIRELYT

SEQ ID NO: 132 - protein, 8 aa, synthetic construct
  REGION 1..8: note = Antibody Complementarity-determining regions (CDRs)
GYIFSYSW

SEQ ID NO: 133 - protein, 8 aa, synthetic construct
  REGION 1..8: note = Antibody Complementarity-determining regions (CDRs)
IYPGDGDA

SEQ ID NO: 134 - protein, 14 aa, synthetic construct
  REGION 1..14: note = Antibody Complementarity-determining regions (CDRs)
ARLHIYDGYYVSVY

SEQ ID NO: 135 - protein, 6 aa, synthetic construct
  REGION 1..6: note = Antibody Complementarity-determining regions (CDRs)
QDINNY

SEQ ID NO: 136 - skipped

SEQ ID NO: 137 - protein, 9 aa, synthetic construct
  REGION 1..9: note = Antibody Complementarity-determining regions (CDRs)
QQGDTLPWT

SEQ ID NO: 138 - protein, 8 aa, synthetic construct
  REGION 1..8: note = Antibody Complementarity-determining regions (CDRs)
GYTFTNFW

SEQ ID NO: 139 - protein, 8 aa, synthetic construct
  REGION 1..8: note = Antibody Complementarity-determining regions (CDRs)
IYPGSGNT

SEQ ID NO: 140 - protein, 13 aa, synthetic construct
  REGION 1..13: note = Antibody Complementarity-determining regions (CDRs)
AREAYYTNPGFAY

SEQ ID NO: 141 - protein, 11 aa, synthetic construct
  REGION 1..11: note = Antibody Complementarity-determining regions (CDRs)
QNIVHSNGNTY

SEQ ID NO: 142 - skipped

SEQ ID NO: 143 - protein, 9 aa, synthetic construct
  REGION 1..9: note = Antibody Complementarity-determining regions (CDRs)
FQGSHVPYT

SEQ ID NO: 144 - protein, 8 aa, synthetic construct
  REGION 1..8: note = Antibody Complementarity-determining regions (CDRs)
GYTFTNFW

SEQ ID NO: 145 - protein, 8 aa, synthetic construct
  REGION 1..8: note = Antibody Complementarity-determining regions (CDRs)
IYPGSGNT

SEQ ID NO: 146 - protein, 13 aa, synthetic construct
  REGION 1..13: note = Antibody Complementarity-determining regions (CDRs)
AREAYYTNPGFAY

SEQ ID NO: 147 - protein, 10 aa, synthetic construct
  REGION 1..10: note = Antibody Complementarity-determining regions (CDRs)
KSVSTSGYSY

SEQ ID NO: 148 - skipped

SEQ ID NO: 149 - protein, 8 aa, synthetic construct
  REGION 1..8: note = Antibody Complementarity-determining regions (CDRs)
QHIRELYT

SEQ ID NO: 150 - protein, 8 aa, synthetic construct
  REGION 1..8: note = Antibody Complementarity-determining regions (CDRs)
GYTFTDYY

SEQ ID NO: 151 - protein, 8 aa, synthetic construct
  REGION 1..8: note = Antibody Complementarity-determining regions (CDRs)
INPYTGAT

SEQ ID NO: 152 - protein, 14 aa, synthetic construct
  REGION 1..14: note = Antibody Complementarity-determining regions (CDRs)
ARGTYYSNYEGFPY

SEQ ID NO: 153 - protein, 6 aa, synthetic construct
  REGION 1..6: note = Antibody Complementarity-determining regions (CDRs)
QEISGY

SEQ ID NO: 154 - skipped

SEQ ID NO: 155 - protein, 9 aa, synthetic construct
  REGION 1..9: note = Antibody Complementarity-determining regions (CDRs)
LQYASYPYT

SEQ ID NO: 156 - protein, 8 aa, synthetic construct
  REGION 1..8: note = Antibody Complementarity-determining regions (CDRs)
GYTFTDYY

SEQ ID NO: 157 - protein, 8 aa, synthetic construct
  REGION 1..8: note = Antibody Complementarity-determining regions (CDRs)
INPYTGAT

SEQ ID NO: 158 - protein, 14 aa, synthetic construct
  REGION 1..14: note = Antibody Complementarity-determining regions (CDRs)
ARGTYYSNYEGFPY

SEQ ID NO: 159 - protein, 6 aa, synthetic construct
  REGION 1..6: note = Antibody Complementarity-determining regions (CDRs)
QEISGY

SEQ ID NO: 160 - skipped

SEQ ID NO: 161 - protein, 9 aa, synthetic construct
  REGION 1..9: note = Antibody Complementarity-determining regions (CDRs)
LQYASYPYT

SEQ ID NO: 162 - protein, 8 aa, synthetic construct
  REGION 1..8: note = Antibody Complementarity-determining regions (CDRs)
GYTFTDYY

SEQ ID NO: 163 - protein, 8 aa, synthetic construct
  REGION 1..8: note = Antibody Complementarity-determining regions (CDRs)
IFPGTGST

SEQ ID NO: 164 - protein, 16 aa, synthetic construct
  REGION 1..16: note = Antibody Complementarity-determining regions (CDRs)
ASPIYYGNHYEGYFDY

SEQ ID NO: 165 - protein, 10 aa, synthetic construct
  REGION 1..10: note = Antibody Complementarity-determining regions (CDRs)
KSVSTSGYSY

SEQ ID NO: 166 - skipped

SEQ ID NO: 167 - protein, 8 aa, synthetic construct
  REGION 1..8: note = Antibody Complementarity-determining regions (CDRs)
QHIRELYT

SEQ ID NO: 168 - protein, 8 aa, synthetic construct
  REGION 1..8: note = Antibody Complementarity-determining regions (CDRs)
GYSFTSFF

SEQ ID NO: 169 - protein, 8 aa, synthetic construct
  REGION 1..8: note = Antibody Complementarity-determining regions (CDRs)
IHPGSGNT

SEQ ID NO: 170 - protein, 12 aa, synthetic construct
  REGION 1..12: note = Antibody Complementarity-determining regions (CDRs)
AREGFQYDYFPY

SEQ ID NO: 171 - protein, 11 aa, synthetic construct
  REGION 1..11: note = Antibody Complementarity-determining regions (CDRs)
QPIVHSNGNTY

SEQ ID NO: 172 - skipped

SEQ ID NO: 173 - protein, 9 aa, synthetic construct
  REGION 1..9: note = Antibody Complementarity-determining regions (CDRs)
FQGMQVAYT

SEQ ID NO: 174 - protein, 8 aa, synthetic construct
  REGION 1..8: note = Antibody Complementarity-determining regions (CDRs)
GYTFTDYN

SEQ ID NO: 175 - protein, 8 aa, synthetic construct
  REGION 1..8: note = Antibody Complementarity-determining regions (CDRs)
IYPNNGGT

SEQ ID NO: 176 - protein, 9 aa, synthetic construct
  REGION 1..9: note = Antibody Complementarity-determining regions (CDRs)
ARSGRGFAY

SEQ ID NO: 177 - protein, 12 aa, synthetic construct
  REGION 1..12: note = Antibody Complementarity-determining regions (CDRs)
QSLLNSGNQKNY

SEQ ID NO: 178 - skipped

SEQ ID NO: 179 - protein, 9 aa, synthetic construct
  REGION 1..9: note = Antibody Complementarity-determining regions (CDRs)
QNDYSYPFT

SEQ ID NO: 180 - protein, 8 aa, synthetic construct
  REGION 1..8: note = Antibody Complementarity-determining regions (CDRs)
GYTFTNFW

SEQ ID NO: 181 - protein, 8 aa, synthetic construct
  REGION 1..8: note = Antibody Complementarity-determining regions (CDRs)
IYPGTGNT

SEQ ID NO: 182 - protein, 13 aa, synthetic construct
  REGION 1..13: note = Antibody Complementarity-determining regions (CDRs)
AREAYYTNPGFAY

SEQ ID NO: 183 - protein, 11 aa, synthetic construct
  REGION 1..11: note = Antibody Complementarity-determining regions (CDRs)
QNIVHSNGNTY

SEQ ID NO: 184 - skipped

SEQ ID NO: 185 - protein, 9 aa, synthetic construct
  REGION 1..9: note = Antibody Complementarity-determining regions (CDRs)
FQGSHVPYT

SEQ ID NO: 186 - protein, 357 aa, synthetic construct
  REGION 1..357: note = Antibody heavy Chain nucleic acid sequence
CAGGTTCAGCTGCAGGAGTCTGGAGCTGAGCTGATGAAGCCTGGGGCCTCAGTGAAGATATCCTGCAAGGCTACTGGCTACACATTCAGTAGCTACTGGATAGAGTGGGTAAAGCAGAGGCCTGGACATGGCCTTGAGTGGATTGGAGAGATTTTACCTGGAAGTGGTAGTACTAAATATAATGAGAAGTTCAAGGGCAAGGCCACATTCACTGCAGATCCATCCTCCAACACAGCCTACATGCAACTCAGCAGCCTGACATCTGATGACTCTGCCGTCTATTACTGTACTTCTTACTTCGGTAGTAGCTCCTCGTTTGCTTACTGGGGCCAAGGGACTCTGGTCACTGTCTCTGCA

SEQ ID NO: 187 - protein, 357 aa, synthetic construct
  REGION 1..357: note = Antibody heavy Chain nucleic acid sequence
CAGGTTCAGCTGCAGGAGTCTGACGCTGTGTTGGTGAAACCTGGGGCTTCAGTGAAGATATCCTGCAAGGCTTCTGGCTACACCTTCATTGACCATGGTCTTCACTGGGTGAAGCAGAAGCCTGAACAGGGCCTGGAATGGATTGGATATATTTCTCCCGGAAATGGTGATATCAAGTACAATGAGAAGTTCAAGGGCAAGGCCACACTGACTGCAGACAAATCCTCCAGCACTGCCTACATGCAGCTCAACAGCCTGACATCTGAGGATTCTGCAGTGTATTTCTGTAAAAGGGGGGTTCGACGAGGGGGCTATGCTATGGACTACTGGGGTCAAGGAACCTCAGTCACCGTCTCC

SEQ ID NO: 188 - protein, 339 aa, synthetic construct
  REGION 1..339: note = Antibody heavy Chain nucleic acid sequence
CAGGTCCAACTGCAGCAGCCTGGGACTGAACTGGTGAAGCCTGGGGCTTCAGTGAAGCTGTCCTGCAAGGCTTCTGGCTACACCTTCACCAGCTACTGGATGCACTGGGTGAAACAGAGGCCTGGACTAGGCCTTGAGTGGATTGGGGGGATTAATCCTAGCAATGGTGCTACTGACTTCAATGAGAGGTTCAAGAACAAGGCCACACTGACTGTTGACATATCTTCCACCACGGCCTACATACAGTTCAGTAGCCTGACATCTGAGGACTCTGCGGTCTATTTCTGTACAAGAGGGAGTCCTTACTGGGGCCAGGGGACTCTGGTCACTGTCTCTGCA

SEQ ID NO: 189 - protein, 363 aa, synthetic construct
  REGION 1..363: note = Antibody heavy Chain nucleic acid sequence
CAGGTCCAACTGCAGGAGTCTGGGGCTGAGCTGGTGAAGCCTGGGACTTCAGTGAAGATGTCCTGCAAGGCTTCTGGTTACACCTTCACCACCTACTGGATGCACTGGGTGAAGCAGAGGCCTGGACAAGGCCTTGAGTGGATCGGAACGATTGATCCTTTAGATAGTTATGCTACCTACAATCAGAATTTCACGGGCAAGACCACATTGACTGTAGACACATCCTCCAGCACAGCCTACATGCAGCTCAACAGCCTGACATCTGAGGACTCTGCGGTCTATTATTGTACAAGAGGTGGAGATGGTGACTACTACTTTTCTCTGGACTACTGGGGTCAAGGAACCTCACTCACCGTCTCCTCA

SEQ ID NO: 190 - protein, 354 aa, synthetic construct
  REGION 1..354: note = Antibody heavy Chain nucleic acid sequence
GAGTTCCAGCTGCAGGAGTCTGGACCTGACCTGGAGAAGCCTGGCGCTTCAGTGAAGATATCCTGCAAGGCTTCTGGTTACTCATTCACTGGCTACAACTTGAACTGGGTGAAACAGAGCAATGGAAAGAGTCTTGACTGGATTGGAAATATTGATCCTTACTATGGTGGTGCTGACTACAACCAGAAGTTCAAGGGCAAGGCCACATTGACTGTAGACAAGTCTTCCAGCACAGCCTACATGCAGCTCAAGAGCCTGACATCTGAGGACTCTGCAGTCTATTACTGTGCAAGAGGGGATGATTACGTCGACTTTGCTTACTGGGGCCAAGGGACTCTGGTCACTGTCTCTGCA

SEQ ID NO: 191 - protein, 357 aa, synthetic construct
  REGION 1..357: note = Antibody heavy Chain nucleic acid sequence
CAGGTGCAGCTGAAGGAGTCAGGACCTGGCCTGGTGGCGCCCTCACAGAGCCTGTCCATCACATGCACTGTTTCAGGGTTCTCATTAACCGACTATGGTGTAAACTGGATTCGACAGTCTCCAGGAAAGGGTCTGGAGTGGCTGGGAGTAATATGGGGTGGTGGCAGTACATACTATAATTCAGGTCTCAAATCCAGACTGAGCATCAGTAAGGACAACTCCAAGAGCCAAGTTTTCTTAAAAATGAACAGTCTGCAAACTGATGACACAGCCATGTACTACTGCGCCAAACATTACCCTGGCAACTACTCTGCTATGGACTACTGGGGTCAAGGAACCTCAGTCACCGTCTCCTCA

SEQ ID NO: 192 - protein, 360 aa, synthetic construct
  REGION 1..360: note = Antibody heavy Chain nucleic acid sequence
GAGTTCCAGCTGCAGCAGTCTGGACCTGAACTGGTTAAGCCTGGGGCTTCACTGAGGATGTCCTGCAAGGCTTCTGGATACACATTCAGTCTCTATATTATGCACTGGGTGAAACAGAAGCCTGAGCAGGGCCTTGAGTGGATTGGATATTTTAATCCTGACAATGATGATACTAAATACAATGACAAATTCAAAGGCAAGGCCACACTGACTTCAGACAAATCCTCCAGCACAGCCTACATGGAACTCACCAGCCTGACCTCTGAGGACTCTGCGGTCTACTATTGTACTAGAGAGGATTACTATGGCTTCTGGTACTTCGATGTCTGGGGCGCAGGGACCACGGTCACCGTCTCCTCA

SEQ ID NO: 193 - protein, 357 aa, synthetic construct
  REGION 1..357: note = Antibody heavy Chain nucleic acid sequence
GAGGTGCAGCTTCAGGAGTCAGGACCTGGCCTGGCAAAACCTTCTCAGACTCTGTCCCTCACCTGTTCTGTCACTGGCGACTCCATCACCAGTGGTTGCTGGAACTGGATCCGGAAATTCCCAGGGAATAAACTTGAGTACATGGGGTACATAAGCTACAGTGGTATCACTTACTACAGTCCATCTCTCAAAAGTCGAATCTCCATCACTCGAGACACGTCCAAGAACCAGTACTACCTACTGTTGAATTCTGTGACTACTGAGGACTCAGCCACATATTACTGTTCAAGATCATCTACTATGATTACGACGGGGTTTGCTTACTGGGGCCAAGGGACTCTGGTCACTGTCTCTGCA

SEQ ID NO: 194 - protein, 351 aa, synthetic construct
  REGION 1..351: note = Antibody heavy Chain nucleic acid sequence
CAGATCCAGTTGGTACAGTCTGGACCTGAGCTGAAGAAGCCTGGAGAGACAGTCAAGATCTCCTGCAAGGCTTCTGGGTATACCTTCACAACCTATGGAATGAGCTGGGTGAAACAGGCTCCAGGAAAGGGTTTAAAGTGGATGGGCTGGATAAACACCTACTCTGGAGTGCCAACATATGCTGATGACTTCAAGGGACGGTTTGCCTTCTCTTTGGAAACCTCTGCCAGTACTGCCTATTTGCAGATCAACACCCTCAAAAGTGAGGACACGGCCACTTACTTCTGTACAAGATCTTATAGTTATTACTTTGACATCTCGGGCCAAGGCACCACTACACACAGTCTCCTC

SEQ ID NO: 195 - protein, 363 aa, synthetic construct
  REGION 1..363: note = Antibody heavy Chain nucleic acid sequence
CAGGTGCAGCTGAAGGAGTCAGGACCTGGCCTGGTGGCGCCCTCACAGAGCCTGTCCATCACATGCACCGTCTCAGGGTTCTCATTAACCGGCTATGGTGTAAACTGGGTTCGCCAGCCTCCAGGAAAGGGTCTGGAGTGGCTGGGAATGATATGGGGTGATGGAAGCACAGACTATAATTCAGCTCTCAAATCCAGACTGAGCATCAGCAAGGACAACTCCAAGAGCCAAGTTTTCTTAAAAATGAACAGTCTGCAAACTGATGACACAGCCAGGTATTACTGTGCCAGAGGATGGGAAAGTCTATCTACTATGTCCTGGTTTGCTTACTGGGGCCAAGGGACTCTGGTCACTGTCTCTGCA

SEQ ID NO: 196 - protein, 360 aa, synthetic construct
  REGION 1..360: note = Antibody heavy Chain nucleic acid sequence
GAGGTGCAGCTGCAGGAGTCTGGACCTGAACTGGTTAAGCCTGGGGCTTCACTGAGGATGTCCTGCAAGGCTTCTGGATACACATTCACTCTCTATATTATGCACTGGGTGAAACAGAAGCCTGGGCAGGGCCTTGAGTGGATTGGATATTTTAATCCTGACAATGATGATACTAAATACAATGACAAATTCAAGGGCAAGGCCACACTGACTTCAGACAAATCCTCCAACACAGCCTACATGGAACTCAGCAGCCTGACCTCTGAGGACTCTGCGGTCTATTACTGTGCAAGAGAGGATTACTATGGCTTCTGGTACTTCGATGTCTGGGGCGCAGGGACCACGGTCACCGTCTCCTCA

SEQ ID NO: 197 - protein, 363 aa, synthetic construct
  REGION 1..363: note = Antibody heavy Chain nucleic acid sequence
GATGTGCAGCTGGTGGAGTCTGGGGGAGGCTTAGTGCAGCCTGGAGGGTCCCGGAAACTCTCCTGTGCAGCCTCTGGATTCACTTTCAGTGGTTTCGGAATGCAGTGGGTTCGTCAGGCTCCAGAGAAGGGGCTGGAGTGGGTCGCATATATCAGTAGTGGCAGTAATATCATCCACTATAAAGACACAGTGAGGGGCCGATTCACCATCTCTAGAGACAATCCCAAGAACACCCTGTTCCTGCAAATGACCAGTCTAAGGTCTGAGGACACGGCCATGTATTACTGTGCAAGATCGGATAGGTATGACGTTTACTATGATATGGACTACTGGGGGCAAGGAACTTCAGTAACCGTCTCCTCA

SEQ ID NO: 198 - protein, 351 aa, synthetic construct
  REGION 1..351: note = Antibody heavy Chain nucleic acid sequence
CAGGTTCAGCTGCAGCAGTCTGGACCTGAGCTGGTGAAGCCTGGGGCCTCAGTGAAGATTTCCTGCAAAGCTCCTGGCTACGCATTCAGTACCTCTTGGATTAACTGGATGAGGCAGAGGCCTGGACAGGGTCTTGAATGGATTGGACGGATTTATCCTGGAGATGGAGATGATCACTACAATGGGAAGTTCAACGGCAAGGCCACTCTGACTGCAGACAAATCCTCCAGCACAGCCTACGTGCAGCTCAGCAGCCTGACCTCTGTGGACTCTGCGGTCTATTTCTGTGCAAGTTATGATGGTTACTCCTTTGCTTACTGGGGCCAAGGGACTTTGGTCACTGTCTCTGCA

SEQ ID NO: 199 - protein, 363 aa, synthetic construct
  REGION 1..363: note = Antibody heavy Chain nucleic acid sequence
GAGGTGCAGCTGCAGGAGTCTGGGGGAGGCTTGGTGCAGCCTGGAGGGTCCCGGAAACTCTCCTGTGCTACCTCTGGATTCACTTTCAGTACCTTTGGAATGCACTGGGTTCGTCAGGCTCCAGAGAAGGGGCTGGAGTGGGTCGCATACATTAGTAGTGGCAGTAGTATCATCTACTACGGAGACACAGTGAGGGGCCGATTCACCATCTCCAGAGACAATCCCAAGAACACCCTGCTCCTGCAAATGACCAGTCTAAGGTCTGAGGACACGGCCATGTATTACTGTGCAAGATCGGATAGGTACGACGTTTACTATAATATGGACTACTGGGGTCAAGGAACCTCAGTCACCGTCTCCTCA

SEQ ID NO: 200 - protein, 363 aa, synthetic construct
  REGION 1..363: note = Antibody heavy Chain nucleic acid sequence
GAGGTGCAGCTGCAGGAGTCTGGACCTAGCCTCGTGAAACCTTCTCAGACTCTGTCCCTCACCTGTTCTGTCACTGGCGACTCCATCACCAGTGGTTACTGGAACTGGATCCGGAAATTCCCAGGGAATCAAATTGAGTACATGGGGTACATAAGGTACAGTGGTAGCACTTACTACAATCCATCTCTCAAAAGTCGAATCTCCATCACTCGAGACACATCCAAGAACCAGTACTACCTGCAGTTGAATTCTGTGACTACTGCGGACACAGCCACATATTACTGTGCAAGATATGTAGTACGGCTACCTCATTACTATGCTATGGACTACTGGGGTCAAGGAACCTCAGTCACCGTCTCCTCA

SEQ ID NO: 201 - protein, 351 aa, synthetic construct
  REGION 1..351: note = Antibody heavy Chain nucleic acid sequence
CAGGTCCAGCTGCAGGAGTCTGGAGCTGAACTGGTAAGGCCTGGGACTTCAGTGAAGGTGTCCTGCAAGGCTTCTGGATACGCCTTCACTAATTACTTGATAGAGTGGCTGAAGCTGAGGCCTGGACAGGGCCTTGAGTGGATTGGAGTGATTAATCCTGGAAGTGATAGAACTAATTACAATGAGAAGTTCAAGGGCAAGGCAACATTTACTGCAGACAAATCCTCCAGCACTGCCTACATGCAGCTCAGCAGCCTGACATCTGAAGACTCTGCGGTCTACTTCTGTGCAACCGACAGCTCGGGCAATGGGGACTACTGGGGTCAAGGAACCTCAGTCACCGTCTCCTCA

SEQ ID NO: 202 - protein, 348 aa, synthetic construct
  REGION 1..348: note = Antibody heavy Chain nucleic acid sequence
CAGGTGCAGCTGCAGGAGTCTGGGGCTGAGCTGGTGAGGCCTGGGTCCTCAGTGAAGATTTCCTGCAAGGCTTCTGGCAATGCATTCAGTAGCTACTGGATGAACTGGGTGAAGCAGAGGCCTGGACAGGGTCTTGAGTGGATTGGACAGATTCATCCTGGAGATGATGATTCTAACTACAATGGAAAGTTCAAGGGTAAAGCCATACTGACTGCAGACAAATCCTCCAGCACAGCCTACATGCAGCTCAGCAGCCTAACATCTGAGGACTCTGCGGTCTATTTCTGTGCAAGAGGGGAGGTACGACTTGACTACTGGGGCCAAGGCACCACTCTCACAGTCTCCTCA

SEQ ID NO: 203 - protein, 369 aa, synthetic construct
  REGION 1..369: note = Antibody heavy Chain nucleic acid sequence
GAAGTGAAGCTTGAGGAGTCTGGAGGAGGCTTGGTGCAACCTGGAGGATCCATGAAACTCTCCTGTGTTGCCTCTGGATTCATTTTCAGTAACTACTGGATGAATTGGGTCCGCCAGTCTCCAGAGAAGGGGCTTGAGTGGGTTGCTGAAATCAGAGTGAAATCTAAGAACTTTGCAACACATTATGCGGAGTCTGTGAAAGGGACGTTCACCGTCTCAAGAGATGATTCCAAAAGTACTGTCTACCTGCAAATGAACAACCTAAGAGCTGAAGACACTGGCATTTATTACTGTACCAGGACCTACTATAGTAACTACAACTGGTACTTCGATGTCTGGGGCGCTGGGACCACGGTCACCGTCTCCTCA

SEQ ID NO: 204 - protein, 363 aa, synthetic construct
  REGION 1..363: note = Antibody heavy Chain nucleic acid sequence
GATGTGCAGCTGGTGGAGTCTGGGGGAGGCTTAGTGCAGCCTGGAGGGTCCCGGAAACTCTCCTGTGTAGCCTCTGGATTCTCTTTCAGTAGTTTTGGAATGCACTGGGTTCGTCAGGCTCCAGAAAAGGGGCTGGAGTGGGTCGCATATATTAGTAGTGGCAGTCGTATTATCTATTATGAAGACAAAGTCAAGGGCCGATTCACCATTTCTAGAGACAATCCCAAGAACACCCTGTTCCTGCAAATGACCAGTCTAAGGTCTGAGGACACGGCCATGTATTACTGTGCAAGATCGGATAGGTACGACGTTTATTATTCTATAGACTACTGGGGTCAAGGAACCTCAGTCACCGTCTCCTCA

SEQ ID NO: 205 - protein, 363 aa, synthetic construct
  REGION 1..363: note = Antibody heavy Chain nucleic acid sequence
GATGTGCAGCTGGTGGAGTCTGGGGGAGGCTTAGTGCAGCCTGGAGGGTCCCGGAAACTCTCCTGTGCAGCCTCTGGATTCACTTTCAGTACCTTTGGCATGCACTGGATTCGTCAGGCTCCAGAGAAGGGGCTGGAGTGGGTCGCATATATTAGTAGTGACAGTCGTATCATCTACTATGCCGACACAGTGAGGGGCCGATTCTCCATCTCCAGAGACAATCCCAAGAACACCCTGTTCCTGCAAATGACCACTCTAAAGTCTGAGGACACGGCCATGTATTACTGTGCAAGATCGGATAGATACGACGTTTACTATTCTATGAACTACTGGGGTCAAGGAACCTCAGTCACCGTCTCCTCA

SEQ ID NO: 206 - protein, 351 aa, synthetic construct
  REGION 1..351: note = Antibody heavy Chain nucleic acid sequence
GAGGTGCAGCTGCAGGAGTCTGGACCTGAGCTGGTCAAGCCTGGGGCTTCAGTGAAGATGTCCTGCAAGGCTTCTGGATACACATTCACTAACTATGTTATGCACTGGGTGAAGCAGAAGCCTGGGCAGGGCCTTGAGTGGATTGGATACATTAATCCTGACAATGATGGTGCTAACTACAATGAGAAGTTCAAAAACAAGGCCACACTGACTTCGGACAAATCCTCCAACACAGCCTACATGGAGCTCAGCAGCCTGACCTCTGAGGACTCTGCGGTCTATTACTGTGCAAGAGCAGCTCGGGTCTGGTTTCCTTACTGGGGCCAAGGGACTCTGGTCACTGTCTCTGCA

SEQ ID NO: 207 - protein, 369 aa, synthetic construct
  REGION 1..369: note = Antibody heavy Chain nucleic acid sequence
GAGGTGCAGCTGCAGGAGTCTGGAGGAGGCTTGGTACAGCCTGGGGGTTCTCTGAGACTCTCCTGTGCAACTTCTGGGTTCACCTTCACTGATTACTACATGAGCTGGGTCCGCCAGCCTCCAGGAAAGGCACTTGAGTGGTTGGGTTTTATTAGAAACAAAGCTAATGGTTACACAACAGAATACAGTGCATCTGTGAAGGGTCGGTTCACCATCTCCAGAGATAATTCCCAAAGCATCCTCTATCTTCAAGTGAACACCCTGAGAGCTGAGGACAGTGCCACTTATTACTGTGCAAGAGATCCTCTCTATGGTAGGTCCTATGCTTTGGACTACTGGGGTCAAGGAACCTCAGTCACCGTCTCCTCA

SEQ ID NO: 208 - protein, 351 aa, synthetic construct
  REGION 1..351: note = Antibody heavy Chain nucleic acid sequence
GAGGTGCAGCGGCAGGAGTCTGGGGGAGGCTTAGTGAAGCCTGGAGGGTCCCTGAAACTCTCCTGTGCAGCCTCTGGATTCACTTTCAGTAGCTATGCCATGTCTTGGGTTCGCCAGTCTCCAGAGAAGAGTCTGGAGTGGGTCGCAGAAATTAGTAATGGTGGTAGTTACACCTACTATCCAGACACTGTGACGGGCCGATTCACCATATCCAGAGACAATGCCAAGAACACCCTGTACCTGGAAATGAGCAGTCTTAGGTCTGAAGACACGGCCATGTATTACTGTGCAAGGGATCGGAATGGTTACTTTGACTACTGGGGCCAAGGCACCAGTCTCACAGTCTCCTCA

SEQ ID NO: 209 - protein, 363 aa, synthetic construct
  REGION 1..363: note = Antibody heavy Chain nucleic acid sequence
GATGTGCAGCTGGTGGAGTCTGGGGGAGGCTTAGTGCGGCCTGGAGGGTCCCGGAGACTCTCCTGTGTAGTCTCTGGATTCACTTTCAGTAGTTTCGGAATGCACTGGGTTCGTCAGGCTCCAGAGAAGGGGCTGGAGTGGGTCGCATATATTAGTACTGGCAGTAGTATCATCTACTATACAGACACAGTGCGGGGCCGATTCACCATCTCCAGAGACAATCCCAAGAACACCCTGTTCCTGCAAATGACCAGTCTAAGGTCTGAGGATACGGCCATATATTACTGTGCAAGATCGGATAGGTACGACGTTTACTATGATATGGACTATTGGGGTCAAGGAACTTCAGTAACCGTCTCCTCA

SEQ ID NO: 210 - protein, 363 aa, synthetic construct
  REGION 1..363: note = Antibody heavy Chain nucleic acid sequence
GAGTTCCAGCTGCAGCAGTCTGGACCTGAGCTGGAGAAGCCTGGCGCTTCAGTGAAGATATCCTGCAGGGCTTCTGGTTACTCATTCACTGGCTACAACATGAACTGGGTGAAGCAGAACAATGGAAAGAGCCTTGAGTGGATTGGAAATATTGATCCTTACTATGGTGGTACTAGCTACAACCAGAAGTTCAAGGGCAAGGCCACATTGACTGTAGACAAATCGTCCAGCACAGCCTACATGCAGCTCAAGAGCCTGACATCTGAGGACTCTGCAGTCTATTACTGTGCAAGATACTATAGGTACGACGTAGGCGGGTACTTCGATGTCTGGGGCGCAGGGACCACGGTCACCGTCTCCTCA

SEQ ID NO: 211 - protein, 351 aa, synthetic construct
  REGION 1..351: note = Antibody heavy Chain nucleic acid sequence
GATGTGCAGCTGGTGGAGTCTGGGGGAGGCTTAGTGCAGCCTGGAGGGTCCCGGAAACTCTCCTGTGCAGCCTCTGGATTCACTTTCAGTAGCTTTGGAATGCACTGGGTTCGTCAGGCTCCAGAGAAGGGGCTGGAGTGGGTCGCATACATTAGTAGTGGCAATACTACCATCAACTATGCAGACACAGTGAAGGGCCGATTCACCATTTCCAGAGACAATCCCAAGAACACCCTGTTTCTGCAAATGACCAGTCTAAGGTCTGAGGACACGGCCATGTATTACTGTGCAAGATACTTTTTCTATGCTATGGACTACTGGGGTCAAGGAACCTCAGTCACCGTCTCCTCA

SEQ ID NO: 212 - protein, 363 aa, synthetic construct
  REGION 1..363: note = Antibody heavy Chain nucleic acid sequence
GATGTGCAGCTGGTGGAGTCTGGGGGAGGCTTAGTGCAGCCTGGAGGGTCCCGGAAACTCTCCTGTGCAGCCTCTGGATTCACTTTCAGTACCTTTGGAATGCACTGGGTTCGTCAGGCTCCAGAGAAGGGGCTGGAGTGGGTCGCATACATTAGTCGTGGCAGTATTATCATCCACTATGGAGACACAGTGAAGGGCCGATTCACCATCTCCAGAGACAATCCCAAGAACACCCTGTTCCTGCAAATGACCAGTCTAAGGTCTGAGGACACGGCCATGTATTACTGTGTAAGATCGGATAGGTACGACGTTTACTATGACATGGACTACTGGGGTCAAGGAACGTCAGTCACCGTCTCCTCA

SEQ ID NO: 213 - protein, 363 aa, synthetic construct
  REGION 1..363: note = Antibody heavy Chain nucleic acid sequence
GATGTGCAGCTGGTGGAGTCTGGGGGAGGCTTAGTGCAGCCTGGAGGGTCCCGGAAACTCTCCTGTGTAGTCTCTGGATTCACTTTCAGTGGTTTCGGAATGCACTGGGTTCGTCAGGCTCCAGAAAAGGGGCTGGAGTGGGTCGCATATATTAGTACTGGCAGTACTATCATCTACTATACAGACACAGTGCGGGGCCGATTCACCATCTCCAGAGACAATCCCAAGAACACCCTGTTCCTGCAAATGACCAGTCTAAGGTCTGAGGACACGGCCATGTATTACTGTGCAAGATCGGATAGGTACGACGTTTACTATGATATGGACTACTGGGGTCAAGGAACTTCAGTAACCGTCTCCTCA

SEQ ID NO: 214 - protein, 357 aa, synthetic construct
  REGION 1..357: note = Antibody heavy Chain nucleic acid sequence
GAAGTGAAGCTTGAGGAGTCTGGAGGAGGCTTGGTGCAACCTGGAGGATCCATGAAAATCTCTTGTACTGCCTCTGGATTCACTTTTACTGACGCCTGGATGGACTGGGTCCGCCAGTCTCCAGAGAAGGGGCTTGAGTGGGTTGCTGAAGTTAGAAGCAAAGCTAAAAATTATGCAACATATTATCCTGAGTCTGTGAAAGGGAGGTTCACCATCTCAAGAGATGATTCCAAAAGTAGTGTCTACCTTCAAATGAACAACTTAAGAGCTGAAGACACTGGCGTTTATTACTGTACCAGGAGTTACGTCTGGTACTTCGATGTCTGGGGCGCAGGGACCACGGTCACCGTCTCCTCA

SEQ ID NO: 215 - protein, 366 aa, synthetic construct
  REGION 1..366: note = Antibody heavy Chain nucleic acid sequence
CAGGTCCAGCTGCAGGAGTCTGGAGCTGAGCTGGTAAGGCCTGGGACTTCAGTGAAGGTGTCCTGCAAGGCTTCTGGATACGCCTTCACTAATTACTTGATAGAGTGGATAAAGCAGAGGCCTGGACAGGGCCTTGAGTGGATTGGAGTGATTAATCCTGGAAGTGGTGGTACTAACTACAATGAGAAGTTCAAGGGCAAGGCAACACTGACTGCAGACAAATCCTCCAGCACTGCCTACATGCAGCTCAGCAGCCTGACATCTGATGACTCTGCGGTCTATTTCTGTGCAAGATGGGATTTCGACGCGGGCGGTTTAAATTTAATTGCTTACTGGGGCCAAGGGACTCTGGTCACTGTCTCTGCA

SEQ ID NO: 216 - protein, 345 aa, synthetic construct
  REGION 1..345: note = Antibody heavy Chain nucleic acid sequence
CAGGTCCAACTGCAGGAGTCTGGGGCTGAGTTTGTGATGCCTGGGGCTCCAGTGAAGCTGTCCTGCAAGGCTTCTGGCTACACCTTCACCAACTACTGGATGAACTGGGTGAAACAGAGACCTGGACGAGGCCTCGAATGGATTGGGATGATTGATCCTTCCGATAGTGAAACTCACTACAATCAAAAGTTCAAGGACAAGGCCACACTGACTGTTGACAAATCTTCCAGCACAGCCTACATTCAACTCAGCAGCCTGACATCTGAGGACTCTGCGGTCTATTATTGTGCTTCATATGACTGGTTTGCTTATTGGGGCCAAGGGACTCTGGTCACTGTCTCTGCA

SEQ ID NO: 217 - protein, 119 aa, synthetic construct
  REGION 1..119: note = Antibody heavy Chain amino acid sequence
QVQLQESGAELMKPGASVKISCKATGYTFSSYWIEWVKQRPGHGLEWIGEILPGSGSTKYNEKFKGKATFTADPSSNTAYMQLSSLTSDDSAVYYCTSYFGSSSSFAYWGQGTLVTVSA

SEQ ID NO: 218 - protein, 119 aa, synthetic construct
  REGION 1..119: note = Antibody heavy Chain amino acid sequence
QVQLQESDAVLVKPGASVKISCKASGYTFIDHGLHWVKQKPEQGLEWIGYISPGNGDIKYNEKFKGKATLTADKSSSTAYMQLNSLTSEDSAVYFCKRGVRRGGYAMDYWGQGTSVTVS

SEQ ID NO: 219 - protein, 113 aa, synthetic construct
  REGION 1..113: note = Antibody heavy Chain amino acid sequence
QVQLQQPGTELVKPGASVKLSCKASGYTFTSYWMHWVKQRPGLGLEWIGGINPSNGATDFNERFKNKATLTVDISSTTAYIQFSSLTSEDSAVYFCTRGSPYWGQGTLVTVSA

SEQ ID NO: 220 - protein, 121 aa, synthetic construct
  REGION 1..121: note = Antibody heavy Chain amino acid sequence
QVQLQESGAELVKPGTSVKMSCKASGYTFTTYWMHWVKQRPGQGLEWIGTIDPLDSYATYNQNFTGKTTLTVDTSSSTAYMQLNSLTSEDSAVYYCTRGGDGDYYFSLDYWGQGTSLTVSS

SEQ ID NO: 221 - protein, 118 aa, synthetic construct
  REGION 1..118: note = Antibody heavy Chain amino acid sequence
EFQLQESGPDLEKPGASVKISCKASGYSFTGYNLNWVKQSNGKSLDWIGNIDPYYGGADYNQKFKGKATLTVDKSSSTAYMQLKSLTSEDSAVYYCARGDDYVDFAYWGQGTLVTVSA

SEQ ID NO: 222 - protein, 119 aa, synthetic construct
  REGION 1..119: note = Antibody heavy Chain amino acid sequence
QVQLKESGPGLVAPSQSLSITCTVSGFSLTDYGVNWIRQSPGKGLEWLGVIWGGGSTYYNSGLKSRLSISKDNSKSQVFLKMNSLQTDDTAMYYCAKHYPGNYSAMDYWGQGTSVTVSS

SEQ ID NO: 223 - protein, 120 aa, synthetic construct
  REGION 1..120: note = Antibody heavy Chain amino acid sequence
EFQLQQSGPELVKPGASLRMSCKASGYTFSLYIMHWVKQKPEQGLEWIGYFNPDNDDTKYNDKFKGKATLTSDKSSSTAYMELTSLTSEDSAVYYCTREDYYGFWYFDVWGAGTTVTVSS

SEQ ID NO: 224 - protein, 119 aa, synthetic construct
  REGION 1..119: note = Antibody heavy Chain amino acid sequence
EVQLQESGPGLAKPSQTLSLTCSVTGDSITSGCWNWIRKFPGNKLEYMGYISYSGITYYSPSLKSRISITRDTSKNQYYLLLNSVTTEDSATYYCSRSSTMITTGFAYWGQGTLVTVSA

SEQ ID NO: 225 - protein, 117 aa, synthetic construct
  REGION 1..117: note = Antibody heavy Chain amino acid sequence
QIQLVQSGPELKKPGETVKISCKASGYTFTTYGMSWVKQAPGKGLKWMGWINTYSGVPTYADDFKGRFAFSLETSASTAYLQINTLKSEDTATYFCTRSYSYYFDISGQGTTTHSLL

SEQ ID NO: 226 - protein, 121 aa, synthetic construct
  REGION 1..121: note = Antibody heavy Chain amino acid sequence
QVQLKESGPGLVAPSQSLSITCTVSGFSLTGYGVNWVRQPPGKGLEWLGMIWGDGSTDYNSALKSRLSISKDNSKSQVFLKMNSLQTDDTARYYCARGWESLSTMSWFAYWGQGTLVTVSA

SEQ ID NO: 227 - protein, 120 aa, synthetic construct
  REGION 1..120: note = Antibody heavy Chain amino acid sequence
EVQLQESGPELVKPGASLRMSCKASGYTFTLYIMHWVKQKPGQGLEWIGYFNPDNDDTKYNDKFKGKATLTSDKSSNTAYMELSSLTSEDSAVYYCAREDYYGFWYFDVWGAGTTVTVSS

SEQ ID NO: 228 - protein, 121 aa, synthetic construct
  REGION 1..121: note = Antibody heavy Chain amino acid sequence
DVQLVESGGGLVQPGGSRKLSCAASGFTFSGFGMQWVRQAPEKGLEWVAYISSGSNIIHYKDTVRGRFTISRDNPKNTLFLQMTSLRSEDTAMYYCARSDRYDVYYDMDYWGQGTSVTVSS

SEQ ID NO: 229 - protein, 117 aa, synthetic construct
  REGION 1..117: note = Antibody heavy Chain amino acid sequence
QVQLQQSGPELVKPGASVKISCKAPGYAFSTSWINWMRQRPGQGLEWIGRIYPGDGDDHYNGKFNGKATLTADKSSSTAYVQLSSLTSVDSAVYFCASYDGYSFAYWGQGTLVTVSA

SEQ ID NO: 230 - protein, 121 aa, synthetic construct
  REGION 1..121: note = Antibody heavy Chain amino acid sequence
EVQLQESGGGLVQPGGSRKLSCATSGFTFSTFGMHWVRQAPEKGLEWVAYISSGSSIIYYGDTVRGRFTISRDNPKNTLLLQMTSLRSEDTAMYYCARSDRYDVYYNMDYWGQGTSVTVSS

SEQ ID NO: 231 - protein, 121 aa, synthetic construct
  REGION 1..121: note = Antibody heavy Chain amino acid sequence
EVQLQESGPSLVKPSQTLSLTCSVTGDSITSGYWNWIRKFPGNQIEYMGYIRYSGSTYYNPSLKSRISITRDTSKNQYYLQLNSVTTADTATYYCARYVVRLPHYYAMDYWGQGTSVTVSS

SEQ ID NO: 232 - protein, 117 aa, synthetic construct
  REGION 1..117: note = Antibody heavy Chain amino acid sequence
QVQLQESGAELVRPGTSVKVSCKASGYAFTNYLIEWLKLRPGQGLEWIGVINPGSDRTNYNEKFKGKATFTADKSSSTAYMQLSSLTSEDSAVYFCATDSSGNGDYWGQGTSVTVSS

SEQ ID NO: 233 - protein, 116 aa, synthetic construct
  REGION 1..116: note = Antibody heavy Chain amino acid sequence
QVQLQESGAELVRPGSSVKISCKASGNAFSSYWMNWVKQRPGQGLEWIGQIHPGDDDSNYNGKFKGKAILTADKSSSTAYMQLSSLTSEDSAVYFCARGEVRLDYWGQGTTLTVSS

SEQ ID NO: 234 - protein, 123 aa, synthetic construct
  REGION 1..123: note = Antibody heavy Chain amino acid sequence
EVKLEESGGGLVQPGGSMKLSCVASGFIFSNYWMNWVRQSPEKGLEWVAEIRVKSKNFATHYAESVKGTFTVSRDDSKSTVYLQMNNLRAEDTGIYYCTRTYYSNYNWYFDVWGAGTTVTVSS

SEQ ID NO: 235 - protein, 121 aa, synthetic construct
  REGION 1..121: note = Antibody heavy Chain amino acid sequence
DVQLVESGGGLVQPGGSRKLSCVASGFSFSSFGMHWVRQAPEKGLEWVAYISSGSRIIYYEDKVKGRFTISRDNPKNTLFLQMTSLRSEDTAMYYCARSDRYDVYYSIDYWGQGTSVTVSS

SEQ ID NO: 236 - protein, 121 aa, synthetic construct
  REGION 1..121: note = Antibody heavy Chain amino acid sequence
DVQLVESGGGLVQPGGSRKLSCAASGFTFSTFGMHWIRQAPEKGLEWVAYISSDSRIIYYADTVRGRFSISRDNPKNTLFLQMTTLKSEDTAMYYCARSDRYDVYYSMNYWGQGTSVTVSS

SEQ ID NO: 237 - protein, 117 aa, synthetic construct
  REGION 1..117: note = Antibody heavy Chain amino acid sequence
EVQLQESGPELVKPGASVKMSCKASGYTFTNYVMHWVKQKPGQGLEWIGYINPDNDGANYNEKFKNKATLTSDKSSNTAYMELSSLTSEDSAVYYCARAARVWFPYWGQGTLVTVSA

SEQ ID NO: 238 - protein, 123 aa, synthetic construct
  REGION 1..123: note = Antibody heavy Chain amino acid sequence
EVQLQESGGGLVQPGGSLRLSCATSGFTFTDYYMSWVRQPPGKALEWLGFIRNKANGYTTEYSASVKGRFTISRDNSQSILYLQVNTLRAEDSATYYCARDPLYGRSYALDYWGQGTSVTVSS

SEQ ID NO: 239 - protein, 117 aa, synthetic construct
  REGION 1..117: note = Antibody heavy Chain amino acid sequence
EVQRQESGGGLVKPGGSLKLSCAASGFTFSSYAMSWVRQSPEKSLEWVAEISNGGSYTYYPDTVTGRFTISRDNAKNTLYLEMSSLRSEDTAMYYCARDRNGYFDYWGQGTSLTVSS

SEQ ID NO: 240 - protein, 121 aa, synthetic construct
  REGION 1..121: note = Antibody heavy Chain amino acid sequence
DVQLVESGGGLVRPGGSRRLSCVVSGFTFSSFGMHWVRQAPEKGLEWVAYISTGSSIIYYTDTVRGRFTISRDNPKNTLFLQMTSLRSEDTAIYYCARSDRYDVYYDMDYWGQGTSVTVSS

SEQ ID NO: 241 - protein, 121 aa, synthetic construct
  REGION 1..121: note = Antibody heavy Chain amino acid sequence
EFQLQQSGPELEKPGASVKISCRASGYSFTGYNMNWVKQNNGKSLEWIGNIDPYYGGTSYNQKFKGKATLTVDKSSSTAYMQLKSLTSEDSAVYYCARYYRYDVGGYFDVWGAGTTVTVSS

SEQ ID NO: 242 - protein, 117 aa, synthetic construct
  REGION 1..117: note = Antibody heavy Chain amino acid sequence
DVQLVESGGGLVQPGGSRKLSCAASGFTFSSFGMHWVRQAPEKGLEWVAYISSGNTTINYADTVKGRFTISRDNPKNTLFLQMTSLRSEDTAMYYCARYFFYAMDYWGQGTSVTVSS

SEQ ID NO: 243 - protein, 121 aa, synthetic construct
  REGION 1..121: note = Antibody heavy Chain amino acid sequence
DVQLVESGGGLVQPGGSRKLSCAASGFTFSTFGMHWVRQAPEKGLEWVAYISRGSIIIHYGDTVKGRFTISRDNPKNTLFLQMTSLRSEDTAMYYCVRSDRYDVYYDMDYWGQGTSVTVSS

SEQ ID NO: 244 - protein, 121 aa, synthetic construct
  REGION 1..121: note = Antibody heavy Chain amino acid sequence
DVQLVESGGGLVQPGGSRKLSCVVSGFTFSGFGMHWVRQAPEKGLEWVAYISTGSTIIYYTDTVRGRFTISRDNPKNTLFLQMTSLRSEDTAMYYCARSDRYDVYYDMDYWGQGTSVTVSS

SEQ ID NO: 245 - protein, 119 aa, synthetic construct
  REGION 1..119: note = Antibody heavy Chain amino acid sequence
EVKLEESGGGLVQPGGSMKISCTASGFTFTDAWMDWVRQSPEKGLEWVAEVRSKAKNYATYYPESVKGRFTISRDDSKSSVYLQMNNLRAEDTGVYYCTRSYVWYFDVWGAGTTVTVSS

SEQ ID NO: 246 - protein, 122 aa, synthetic construct
  REGION 1..122: note = Antibody heavy Chain amino acid sequence
QVQLQESGAELVRPGTSVKVSCKASGYAFTNYLIEWIKQRPGQGLEWIGVINPGSGGTNYNEKFKGKATLTADKSSSTAYMQLSSLTSDDSAVYFCARWDFDAGGLNLIAYWGQGTLVTVSA

SEQ ID NO: 247 - protein, 115 aa, synthetic construct
  REGION 1..115: note = Antibody heavy Chain amino acid sequence
QVQLQESGAEFVMPGAPVKLSCKASGYTFTNYWMNWVKQRPGRGLEWIGMIDPSDSETHYNQKFKDKATLTVDKSSSTAYIQLSSLTSEDSAVYYCASYDWFAYWGQGTLVTVSA

SEQ ID NO: 248 - protein, 300 aa, synthetic construct
  REGION 1..300: note = Antibody light Chain nucleic acid sequence
GATGTTGTTCTGACCCAAACTCCACTCTCTCTGCCTGTCAATATTGGAGATCAAGCCTCTATCTCTTGCAAGTCTACTAAGAGTCTTCTGAAAAGTGATGGATTCACTTATTTGGACTGGTTCCTGCAGAAGCCAGGCCAGTCTCCACAACTCCTAATATATTTGGTTTCTAATCGATTTTCTGGAATTCCAGACAGGTTCAGTGGCAGTGGGTCAGGAACAGATTTCACACTCAAGATCAGCAGAGTGGAGGCTGAGGATTTGGGAGTTTATTATTGCTTCCAGAGTATGGAAATAAAA

SEQ ID NO: 249 - protein, 321 aa, synthetic construct
  REGION 1..321: note = Antibody light Chain nucleic acid sequence
GATATCCAGATGACACAGTCTACATCCTCCCTGTCTGCCTCTCTGGGAGACAGAGTCACCATCAGTTGCAGTGCGAGTCAGGGCATTAGCAATTATTTAAACTGGTATCAACAGAAACCAGATGGAACTGTTAAACTCCTGATCTATTACTCATCAACTTTACACTCAGGAGTCCCATCAAGGTTCAGTGGCAGTGGGTCTGGGACAGATTATTCTCTCACCATCAACAACCTGGAACCTGAAGATATTGCCACTTACTATTGTCAACAGTATAGTAACCTTCCGTGGACGTTCGGTGGAGGCACCAAGCTGGAAATCAAA

SEQ ID NO: 250 - protein, 300 aa, synthetic construct; identical to SEQ ID NO: 248
  REGION 1..300: note = Antibody light Chain nucleic acid sequence

SEQ ID NO: 251 - protein, 318 aa, synthetic construct
  REGION 1..318: note = Antibody light Chain nucleic acid sequence
CAAATTGTTCTCACCCAGTCTCCAGCAATCATGTCTGCATCTCCAGGGGAGAAGGTCACCATAACCTGCAGTGCCAGTTCAAGTGTAAATTCCATGCACTGGTTCCAGCAGAAGTCAGGCACTTCTCCCAAACTCTGGATTTATAGCACATCCAACCTGGCTTCTGGAGTCCCTGCTCGCTTCAGTGGCAGTGGATCTGGGACCTCTTATTCTCTCACAATCAGCCGAATGGAGGCTGAAGATGCTGCCACTTATTACTGCCACCAAAGGAGTAGTTACCCACCCACGTTCGGTGCTGGGACCAAGCTGGAGCTGAAA

SEQ ID NO: 252 - protein, 300 aa, synthetic construct; identical to SEQ ID NO: 248
  REGION 1..300: note = Antibody light Chain nucleic acid sequence

SEQ ID NO: 253 - protein, 300 aa, synthetic construct; identical to SEQ ID NO: 248
  REGION 1..300: note = Antibody light Chain nucleic acid sequence

SEQ ID NO: 254 - protein, 318 aa, synthetic construct; identical to SEQ ID NO: 251
  REGION 1..318: note = Antibody light Chain nucleic acid sequence

SEQ ID NO: 255 - protein, 318 aa, synthetic construct
  REGION 1..318: note = Antibody light Chain nucleic acid sequence
CAAATTGTTCTCACCCAGTCTCCAGCAATCTTGTCTGCATCTCCAGGGGAGAAGGTCACCTTGACCTGCAGTGCCAGCTCAAGTGTTAATTACATGTACTGGTACCAGCAGAAGCCAGGATCCTCCCCCAGACTCCTGATTTATGACACCTCCGACCTGGCTTCTGGAGTCCCTATTCGCTTCAGTGGCAGTGGGTCTGGGACCTCTTACTCTCTCACAATCAGCCGAATGGAGGCTGAAGATGCTGCCACTTATTACTGCCAGCAGTGGAGTAGTTACCCACTCACGTTCGGTGCTGGGACCAAGCTGGACCTGAAA

SEQ ID NO: 256 - protein, 321 aa, synthetic construct
  REGION 1..321: note = Antibody light Chain nucleic acid sequence
GACATCAAGATGACCCAGTCTCCATCTTCCATGTATGCATCTCTAGGAGAGAGAGTCACTATCAGTTGCAAGGCGAGTCAGGACATTAATATGTACTTAAGCTGGTTCCAGCAGAAACCAGGGAAATCTCCTAAGACCCTGATCTATCGTGCAAACATTTTGGTAGATGGGGTCCCATCAAGGCTCAGTGGCAGTGGATCTGGGCAAGATTATTTTCTCACCATCAGCAGCCTGGAATATGAAGATTTGGGAATTTATTATTGTCTACAGTATGATGAATTTCCGCTCACGGTCGGTGCTGGGACCAAAGCTGGAGCTGAA

SEQ ID NO: 257 - protein, 318 aa, synthetic construct
  REGION 1..318: note = Antibody light Chain nucleic acid sequence
CAAATTGTTCTCACCCAGTCTCCAGCAATCATGTCTACATCTCCAGGGGAGAAGGTCACCATATCCTGCAGTGCCAGCTCAAGTGTAAGTTACATGAACTGGTACCAACAGAAACCAGGATCCTCCCCCAAACCCTGGATTCATCACACATCCAATTTGGCTTCTGGAGTCCCTGCTCGCTTCAGTGGCAGTGGGTCTGGGACCTCTTACTCTCTCACAATCAGCAGCATGGAGGCTGAAGATGCTGCCACCTATTACTGCCAACAGTATCATAGTTACCCACTCACGTTCGGTGCTGGGACCAAGCTGGAGCTGAAA

SEQ ID NO: 258 - protein, 300 aa, synthetic construct; identical to SEQ ID NO: 248
  REGION 1..300: note = Antibody light Chain nucleic acid sequence

SEQ ID NO: 259 - protein, 339 aa, synthetic construct
  REGION 1..339: note = Antibody light Chain nucleic acid sequence
GATGTTGTGATGACCCAAACTCCACTCTCCCTGCCTGTCAGTCTTGGAGATCAAGCCTCCATCTCTTGCAGATCTAGTCAGAGCCTTGTACACAGTAATGGAAACACCTATTTACATTGGTACCTGCAGAAGCCAGGCCAGTCTCCAAAGCTCCTGATCTTCAAAGTTTCCAACCGATTTTCTGGGGTCCCAGACAGGTTCAGTGGCAGTGGATCAGGGACAGATTTCACACTCAAGATCAGCAGAGTGGAGGCTGAGGATCTGGGAGTTTATTTCTGCTCTCAAAGTACACATGTTCCTCCGCTCACGTTCGGTGCTGGGACCAAGCTGGAGCTGAAA

SEQ ID NO: 260 - protein, 321 aa, synthetic construct
  REGION 1..321: note = Antibody light Chain nucleic acid sequence
GACATCAAGATGACCCAGTCTCCATCTTCCATGTACGCATCTCTAGGAGAGAGAGTCACTATCACTTGCAAGGCGAGTCAGGACATTAATATCTATTTAAGCTGGTTGCAGCAGAAACCAGGGAAATCTCCTAAGACCCTGATCTATCGAACAAACAGATTGTTAGATGGGGTCCCATCAAGGTTCAGTGGCGGTGGATCTGGCCAAGATTATTCTCTCACCATCAGCAGCCTGGAATATGAAGATATGGGAATTTATTATTGTCTACAGTATGATGAGTTTCCGCTCACGTTCGGTGCTGGGACCAAGCTGGAGCTGAAA

SEQ ID NO: 261 - protein, 321 aa, synthetic construct; identical to SEQ ID NO: 260
  REGION 1..321: note = Antibody light Chain nucleic acid sequence

SEQ ID NO: 262 - protein, 318 aa, synthetic construct
  REGION 1..318: note = Antibody light Chain nucleic acid sequence
CAAATTGTTCTCACCCAGTCTCCAACAATCATGTCTGCATCTCCAGGGGAGAAGGTCACCATATCCTGCAGTGCCAGCTCAAGTGTAAGTTACATGTACTGGTACCAGCAGAAGCCAGGATCCTCCCCCAAACCCTGGATTTATCGCACATCCAACCTGGCTTCTGGAGTCCCTCCTCGCTTCAGTGGCAGTGGGTCTGGGACCTCTTACTCTCTCACAATCAGCAGCATGGAGGCTGAAGATGCTGCCACTTATTACTGCCAGCAGTATCATAGTTACCCGTACACGTTCGGAGGGGGGACCAAGCTGGAAATAAAA

SEQ ID NO: 263 - protein, 321 aa, synthetic construct
  REGION 1..321: note = Antibody light Chain nucleic acid sequence
CAAATTGTTCTCACCCAGTCTCCAGCAATCATGTCTGCATCTCCAGGGGAGAAGGTCACCATGACCTGTAGTGCCAGCTCAAGTGTAAATTACATGCACTGGTACCAGCAGAAGTCAGGCACCTCCCCCAAAAGATGGATTTATGACACATCCAAACTGGCTTCTGGAGTCCCTGCTCGCTTCAGTGGCAGTGGGTCTGGGACCTCTTACTCTCTCACAATCAGCAGCATGGAGGCTGAAGATGCTGCCACTTATTACTGCCAGCAGTGGAGTAGTAACCCACCGCTCACGTTCGGTGCTGGGACCAAGCTGGAGCTGAAA

SEQ ID NO: 264 - protein, 318 aa, synthetic construct; identical to SEQ ID NO: 255
  REGION 1..318: note = Antibody light Chain nucleic acid sequence

SEQ ID NO: 265 - protein, 318 aa, synthetic construct
  REGION 1..318: note = Antibody light Chain nucleic acid sequence
CAAATTGTTCTCACCCAGTCTCCAGCAATCATGTCTGCATCTCCAGGGGAGAAGGTCAGCATATCCTGCAGTGCCAGCTCAAGTGTCAGTTACATGTACTGGTACCAGCAGAAGCCAGGATCCTCCCCCAAACCCTGGATTCATCGCACATCCACCTTGGCTTCTGGAGTCCCTGCTCGCTTCAGTGGCAGTGGGTCTGGGACTTCTTACTCTCTCACAATCAGCAGCATGGAGGCTGAAGATGCTGCCACTTATTACTGCCAGCAGTATCATAGTGACCCGCTCACGTTCGGTGCTGGGACCAAGCTGGAGCTGAAA

SEQ ID NO: 266 - protein, 339 aa, synthetic construct; identical to SEQ ID NO: 259
  REGION 1..339: note = Antibody light Chain nucleic acid sequence

SEQ ID NO: 267 - protein, 336 aa, synthetic construct
  REGION 1..336: note = Antibody light Chain nucleic acid sequence
GATGTTTTGATGACCCAAACTCCACTCTCCCTGCCTGTCAGTCTTGGAGATCAAGCCTCCATCTCTTGCAGATCTAGTCAGAGCATTGTACATAGTAATGGAAACACCTATTTAGAATGGTACCTGCAGAAACCAGGCCAGTCTCCAAAGCTCCTGATCTACAAAGTTTCCAACCGATTTTCTGGGGTCCCAGACAGGTTCAGTGGCAGTGGATCAGGGACAGATTTCACACTCAAGATCAGAAGAGTGGAGGCTGAGGATCTGGGAGTTTATTACTGCTTTCAAGGTTCACATGTTCCGTACACGTTCGGAGGGGGGACCAAGCTGGAAATAAAA

SEQ ID NO: 268 - protein, 318 aa, synthetic construct
  REGION 1..318: note = Antibody light Chain nucleic acid sequence
CAAATTGTTCTCACCCAGTCTCCAGCAATCATGTCTGCATCTCCAGGGGAGAAGGTCACCATGACCTGCAGTGCCAGCTCAAGAGTGAATTACATGCACTGGTACCAGCAGAAGTCAGGCACTTCCCCCAAAAGATGGATTTATGACACATCCAAACTGGCTTCTGGAGTCCCTGCTCGCTTCAGTGGCAGTGGGTCTGGGACCTCTTTCTCTCTCACAATCAGCAGCATGGAGGCTGAAGATGCTGCCACTTATTACTGCCAGCAGTGGAGTACTAACCCACCCACGTTCGGAGGGGGGACCAAGCTGGAAATAAAA

SEQ ID NO: 269 - protein, 318 aa, synthetic construct; identical to SEQ ID NO: 268
  REGION 1..318: note = Antibody light Chain nucleic acid sequence

SEQ ID NO: 270 - protein, 330 aa, synthetic construct
  REGION 1..330: note = Antibody light Chain nucleic acid sequence
CAAATTGTTCTCACCCAGTCTCCAGCAATCATGTCTGCATCTCTAGGGGAACGGGTCACCATGACCTGCACTGCCAGCTCAAGTGTTCGTTCCAGTTACTTGCACTGGTTCCAGCGGAAACCAGGATCCTCCCCCAAACTCTGGATTTATAGCACATCCGACCTGGCTTCTGGAGTCCCAGCTCGCTTCAGTGGCAGTGGGTCTGGGACCTCTTTCTCTCTCACAATCAGCAGCATGGAGGCTGAAGATGCTGCCACTTATTACTGCCACCAGTATCATCGTTCCCCACCCACGTGGACGTTCGGTGGAGGCACCAAGCTGGAGATCAAA

SEQ ID NO: 271 - protein, 339 aa, synthetic construct
  REGION 1..339: note = Antibody light Chain nucleic acid sequence
GACATTGTGATGACACAGTCTCCATCCTCCCTGACTGTGACAGCAGGAGAGAAGGTCACTATGACCTGCAAGTCCAGTCAGAGTCTGTTCAACAGTGGAAATCAAAAGAACTACTTGACCTGGTACCAGCAGAAACCAGGGCAGCCTCCTAAACTGTTGATCTACTGGGCATCCACTAGGGAATCTGGGGTCCCTGATCGCTTCACAGGCAGTGGATCTGGAACAGATTTCACTCTCACCATCAGCAGTGTGCAGGCTGAAGACCTGGCAGTTTATTACTGTCAGAATGATTATAGTTATCCGTGGACGTTCGGTGGAGGCACCAAGCTGGAAATCAAA

SEQ ID NO: 272 - protein, 318 aa, synthetic construct
  REGION 1..318: note = Antibody light Chain nucleic acid sequence
CAAATTGTTCTCACCCAGTCTCCAGCACTCATGTCTGCATCTCCAGGGGAGAAGGTCACCATGACCTGCAGTGCCAGCTCAAGTGTAAGTTACATGTACTGGTACCAGCAGAAGCCAAGATCCTCCCCCAAACCCTGGATTTATCTCACATCCAACCTGGCTTCTGGAGTCCCTGCTCGCTTCAGTGGCAGTGGGTCTGGGACCTCTTACTCTCTCACAATCAGCAGCATGGAGGCTGAAGATGCTGCCACTTATTACTGCCAGCAGTGGAGTAGTAACCCACTCACGTTCGGTGCTGGGACCAAGCTGGAGCTGAAA

SEQ ID NO: 273 - protein, 318 aa, synthetic construct
  REGION 1..318: note = Antibody light Chain nucleic acid sequence
GAAATTTTGCTCACCCAGTCTCCAGCAATCATAGCTGCATCTCCTGGGGAGAAGGTCACCATCACCTGCAGTGGCAGTTCAACTGTAAGTTACATGAATTGGTACCAGCAGAAACCAGGATCCTCCCCCAAAATATGGATTTATTATATATCCAAGTTGGCTTCTGGAGTTCCTCCTCGCTTCAGTGGCAGTGGGTCTGGGACATCTTTCTCTTTCACAATCAACAGCATGGAGGCTGAAGATGTTGCCACTTATTACTGTCAGCAAAGGAGTAGTTTCCCATTCACGTTCGGCTCGGGGACAAAGTTGGAAATAAAA

SEQ ID NO: 274 - protein, 318 aa, synthetic construct; identical to SEQ ID NO: 251
  REGION 1..318: note = Antibody light Chain nucleic acid sequence

SEQ ID NO: 275 - protein, 321 aa, synthetic construct
  REGION 1..321: note = Antibody light Chain nucleic acid sequence
GATATCCAGATGACACAGACTACATCCTCCCTGTCTGCCTCTCTGGGAGACAGAGTCACCATCAGTTGCAGGGCAACTCAGGACATTACCAATTTTTTAAACTGGTATCAGCAGAAACCAGATGGAACTGTTAAACTCCTGATTTCCTACACATCAACATTACATTCAGGAGTCCCATCAAGGTTCAGTGGCAGTGGGTCTGGAACAGATTATTCTCTCACCATTAGTAACCTGGAACAAGAAGATGTTGCCACTTACTTTTGCCAACAGGTTAATACGCTTCCTCCGACGTTCGGTGGAGGCACCAAGCTGGAAATCAAA

SEQ ID NO: 276 - protein, 318 aa, synthetic construct
  REGION 1..318: note = Antibody light Chain nucleic acid sequence
CAAATTGTTCTCACCCAGTCTCCAGCAATCATGTCTGCATCTCCAGGGGAGAAGGTCACCATAACCTGCAGTGCCAGCTCAAGTGTAAGTTACATGCACTGGTTCAAGCAGAAGCCAGGCACTTCTCCCAAAGTCTGGATTTATAGCACATCCAACCTGGCTTCTGGAGTCCCTACTCGCTTCAGTGGCAGTGGATCTGGGACCTCTTACTCTCTCACAATCAGCCGAATGGAGGCTGAAGATGCTGCCACTTATTACTGCCAGCAAAGGAGTAGTTACCCAGTGACGTTCGGTGGAGGCACCAAGCTGGAAATCAAA

SEQ ID NO: 277 - protein, 321 aa, synthetic construct
  REGION 1..321: note = Antibody light Chain nucleic acid sequence
GACATCAAGATGACCCAGTCTCCATCTTCCATGTATACATCTCTAGGAGAGAGAGTCACTATCACTTGCAAGGCGAGTCAGGACATTAATAACTATTTAAACTGGTTCCAGCAGAAACCAGGGAAATCTCCTAAGACCCTGATCTATCGTGCAAACAGATTGGTAGATGGGGTCCCATCAAGGTTCAGTGGCAGTGGATCTGGGCAAGATTATTCTCTCACCATCAGCAGCCTGGAGTTTGAAGATGTGGGAATTTATTATTGTCTACAGTATGAAGAGTTTCCGCTCACGTTCGGTGCTGGGACCAAGCTGGAGCTGAAA

SEQ ID NO: 278 - protein, 330 aa, synthetic construct; identical to SEQ ID NO: 270
  REGION 1..330: note = Antibody light Chain nucleic acid sequence

SEQ ID NO: 279 - protein, 112 aa, synthetic construct
  REGION 1..112: note = Antibody light Chain amino acid sequence
DVVLTQTPLSLPVNIGDQASISCKSTKSLLKSDGFTYLDWFLQKPGQSPQLLIYLVSNRFSGIPDRFSGSGSGTDFTLKISRVEAEDLGVYYCFQSNYLPYTFGGGTKLEIK

SEQ ID NO: 280 - protein, 107 aa, synthetic construct
  REGION 1..107: note = Antibody light Chain amino acid sequence
DIQMTQSTSSLSASLGDRVTISCSASQGISNYLNWYQQKPDGTVKLLIYYSSTLHSGVPSRFSGSGSGTDYSLTINNLEPEDIATYYCQQYSNLPWTFGGGTKLEIK

SEQ ID NO: 281 - protein, 112 aa, synthetic construct; identical to SEQ ID NO: 279
  REGION 1..112: note = Antibody light Chain amino acid sequence

SEQ ID NO: 282 - protein, 106 aa, synthetic construct
  REGION 1..106: note = Antibody light Chain amino acid sequence
QIVLTQSPAIMSASPGEKVTITCSASSSVNSMHWFQQKSGTSPKLWIYSTSNLASGVPARFSGSGSGTSYSLTISRMEAEDAATYYCHQRSSYPPTFGAGTKLELK

SEQ ID NO: 283 - protein, 112 aa, synthetic construct; identical to SEQ ID NO: 279
  REGION 1..112: note = Antibody light Chain amino acid sequence

SEQ ID NO: 284 - protein, 112 aa, synthetic construct; identical to SEQ ID NO: 279
  REGION 1..112: note = Antibody light Chain amino acid sequence

SEQ ID NO: 285 - protein, 106 aa, synthetic construct; identical to SEQ ID NO: 282
  REGION 1..106: note = Antibody light Chain amino acid sequence

SEQ ID NO: 286 - protein, 106 aa, synthetic construct
  REGION 1..106: note = Antibody light Chain amino acid sequence
QIVLTQSPAILSASPGEKVTLTCSASSSVNYMYWYQQKPGSSPRLLIYDTSDLASGVPIRFSGSGSGTSYSLTISRMEAEDAATYYCQQWSSYPLTFGAGTKLDLK

SEQ ID NO: 287 - protein, 107 aa, synthetic construct
  REGION 1..107: note = Antibody light Chain amino acid sequence
DIKMTQSPSSMYASLGERVTISCKASQDINMYLSWFQQKPGKSPKTLIYRANILVDGVPSRLSGSGSGQDYFLTISSLEYEDLGIYYCLQYDEFPLTVGAGTKAGAE

SEQ ID NO: 288 - protein, 106 aa, synthetic construct
  REGION 1..106: note = Antibody light Chain amino acid sequence
QIVLTQSPAIMSTSPGEKVTISCSASSSVSYMNWYQQKPGSSPKPWIHHTSNLASGVPARFSGSGSGTSYSLTISSMEAEDAATYYCQQYHSYPLTFGAGTKLELK

SEQ ID NO: 289 - protein, 112 aa, synthetic construct; identical to SEQ ID NO: 279
  REGION 1..112: note = Antibody light Chain amino acid sequence

SEQ ID NO: 290 - protein, 113 aa, synthetic construct
  REGION 1..113: note = Antibody light Chain amino acid sequence
DVVMTQTPLSLPVSLGDQASISCRSSQSLVHSNGNTYLHWYLQKPGQSPKLLIFKVSNRFSGVPDRFSGSGSGTDFTLKISRVEAEDLGVYFCSQSTHVPPLTFGAGTKLELK

SEQ ID NO: 291 - protein, 107 aa, synthetic construct
  REGION 1..107: note = Antibody light Chain amino acid sequence
DIKMTQSPSSMYASLGERVTITCKASQDINIYLSWLQQKPGKSPKTLIYRTNRLLDGVPSRFSGGGSGQDYSLTISSLEYEDMGIYYCLQYDEFPLTFGAGTKLELK

SEQ ID NO: 292 - protein, 107 aa, synthetic construct; identical to SEQ ID NO: 291
  REGION 1..107: note = Antibody light Chain amino acid sequence

SEQ ID NO: 293 - protein, 106 aa, synthetic construct
  REGION 1..106: note = Antibody light Chain amino acid sequence
QIVLTQSPTIMSASPGEKVTISCSASSSVSYMYWYQQKPGSSPKPWIYRTSNLASGVPPRFSGSGSGTSYSLTISSMEAEDAATYYCQQYHSYPYTFGGGTKLEIK

SEQ ID NO: 294 - protein, 107 aa, synthetic construct
  REGION 1..107: note = Antibody light Chain amino acid sequence
QIVLTQSPAIMSASPGEKVTMTCSASSSVNYMHWYQQKSGTSPKRWIYDTSKLASGVPARFSGSGSGTSYSLTISSMEAEDAATYYCQQWSSNPPLTFGAGTKLELK

SEQ ID NO: 295 - protein, 106 aa, synthetic construct; identical to SEQ ID NO: 286
  REGION 1..106: note = Antibody light Chain amino acid sequence

SEQ ID NO: 296 - protein, 106 aa, synthetic construct
  REGION 1..106: note = Antibody light Chain amino acid sequence
QIVLTQSPAIMSASPGEKVSISCSASSSVSYMYWYQQKPGSSPKPWIHRTSTLASGVPARFSGSGSGTSYSLTISSMEAEDAATYYCQQYHSDPLTFGAGTKLELK

SEQ ID NO: 297 - protein, 113 aa, synthetic construct; identical to SEQ ID NO: 290
  REGION 1..113: note = Antibody light Chain amino acid sequence

SEQ ID NO: 298 - protein, 112 aa, synthetic construct
  REGION 1..112: note = Antibody light Chain amino acid sequence
DVLMTQTPLSLPVSLGDQASISCRSSQSIVHSNGNTYLEWYLQKPGQSPKLLIYKVSNRFSGVPDRFSGSGSGTDFTLKIRRVEAEDLGVYYCFQGSHVPYTFGGGTKLEIK

SEQ ID NO: 299 - protein, 106 aa, synthetic construct
  REGION 1..106: note = Antibody light Chain amino acid sequence
QIVLTQSPAIMSASPGEKVTMTCSASSRVNYMHWYQQKSGTSPKRWIYDTSKLASGVPARFSGSGSGTSFSLTISSMEAEDAATYYCQQWSTNPPTFGGGTKLEIK

SEQ ID NO: 300 - protein, 106 aa, synthetic construct; identical to SEQ ID NO: 299
  REGION 1..106: note = Antibody light Chain amino acid sequence

SEQ ID NO: 301 - protein, 110 aa, synthetic construct
  REGION 1..110: note = Antibody light Chain amino acid sequence
QIVLTQSPAIMSASLGERVTMTCTASSSVRSSYLHWFQRKPGSSPKLWIYSTSDLASGVPARFSGSGSGTSFSLTISSMEAEDAATYYCHQYHRSPPTWTFGGGTKLEIK

SEQ ID NO: 302 - protein, 113 aa, synthetic construct
  REGION 1..113: note = Antibody light Chain amino acid sequence
DIVMTQSPSSLTVTAGEKVTMTCKSSQSLFNSGNQKNYLTWYQQKPGQPPKLLIYWASTRESGVPDRFTGSGSGTDFTLTISSVQAEDLAVYYCQNDYSYPWTFGGGTKLEIK

SEQ ID NO: 303 - protein, 106 aa, synthetic construct
  REGION 1..106: note = Antibody light Chain amino acid sequence
QIVLTQSPALMSASPGEKVTMTCSASSSVSYMYWYQQKPRSSPKPWIYLTSNLASGVPARFSGSGSGTSYSLTISSMEAEDAATYYCQQWSSNPLTFGAGTKLELK

SEQ ID NO: 304 - protein, 106 aa, synthetic construct
  REGION 1..106: note = Antibody light Chain amino acid sequence
EILLTQSPAIIAASPGEKVTITCSGSSTVSYMNWYQQKPGSSPKIWIYYISKLASGVPPRFSGSGSGTSFSFTINSMEAEDVATYYCQQRSSFPFTFGSGTKLEIK

SEQ ID NO: 305 - protein, 106 aa, synthetic construct; identical to SEQ ID NO: 282
  REGION 1..106: note = Antibody light Chain amino acid sequence

SEQ ID NO: 306 - protein, 107 aa, synthetic construct
  REGION 1..107: note = Antibody light Chain amino acid sequence
DIQMTQTTSSLSASLGDRVTISCRATQDITNFLNWYQQKPDGTVKLLISYTSTLHSGVPSRFSGSGSGTDYSLTISNLEQEDVATYFCQQVNTLPPTFGGGTKLEIK

SEQ ID NO: 307 - protein, 106 aa, synthetic construct
  REGION 1..106: note = Antibody light Chain amino acid sequence
QIVLTQSPAIMSASPGEKVTITCSASSSVSYMHWFKQKPGTSPKVWIYSTSNLASGVPTRFSGSGSGTSYSLTISRMEAEDAATYYCQQRSSYPVTFGGGTKLEIK

SEQ ID NO: 308 - protein, 107 aa, synthetic construct
  REGION 1..107: note = Antibody light Chain amino acid sequence
DIKMTQSPSSMYTSLGERVTITCKASQDINNYLNWFQQKPGKSPKTLIYRANRLVDGVPSRFSGSGSGQDYSLTISSLEFEDVGIYYCLQYEEFPLTFGAGTKLELK

SEQ ID NO: 309 - protein, 110 aa, synthetic construct; identical to SEQ ID NO: 301
  REGION 1..110: note = Antibody light Chain amino acid sequence

SEQ ID NO: 310 - protein, 8 aa, synthetic construct
  REGION 1..8: note = Antibody Complementarity-determining regions (CDRs)
GYTFSSYW

SEQ ID NO: 311 - protein, 8 aa, synthetic construct
  REGION 1..8: note = Antibody Complementarity-determining regions (CDRs)
ILPGSGST

SEQ ID NO: 312 - protein, 12 aa, synthetic construct
  REGION 1..12: note = Antibody Complementarity-determining regions (CDRs)
TSYFGSSSSFAY

SEQ ID NO: 313 - protein, 11 aa, synthetic construct
  REGION 1..11: note = Antibody Complementarity-determining regions (CDRs)
KSLLKSDGFTY

SEQ ID NO: 314 - skipped

SEQ ID NO: 315 - protein, 9 aa, synthetic construct
  REGION 1..9: note = Antibody Complementarity-determining regions (CDRs)
FQSNYLPYT

SEQ ID NO: 316 - protein, 8 aa, synthetic construct
  REGION 1..8: note = Antibody Complementarity-determining regions (CDRs)
GYTFIDHG

SEQ ID NO: 317 - protein, 8 aa, synthetic construct
  REGION 1..8: note = Antibody Complementarity-determining regions (CDRs)
ISPGNGDI

SEQ ID NO: 318 - protein, 13 aa, synthetic construct
  REGION 1..13: note = Antibody Complementarity-determining regions (CDRs)
KRGVRRGGYAMDY

SEQ ID NO: 319 - protein, 6 aa, synthetic construct
  REGION 1..6: note = Antibody Complementarity-determining regions (CDRs)
QGISNY

SEQ ID NO: 320 - skipped

SEQ ID NO: 321 - protein, 9 aa, synthetic construct
  REGION 1..9: note = Antibody Complementarity-determining regions (CDRs)
QQYSNLPWT

SEQ ID NO: 322 - protein, 8 aa, synthetic construct
  REGION 1..8: note = Antibody Complementarity-determining regions (CDRs)
GYTFTSYW

SEQ ID NO: 323 - protein, 8 aa, synthetic construct
  REGION 1..8: note = Antibody Complementarity-determining regions (CDRs)
INPSNGAT

SEQ ID NO: 324 - protein, 6 aa, synthetic construct
  REGION 1..6: note = Antibody Complementarity-determining regions (CDRs)
TRGSPY

SEQ ID NO: 325 - protein, 11 aa, synthetic construct
  REGION 1..11: note = Antibody Complementarity-determining regions (CDRs)
KSLLKSDGFTY

SEQ ID NO: 326 - skipped

SEQ ID NO: 327 - protein, 9 aa, synthetic construct
  REGION 1..9: note = Antibody Complementarity-determining regions (CDRs)
FQSNYLPYT

SEQ ID NO: 328 - protein, 8 aa, synthetic construct
  REGION 1..8: note = Antibody Complementarity-determining regions (CDRs)
GYTFTTYW

SEQ ID NO: 329 - protein, 8 aa, synthetic construct
  REGION 1..8: note = Antibody Complementarity-determining regions (CDRs)
IDPLDSYA

SEQ ID NO: 330 - protein, 14 aa, synthetic construct
  REGION 1..14: note = Antibody Complementarity-determining regions (CDRs)
TRGGDGDYYFSLDY

SEQ ID NO: 331 - protein, 4 aa, synthetic construct
  REGION 1..4: note = Antibody Complementarity-determining regions (CDRs)
SSVN

SEQ ID NO: 332 - skipped

SEQ ID NO: 333 - protein, 9 aa, synthetic construct
  REGION 1..9: note = Antibody Complementarity-determining regions (CDRs)
HQRSSYPPT

SEQ ID NO: 334 - protein, 8 aa, synthetic construct
  REGION 1..8: note = Antibody Complementarity-determining regions (CDRs)
GYSFTGYN

SEQ ID NO: 335 - protein, 8 aa, synthetic construct
  REGION 1..8: note = Antibody Complementarity-determining regions (CDRs)
IDPYYGGA

SEQ ID NO: 336 - protein, 11 aa, synthetic construct
  REGION 1..11: note = Antibody Complementarity-determining regions (CDRs)
ARGDDYVDFAY

SEQ ID NO: 337 - protein, 11 aa, synthetic construct
  REGION 1..11: note = Antibody Complementarity-determining regions (CDRs)
KSLLKSDGFTY

SEQ ID NO: 338 - skipped

SEQ ID NO: 339 - protein, 9 aa, synthetic construct
  REGION 1..9: note = Antibody Complementarity-determining regions (CDRs)
FQSNYLPYT

SEQ ID NO: 340 - protein, 8 aa, synthetic construct
  REGION 1..8: note = Antibody Complementarity-determining regions (CDRs)
GFSLTDYG

SEQ ID NO: 341 - protein, 7 aa, synthetic construct
  REGION 1..7: note = Antibody Complementarity-determining regions (CDRs)
IWGGGST

SEQ ID NO: 342 - protein, 13 aa, synthetic construct
  REGION 1..13: note = Antibody Complementarity-determining regions (CDRs)
AKHYPGNYSAMDY

SEQ ID NO: 343 - protein, 11 aa, synthetic construct
  REGION 1..11: note = Antibody Complementarity-determining regions (CDRs)
KSLLKSDGFTY

SEQ ID NO: 344 - skipped

SEQ ID NO: 345 - protein, 9 aa, synthetic construct
  REGION 1..9: note = Antibody Complementarity-determining regions (CDRs)
FQSNYLPYT

SEQ ID NO: 346 - protein, 8 aa, synthetic construct
  REGION 1..8: note = Antibody Complementarity-determining regions (CDRs)
GYTFSLYI

SEQ ID NO: 347 - protein, 8 aa, synthetic construct
  REGION 1..8: note = Antibody Complementarity-determining regions (CDRs)
FNPDNDDT

SEQ ID NO: 348 - protein, 13 aa, synthetic construct
  REGION 1..13: note = Antibody Complementarity-determining regions (CDRs)
TREDYYGFWYFDV

SEQ ID NO: 349 - protein, 4 aa, synthetic construct
  REGION 1..4: note = Antibody Complementarity-determining regions (CDRs)
SSVN

SEQ ID NO: 350 - skipped

SEQ ID NO: 351 - protein, 9 aa, synthetic construct
  REGION 1..9: note = Antibody Complementarity-determining regions (CDRs)
HQRSSYPPT

SEQ ID NO: 352 - protein, 8 aa, synthetic construct
  REGION 1..8: note = Antibody Complementarity-determining regions (CDRs)
GDSITSGC

SEQ ID NO: 353 - protein, 7 aa, synthetic construct
  REGION 1..7: note = Antibody Complementarity-determining regions (CDRs)
ISYSGIT

SEQ ID NO: 354 - protein, 13 aa, synthetic construct
  REGION 1..13: note = Antibody Complementarity-determining regions (CDRs)
SRSSTMITTGFAY

SEQ ID NO: 355 - protein, 5 aa, synthetic construct
  REGION 1..5: note = Antibody Complementarity-determining regions (CDRs)
SSVNY

SEQ ID NO: 356 - skipped

SEQ ID NO: 357 - protein, 9 aa, synthetic construct
  REGION 1..9: note = Antibody Complementarity-determining regions (CDRs)
QQWSSYPLT

SEQ ID NO: 358 - protein, 8 aa, synthetic construct
  REGION 1..8: note = Antibody Complementarity-determining regions (CDRs)
GYTFTTYG

SEQ ID NO: 359 - protein, 8 aa, synthetic construct
  REGION 1..8: note = Antibody Complementarity-determining regions (CDRs)
INTYSGVP

SEQ ID NO: 360 - protein, 10 aa, synthetic construct
  REGION 1..10: note = Antibody Complementarity-determining regions (CDRs)
TRSYSYYFDI

SEQ ID NO: 361 - protein, 6 aa, synthetic construct
  REGION 1..6: note = Antibody Complementarity-determining regions (CDRs)
QDINMY

SEQ ID NO: 362 - skipped

SEQ ID NO: 363 - protein, 9 aa, synthetic construct
  REGION 1..9: note = Antibody Complementarity-determining regions (CDRs)
LQYDEFPLT

SEQ ID NO: 364 - protein, 8 aa, synthetic construct
  REGION 1..8: note = Antibody Complementarity-determining regions (CDRs)
GFSLTGYG

SEQ ID NO: 365 - protein, 7 aa, synthetic construct
  REGION 1..7: note = Antibody Complementarity-determining regions (CDRs)
IWGDGST

SEQ ID NO: 366 - protein, 15 aa, synthetic construct
  REGION 1..15: note = Antibody Complementarity-determining regions (CDRs)
ARGWESLSTMSWFAY

SEQ ID NO: 367 - protein, 5 aa, synthetic construct
  REGION 1..5: note = Antibody Complementarity-determining regions (CDRs)
SSVSY

SEQ ID NO: 368 - skipped

SEQ ID NO: 369 - protein, 9 aa, synthetic construct
  REGION 1..9: note = Antibody Complementarity-determining regions (CDRs)
QQYHSYPLT

SEQ ID NO: 370 - protein, 8 aa, synthetic construct
  REGION 1..8: note = Antibody Complementarity-determining regions (CDRs)
GYTFTLYI

SEQ ID NO: 371 - protein, 8 aa, synthetic construct
  REGION 1..8: note = Antibody Complementarity-determining regions (CDRs)
FNPDNDDT

SEQ ID NO: 372 - protein, 12 aa, synthetic construct
  REGION 1..12: note = Antibody Complementarity-determining regions (CDRs)
AREDYYGFWYFD

SEQ ID NO: 373 - protein, 11 aa, synthetic construct
  REGION 1..11: note = Antibody Complementarity-determining regions (CDRs)
KSLLKSDGFTY

SEQ ID NO: 374 - skipped

SEQ ID NO: 375 - protein, 9 aa, synthetic construct
  REGION 1..9: note = Antibody Complementarity-determining regions (CDRs)
FQSNYLPYT

SEQ ID NO: 376 - protein, 8 aa, synthetic construct
  REGION 1..8: note = Antibody Complementarity-determining regions (CDRs)
GFTFSGFG

SEQ ID NO: 377 - protein, 8 aa, synthetic construct
  REGION 1..8: note = Antibody Complementarity-determining regions (CDRs)
ISSGSNII

SEQ ID NO: 378 - protein, 14 aa, synthetic construct
  REGION 1..14: note = Antibody Complementarity-determining regions (CDRs)
ARSDRYDVYYDMDY

SEQ ID NO: 379 - protein, 11 aa, synthetic construct
  REGION 1..11: note = Antibody Complementarity-determining regions (CDRs)
QSLVHSNGNTY

SEQ ID NO: 380 - skipped

SEQ ID NO: 381 - protein, 10 aa, synthetic construct
  REGION 1..10: note = Antibody Complementarity-determining regions (CDRs)
SQSTHVPPLT

SEQ ID NO: 382 - protein, 8 aa, synthetic construct
  REGION 1..8: note = Antibody Complementarity-determining regions (CDRs)
GYAFSTSW

SEQ ID NO: 383 - protein, 8 aa, synthetic construct
  REGION 1..8: note = Antibody Complementarity-determining regions (CDRs)
IYPGDGDD

SEQ ID NO: 384 - protein, 10 aa, synthetic construct
  REGION 1..10: note = Antibody Complementarity-determining regions (CDRs)
ASYDGYSFAY

SEQ ID NO: 385 - protein, 6 aa, synthetic construct
  REGION 1..6: note = Antibody Complementarity-determining regions (CDRs)
QDINIY

SEQ ID NO: 386 - skipped

SEQ ID NO: 387 - protein, 9 aa, synthetic construct
  REGION 1..9: note = Antibody Complementarity-determining regions (CDRs)
LQYDEFPLT

SEQ ID NO: 388 - protein, 8 aa, synthetic construct
  REGION 1..8: note = Antibody Complementarity-determining regions (CDRs)
GFTFSTFG

SEQ ID NO: 389 - protein, 8 aa, synthetic construct
  REGION 1..8: note = Antibody Complementarity-determining regions (CDRs)
ISSGSSII

SEQ ID NO: 390 - protein, 9 aa, synthetic construct
  REGION 1..9: note = Antibody Complementarity-determining regions (CDRs)
ARSDRYDVY

SEQ ID NO: 391 - protein, 6 aa, synthetic construct
  REGION 1..6: note = Antibody Complementarity-determining regions (CDRs)
QDINIY

SEQ ID NO: 392 - skipped

SEQ ID NO: 393 - protein, 9 aa, synthetic construct
  REGION 1..9: note = Antibody Complementarity-determining regions (CDRs)
LQYDEFPLT

SEQ ID NO: 394 - protein, 8 aa, synthetic construct
  REGION 1..8: note = Antibody Complementarity-determining regions (CDRs)
GDSITSGY

SEQ ID NO: 395 - protein, 7 aa, synthetic construct
  REGION 1..7: note = Antibody Complementarity-determining regions (CDRs)
IRYSGST

SEQ ID NO: 396 - protein, 15 aa, synthetic construct
  REGION 1..15: note = Antibody Complementarity-determining regions (CDRs)
ARYVVRLPHYYAMDY

SEQ ID NO: 397 - protein, 5 aa, synthetic construct
  REGION 1..5: note = Antibody Complementarity-determining regions (CDRs)
SSVSY

SEQ ID NO: 398 - skipped

SEQ ID NO: 399 - protein, 9 aa, synthetic construct
  REGION 1..9: note = Antibody Complementarity-determining regions (CDRs)
QQYHSYPYT

SEQ ID NO: 400 - protein, 8 aa, synthetic construct
  REGION 1..8: note = Antibody Complementarity-determining regions (CDRs)
GYAFTNYL

SEQ ID NO: 401 - protein, 8 aa, synthetic construct
  REGION 1..8: note = Antibody Complementarity-determining regions (CDRs)
INPGSDRT

SEQ ID NO: 402 - protein, 10 aa, synthetic construct
  REGION 1..10: note = Antibody Complementarity-determining regions (CDRs)
ATDSSGNGDY

SEQ ID NO: 403 - protein, 5 aa, synthetic construct
  REGION 1..5: note = Antibody Complementarity-determining regions (CDRs)
SSVNY

SEQ ID NO: 404 - skipped

SEQ ID NO: 405 - protein, 10 aa, synthetic construct
  REGION 1..10: note = Antibody Complementarity-determining regions (CDRs)
QQWSSNPPLT

SEQ ID NO: 406 - protein, 8 aa, synthetic construct
  REGION 1..8: note = Antibody Complementarity-determining regions (CDRs)
GNAFSSYW

SEQ ID NO: 407 - protein, 8 aa, synthetic construct
  REGION 1..8: note = Antibody Complementarity-determining regions (CDRs)
IHPGDDDS

SEQ ID NO: 408 - protein, 9 aa, synthetic construct
  REGION 1..9: note = Antibody Complementarity-determining regions (CDRs)
ARGEVRLDY

SEQ ID NO: 409 - protein, 5 aa, synthetic construct
  REGION 1..5: note = Antibody Complementarity-determining regions (CDRs)
SSVNY

SEQ ID NO: 410 - skipped

SEQ ID NO: 411 - protein, 9 aa, synthetic construct
  REGION 1..9: note = Antibody Complementarity-determining regions (CDRs)
QQWSSYPLT

SEQ ID NO: 412 - protein, 8 aa, synthetic construct
  REGION 1..8: note = Antibody Complementarity-determining regions (CDRs)
GFIFSNYW

SEQ ID NO: 413 - protein, 10 aa, synthetic construct
  REGION 1..10: note = Antibody Complementarity-determining regions (CDRs)
IRVKSKNFAT

SEQ ID NO: 414 - protein, 14 aa, synthetic construct
  REGION 1..14: note = Antibody Complementarity-determining regions (CDRs)
TRTYYSNYNWYFDV

SEQ ID NO: 415 - protein, 5 aa, synthetic construct
  REGION 1..5: note = Antibody Complementarity-determining regions (CDRs)
SSVSY

SEQ ID NO: 416 - skipped

SEQ ID NO: 417 - protein, 9 aa, synthetic construct
  REGION 1..9: note = Antibody Complementarity-determining regions (CDRs)
QQYHSDPLT

SEQ ID NO: 418 - protein, 8 aa, synthetic construct
  REGION 1..8: note = Antibody Complementarity-determining regions (CDRs)
GFSFSSFG

SEQ ID NO: 419 - protein, 8 aa, synthetic construct
  REGION 1..8: note = Antibody Complementarity-determining regions (CDRs)
ISSGSRII

SEQ ID NO: 420 - protein, 14 aa, synthetic construct
  REGION 1..14: note = Antibody Complementarity-determining regions (CDRs)
ARSDRYDVYYSIDY

SEQ ID NO: 421 - protein, 11 aa, synthetic construct
  REGION 1..11: note = Antibody Complementarity-determining regions (CDRs)
QSLVHSNGNTY

SEQ ID NO: 422 - skipped

SEQ ID NO: 423 - protein, 10 aa, synthetic construct
  REGION 1..10: note = Antibody Complementarity-determining regions (CDRs)
SQSTHVPPLT

SEQ ID NO: 424 - protein, 8 aa, synthetic construct
  REGION 1..8: note = Antibody Complementarity-determining regions (CDRs)
GFTFSTFG

SEQ ID NO: 425 - protein, 8 aa, synthetic construct
  REGION 1..8: note = Antibody Complementarity-determining regions (CDRs)
ISSDSRII

SEQ ID NO: 426 - protein, 14 aa, synthetic construct
  REGION 1..14: note = Antibody Complementarity-determining regions (CDRs)
ARSDRYDVYYSMNY

SEQ ID NO: 427 - protein, 11 aa, synthetic construct
  REGION 1..11: note = Antibody Complementarity-determining regions (CDRs)
QSIVHSNGNTY

SEQ ID NO: 428 - skipped

SEQ ID NO: 429 - protein, 9 aa, synthetic construct
  REGION 1..9: note = Antibody Complementarity-determining regions (CDRs)
FQGSHVPYT

SEQ ID NO: 430 - protein, 8 aa, synthetic construct
  REGION 1..8: note = Antibody Complementarity-determining regions (CDRs)
GYTFTNYV

SEQ ID NO: 431 - protein, 8 aa, synthetic construct
  REGION 1..8: note = Antibody Complementarity-determining regions (CDRs)
INPDNDGA

SEQ ID NO: 432 - protein, 10 aa, synthetic construct
  REGION 1..10: note = Antibody Complementarity-determining regions (CDRs)
ARAARVWFPY

SEQ ID NO: 433 - protein, 5 aa, synthetic construct
  REGION 1..5: note = Antibody Complementarity-determining regions (CDRs)
SRVNY

SEQ ID NO: 434 - skipped

SEQ ID NO: 435 - protein, 9 aa, synthetic construct
  REGION 1..9: note = Antibody Complementarity-determining regions (CDRs)
QQWSTNPPT

SEQ ID NO: 436 - protein, 8 aa, synthetic construct
  REGION 1..8: note = Antibody Complementarity-determining regions (CDRs)
GFTFTDYY

SEQ ID NO: 437 - protein, 10 aa, synthetic construct
  REGION 1..10: note = Antibody Complementarity-determining regions (CDRs)
IRNKANGYTT

SEQ ID NO: 438 - protein, 14 aa, synthetic construct
  REGION 1..14: note = Antibody Complementarity-determining regions (CDRs)
ARDPLYGRSYALDY

SEQ ID NO: 439 - protein, 5 aa, synthetic construct
  REGION 1..5: note = Antibody Complementarity-determining regions (CDRs)
SRVNY

SEQ ID NO: 440 - skipped

SEQ ID NO: 441 - protein, 9 aa, synthetic construct
  REGION 1..9: note = Antibody Complementarity-determining regions (CDRs)
QQWSTNPPT

SEQ ID NO: 442 - protein, 8 aa, synthetic construct
  REGION 1..8: note = Antibody Complementarity-determining regions (CDRs)
GFTFSSYA

SEQ ID NO: 443 - protein, 8 aa, synthetic construct
  REGION 1..8: note = Antibody Complementarity-determining regions (CDRs)
ISNGGSYT

SEQ ID NO: 444 - protein, 10 aa, synthetic construct
  REGION 1..10: note = Antibody Complementarity-determining regions (CDRs)
ARDRNGYFDY

SEQ ID NO: 445 - protein, 7 aa, synthetic construct
  REGION 1..7: note = Antibody Complementarity-determining regions (CDRs)
SSVRSSY

SEQ ID NO: 446 - skipped

SEQ ID NO: 447 - protein, 11 aa, synthetic construct
  REGION 1..11: note = Antibody Complementarity-determining regions (CDRs)
HQYHRSPPTWT

SEQ ID NO: 448 - protein, 8 aa, synthetic construct
  REGION 1..8: note = Antibody Complementarity-determining regions (CDRs)
GFTFSSFG

SEQ ID NO: 449 - protein, 8 aa, synthetic construct
  REGION 1..8: note = Antibody Complementarity-determining regions (CDRs)
ISTGSSII

SEQ ID NO: 450 - protein, 14 aa, synthetic construct
  REGION 1..14: note = Antibody Complementarity-determining regions (CDRs)
ARSDRYDVYYDMDY

SEQ ID NO: 451 - protein, 12 aa, synthetic construct
  REGION 1..12: note = Antibody Complementarity-determining regions (CDRs)
QSLFNSGNQKNY

SEQ ID NO: 452 - skipped

SEQ ID NO: 453 - protein, 9 aa, synthetic construct
  REGION 1..9: note = Antibody Complementarity-determining regions (CDRs)
QNDYSYPWT

SEQ ID NO: 454 - protein, 8 aa, synthetic construct
  REGION 1..8: note = Antibody Complementarity-determining regions (CDRs)
GYSFTGYN

SEQ ID NO: 455 - protein, 8 aa, synthetic construct
  REGION 1..8: note = Antibody Complementarity-determining regions (CDRs)
IDPYYGGT

SEQ ID NO: 456 - protein, 14 aa, synthetic construct
  REGION 1..14: note = Antibody Complementarity-determining regions (CDRs)
ARYYRYDVGGYFDV

SEQ ID NO: 457 - protein, 5 aa, synthetic construct
  REGION 1..5: note = Antibody Complementarity-determining regions (CDRs)
SSVSY

SEQ ID NO: 458 - skipped

SEQ ID NO: 459 - protein, 9 aa, synthetic construct
  REGION 1..9: note = Antibody Complementarity-determining regions (CDRs)
QQWSSNPLT

SEQ ID NO: 460 - protein, 8 aa, synthetic construct
  REGION 1..8: note = Antibody Complementarity-determining regions (CDRs)
GFTFSSFG

SEQ ID NO: 461 - protein, 8 aa, synthetic construct
  REGION 1..8: note = Antibody Complementarity-determining regions (CDRs)
ISSGNTTI

SEQ ID NO: 462 - protein, 10 aa, synthetic construct
  REGION 1..10: note = Antibody Complementarity-determining regions (CDRs)
ARYFFYAMDY

SEQ ID NO: 463 - protein, 5 aa, synthetic construct
  REGION 1..5: note = Antibody Complementarity-determining regions (CDRs)
STVSY

SEQ ID NO: 464 - skipped

SEQ ID NO: 465 - protein, 9 aa, synthetic construct
  REGION 1..9: note = Antibody Complementarity-determining regions (CDRs)
QQRSSFPFT

SEQ ID NO: 466 - protein, 8 aa, synthetic construct
  REGION 1..8: note = Antibody Complementarity-determining regions (CDRs)
GFTFSTFG

SEQ ID NO: 467 - protein, 8 aa, synthetic construct
  REGION 1..8: note = Antibody Complementarity-determining regions (CDRs)
ISRGSIII

SEQ ID NO: 468 - protein, 14 aa, synthetic construct
  REGION 1..14: note = Antibody Complementarity-determining regions (CDRs)
VRSDRYDVYYDMDY

SEQ ID NO: 469 - protein, 4 aa, synthetic construct
  REGION 1..4: note = Antibody Complementarity-determining regions (CDRs)
SSVN

SEQ ID NO: 470 - skipped

SEQ ID NO: 471 - protein, 9 aa, synthetic construct
  REGION 1..9: note = Antibody Complementarity-determining regions (CDRs)
HQRSSYPPT

SEQ ID NO: 472 - protein, 8 aa, synthetic construct
  REGION 1..8: note = Antibody Complementarity-determining regions (CDRs)
GFTFSGFG

SEQ ID NO: 473 - protein, 8 aa, synthetic construct
  REGION 1..8: note = Antibody Complementarity-determining regions (CDRs)
ISTGSTII

SEQ ID NO: 474 - protein, 14 aa, synthetic construct
  REGION 1..14: note = Antibody Complementarity-determining regions (CDRs)
ARSDRYDVYYDMDY

SEQ ID NO: 475 - protein, 6 aa, synthetic construct
  REGION 1..6: note = Antibody Complementarity-determining regions (CDRs)
QDITNF

SEQ ID NO: 476 - skipped

SEQ ID NO: 477 - protein, 9 aa, synthetic construct
  REGION 1..9: note = Antibody Complementarity-determining regions (CDRs)
QQVNTLPPT

SEQ ID NO: 478 - protein, 8 aa, synthetic construct
  REGION 1..8: note = Antibody Complementarity-determining regions (CDRs)
GFTFTDAW

SEQ ID NO: 479 - protein, 10 aa, synthetic construct
  REGION 1..10: note = Antibody Complementarity-determining regions (CDRs)
VRSKAKNYAT

SEQ ID NO: 480 - protein, 10 aa, synthetic construct
  REGION 1..10: note = Antibody Complementarity-determining regions (CDRs)
TRSYVWYFDV

SEQ ID NO: 481 - protein, 5 aa, synthetic construct
  REGION 1..5: note = Antibody Complementarity-determining regions (CDRs)
SSVSY

SEQ ID NO: 482 - skipped

SEQ ID NO: 483 - protein, 9 aa, synthetic construct
  REGION 1..9: note = Antibody Complementarity-determining regions (CDRs)
QQRSSYPVT

SEQ ID NO: 484 - protein, 8 aa, synthetic construct
  REGION 1..8: note = Antibody Complementarity-determining regions (CDRs)
GYAFTNYL

SEQ ID NO: 485 - protein, 8 aa, synthetic construct
  REGION 1..8: note = Antibody Complementarity-determining regions (CDRs)
INPGSGGT

SEQ ID NO: 486 - protein, 15 aa, synthetic construct
  REGION 1..15: note = Antibody Complementarity-determining regions (CDRs)
ARWDFDAGGLNLIAY

SEQ ID NO: 487 - protein, 6 aa, synthetic construct
  REGION 1..6: note = Antibody Complementarity-determining regions (CDRs)
QDINNY

SEQ ID NO: 488 - skipped

SEQ ID NO: 489 - protein, 9 aa, synthetic construct
  REGION 1..9: note = Antibody Complementarity-determining regions (CDRs)
LQYEEFPLT

SEQ ID NO: 490 - protein, 8 aa, synthetic construct
  REGION 1..8: note = Antibody Complementarity-determining regions (CDRs)
GYTFTNYW

SEQ ID NO: 491 - protein, 8 aa, synthetic construct
  REGION 1..8: note = Antibody Complementarity-determining regions (CDRs)
IDPSDSET

SEQ ID NO: 492 - protein, 8 aa, synthetic construct
  REGION 1..8: note = Antibody Complementarity-determining regions (CDRs)
ASYDWFAY

SEQ ID NO: 493 - protein, 7 aa, synthetic construct
  REGION 1..7: note = Antibody Complementarity-determining regions (CDRs)
SSVRSSY

SEQ ID NO: 494 - skipped

SEQ ID NO: 495 - protein, 11 aa, synthetic construct
  REGION 1..11: note = Antibody Complementarity-determining regions (CDRs)
HQYHRSPPTWT

SEQ ID NO: 496 - protein, 608 aa, synthetic construct
  source 1..608: note = Heavy chain sequence of <signal peptide><Ab19-heavy variable region><IgG1 constant region with N297A mutation, without stop codon><short linker><IL2-cytokine sequence>stop codon
MGWSCIILFLVATATGVHSQVQLQQPGAEPLKPGASVKMSCEASGYTFTNFWITWMKLRPGQGLEWIGDIYPGTGNTNYNEKFKSRATLTVDTSSTTAYMQLSSLTSEDSAVYYCAREAYYTNPGFAYWGHGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 497 - protein, 617 aa, synthetic construct
  source 1..617: note = Heavy chain sequence of <signal peptide><Ab19-heavy variable region><IgG1 constant region with N297A mutation, without stop codon><long linker><IL2-cytokine sequence>stop codon
MGWSCIILFLVATATGVHSQVQLQQPGAEPLKPGASVKMSCEASGYTFTNFWITWMKLRPGQGLEWIGDIYPGTGNTNYNEKFKSRATLTVDTSSTTAYMQLSSLTSEDSAVYYCAREAYYTNPGFAYWGHGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 498 - protein, 238 aa, synthetic construct
  source 1..238: note = Light chain sequence of <signal peptide>< Ab19-light variable region><kappa constant region with stop codon>
MGWSCIILFLVATATGVHSDVLMTQSPLSLPVNLGDQASISCKSSQNIVHSNGNTYLEWYLQRPGQSPKLLIYKVSNRFSGVPDRFSGSGSGTDFTLKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 499 - protein, 238 aa, synthetic construct; identical to SEQ ID NO: 498
  source 1..238: note = Light chain sequence of <signal peptide>< Ab19-light variable region><kappa constant region with stop codon>

SEQ ID NO: 500 - DNA, 7742 bp, synthetic construct
  source 1..7742: mol_type = other DNA; plasmid = TREM2-CAR-BF synthetic plasmid
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagccctcgagaagcttgccaccatgggtgtccctacccagctcctgggactgctcctgctgtggatcaccgacgccatctgcgatgttttgatgacccaaagtccgctctccctgcctgtcaatcttggagatcaagcctccatctcttgcaaatctagccagaacattgtacatagtaatggaaacacctatttagaatggtacctgcagagaccaggccagtctccaaagctccttatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaaggcggaggcggaagtggaggcggaggatctggcggcggaggctctcaggtccaactgcagcagcctggggctgagccactgaagcctggggcttcagtgaagatgtcctgcgaggcttctggctacaccttcaccaacttctggataacctggatgaagctgaggcctggacaaggccttgagtggattggagatatttatcctggtactggtaataccaactataatgagaagttcaagagcagggccacactgactgtggacacatcctccaccacagcctacatgcagctcagcagcctgacatctgaggactcagcggtctattactgtgcaagagaggcctactatactaaccccgggtttgcttactggggccacgggactctggtcactgtctctgcaatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagaggcggcgggtccggaggagagggcagaggaagtctcctaacatgcggtgacgtggaggagaatcctggcccaatgggcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccaggggatccccgacttcttcaagcagtccttccccgagggcttcacatgggagagggtcaccacctacgaggacgggggcgtgctgaccgccacccaggacaccagcctccaggacgggtgcctcatctacaacgtcaagatcaggggcgtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgagtcgacggtaccgcgggcccgggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 501 - DNA, 22 bp, synthetic construct
  source 1..22: mol_type = other DNA; note = CCL23 forward primer
ccgtgttcactcctggagagtt

SEQ ID NO: 502 - DNA, 22 bp, synthetic construct
  source 1..22: mol_type = other DNA; note = S100A8 forward primer
atgccgtctacagggatgacct

SEQ ID NO: 503 - DNA, 21 bp, synthetic construct
  source 1..21: mol_type = other DNA; note = S100A9 forward primer
gcacccagacaccctgaacca

SEQ ID NO: 504 - DNA, 20 bp, synthetic construct
  source 1..20: mol_type = other DNA; note = IFI6 forward primer
gctagagtgcagtggctatt

SEQ ID NO: 505 - DNA, 24 bp, synthetic construct
  source 1..24: mol_type = other DNA; note = Ccl18 forward primer
gttgactattctgaaaccagcccc

SEQ ID NO: 506 - DNA, 23 bp, synthetic construct
  source 1..23: mol_type = other DNA; note = CCL23 Reverse primer
gcttcagcattctcacgcaaacc

SEQ ID NO: 507 - DNA, 24 bp, synthetic construct
  source 1..24: mol_type = other DNA; note = S100A8 Reverse primer
agaatgaggaactcctggaagtta

SEQ ID NO: 508 - DNA, 22 bp, synthetic construct
  source 1..22: mol_type = other DNA; note = S100A9 Reverse primer
tgtgtccaggtcctccatgatg

SEQ ID NO: 509 - DNA, 23 bp, synthetic construct
  source 1..23: mol_type = other DNA; note = IFI6 Revers primer
gtaatcctacttgggaggttgag

SEQ ID NO: 510 - DNA, 23 bp, synthetic construct
  source 1..23: mol_type = other DNA; note = Ccl18 Reverse primer
gtcgctgatgtatttctggaccc